(19) United States
(12) Patent Application Publication
(10) Pub. No.: US 2010/0035865 A1
(43) Pub. Date: Feb. 11, 2010

(54) Sulfonamides and Pharmaceutical Compositions Thereof

(75) Inventors: Kimberly Gail Estep, Groton, CT (US); Anton F.J. Fliri, Stonington, CT (US); Christopher J. O'Donnell, Mystic, CT (US)

Correspondence Address: PFIZER INC;Mary J Hosley, 150 EAST 42ND STREET, MS: 150/02/E112, NEW YORK, NY, 10017-5612, US

(73) Assignee: PFIZER INC

(21) Appl. No.: 12/527,868

(22) PCT Filed: Mar. 26, 2008
(86) PCT No.: PCT/IB08/00784

§ 371 Date: Aug. 20, 2009

Related U.S. Application Data
(60) Provisional application No. 60/921,615, filed on Apr. 3, 2007.

(51) Int. Cl. A61K 31/451; C07D 211/40; C07D 239/12; C07D 265/10; C07D 319/06; C07D 309/30; C07D 285/16; C07D 279/02; A61K 31/505; A61K 31/535; A61K 31/365; A61K 31/357; A61K 31/549; A61K 31/541; A61P 25/00; A61P 9/00; A61P 25/28; A61P 25/16; A61P 27/02; A61P 21/00; A61P 25/06; A61P 25/22

(52) U.S. Cl. 514/222.5; 546/216; 544/317; 544/97; 549/228; 549/293; 544/8; 544/3; 514/327; 514/274; 514/228.8; 514/452; 514/459; 514/222.2

(57) ABSTRACT

The invention is directed to a class of compounds, including the pharmaceutically acceptable salts of the compounds, having the structure of formula (I), as defined in the specification. The invention is also directed to compositions containing the compounds of formula (I).

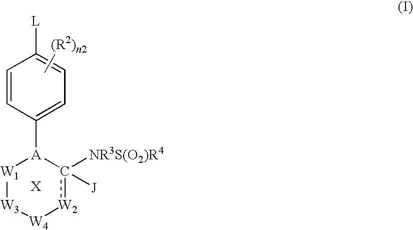

### FIELD OF THE INVENTION

The present invention comprises a novel class of compounds having the structure of formula I as defined herein and pharmaceutical compositions comprising a compound of formula I. The present invention also comprises methods of treating a subject by administering a therapeutically effective amount of a compound of formula I to the subject. These compounds are useful for the conditions disclosed herein. The present invention further comprises methods for making the compounds of formula I and corresponding intermediates.

### BACKGROUND OF THE INVENTION

The present invention provides compounds of formula I, pharmaceutical compositions thereof, and methods of using the same, processes for preparing the same, and intermediates thereof.

The primary excitatory neurotransmitter in the mammalian central nervous system (CNS) is the amino acid glutamate whose signal transduction is mediated by either ionotropic or metabotropic glutamate receptors (GluR). Ionotropic glutamate receptors (iGluR) are comprised of three subtypes differentiated by their unique responses to the three selective iGluR agonists □-amino-3-hydroxy-5-methylisoxazole-4-propionic acid (AMPA), N-methyl-D-aspartate (NMDA) and kainate (Parsons C G, Danysz W and Lodge D (2002), in: Ionotropic Glutamate Receptors as Therapeutic Targets (Danysz W. Lodge D and Parsons C G eds), pp 1-30, F. P. Graham Publishing Co., Tennessee). AMPA receptors, proteinaceous homo- or heterotetramers comprised of any combination of four ca. 900 amino acid monomer subunits each encoded from a distinct gene (Glu_{A1-A4}) with each subunit protein existing as one of two splice variants deemed “flip” and “flop”, mediate the vast majority of excitatory synaptic transmissions in the mammalian brain and have long been proposed to be an integral component of the neural circuitry that mediates cognitive processes (Bleakman D and Lodge D (1998) Neuropharmacology of AMPA and Kainate Receptors. Neuropharmacology 37:1187-1204). The combination of various heterotetrameric possibilities, two splice forms for each of the four iGluR monomers and receptor subunit RNA editing with the heterogeneous distribution of AMPA receptors throughout the brain highlight the myriad of potential AMPA receptor responses within this organ (Black M D (2005) Therapeutic Potential of Positive AMPA Modulators and Their Relationship to AMPA Receptor Subunits. A Review of Preclinical Data. Psychopharmacology 179:154-163).

AMPA receptors are ion channels that mediate the cellular influx of Na⁺ and Ca²⁺ resulting in neuronal membrane depolarization. AMPA receptors may also stimulate NMDA receptors indirectly since its induced membrane depolarization can remove the Mg²⁺ blockade of NMDA receptors leading to their activation. The AMPA-mediated change in electrophysiological current occurs upon activation of the receptor by its endogenous agonist glutamate. Such change in voltage is ephemeral, with its amplitude and duration dependent upon ion channel opening mediated by either the interval of agonist site occupation by glutamate (known as deactivation) or the temporal molecular disruption of the open ion channel with glutamate binding intact (known as desensitization). In either case, AMPA receptor-mediated ion influx may be prolonged by a compound which slows either deactivation via glutamate dissociation from the AMPA receptor agonist site or desensitization of the glutamate-bound AMPA receptor (Lynch G and Gall C M (2006) Ampakines and the Threefold Path to Cognitive Enhancement. TRENDS in Neuroscience 29:554-562). Such compounds which slow the rate of AMPA receptor deactivation and/or desensitization in the presence of glutamate are coined AMPA positive allosteric modulators (PAMs) or AMPA receptor potentiators.

Numerous in vitro and in vivo studies have demonstrated the ability of AMPA receptor potentiators to enhance excitatory post-synaptic pulses, the requisites for long-term potentiation and increased neuronal plasticity that are believed to manifest in nootropic effects (Staubli U, Rogers G and Lynch G (1994) Facilitation of Glutamate Receptors Enhances Memory. Proceedings of the National Academy of Sciences of the United States of America 91:777-781; O'Neill M J, Bleakman D, Zimmerman D M and Nisenbaum E S (2004) AMPA Receptor Potentiators for the Treatment of CNS Disorders. Current Drug Targets—CNS & Neurological Disorders 3:181-194; Lynch and Gall, 2006). Furthermore, several reports suggest that various AMPA potentiators have beneficial cognitive effects in rodents (Staubli et al., 1994; Larson J, Lieu T, Petchpradub V, LeDuc B, Ngo H, Rogers G A and Lynch G (1995) Facilitation of Olfactory Learning by a Modulator of AMPA Receptors. The Journal of Neuroscience 15:8023-8030; Rogan M T, Staubli U V and LeDoux J E (1997) AMPA Receptor Facilitation Accelerates Fear Learning without Altering the Level of Conditioned Fear Acquired. The Journal of Neuroscience 1997:5928-5935; Hampson R E, Rogers G A, Lynch G and Deadwyler S A (1998) Facilitative Effects of the Ampakine CX516 on Short-Term Memory in Rats: Enhancement of Delayed-Nonmatch-to-Sample Performance. The Journal of Neuroscience 18:2740-2747; Lebrun C, Pilliere E and Lestage P (2000) Effects of S 18986-1, a Novel Cognitive Enhancer, on Memory Performances in an Object Recognition Task in Rats, European Journal of Pharmacology 401:205-212; Zushida K, Sakurai M, Wada K and Sekiguchi M (2007) Facilitation of Extinction Learning for Contextual Fear Memory by PEPA: A Potentiator of AMPA Receptors. The Journal of Neuroscience 27:158-166), monkeys (Buccafusco J J, Weiser T. Winter K, Klinder K and Terry Jr. A V (2004) The Effects of IDRA 21, a Positive Modulator of the AMPA Receptor, on Delayed Matching Performance by Young and Aged Rhesus Monkeys. Neuropharmacology 46:10-22; Porrino L J, Daunais J B R, Gary A., Hampson R E and Deadwyler S A (2005) Facilitation of Task Performance and Removal of the Effects of Sleep Deprivation by an Ampakine (CX717) in Nonhuman Primates. PLoS Biology 3:1639-1652) and humans (Lynch G, Kessler M, Rogers G, Ambros-Ingerson J, Granger R and Schehr R S (1996) Psychological Effects of a Drug that Facilitates Brain AMPA Receptors. International Clinical Psychopharmacology 11:13-19; Ingvar M, Ambros-Ingerson J, Davis M, Granger R, Kessler M, Rogers G A, Schehr R S and Lynch G (1997) Enhancement by an Ampakine of Memory Encoding in Humans. Experimental Neurology 146:553-559; Lynch G, Granger R, Ambros-Ingerson J, Davis C M, Kessler M and Schehr R S (1997) Evidence That a Positive Modulator of AMPA-Type Glutamate Receptors Improves Delayed Recall in Aged Humans. Experimental Neurology 145;89-92; Goff D C, Leahy L, Berman I, Posever T, Herz L, Leon A C, Johnson S A and Lynch G (2001) A Placebo-Controlled Pilot Study of the Ampakine CX516 Added to Clozapine in Schizophrenia. Journal of Clinical Psychopharmacology 21:484-487). Hypofunction of glutamatergic neurotransmission has been implicated in the pathophysiology underlying schizophrenia. Additionally, postmortem studies have identified alterations in glutamate receptor density and subunit composition in brain regions that exhibit impaired task-evoked activation in schizophrenia including the prefrontal cortex, thalamus and temporal lobe (Gao X-M, Sakai K, Roberts R C, Conley R R, Dean B and Tamminga C A (2000) Ionotropic Glutamate Receptors and Expression of N-Methyl-D-Aspartate Receptor Subunits in Subregions of Human Hippocampus: Effects of Schizophrenia. American Journal of Psychiatry 157:1141-1149; Ibrahim H M, Hogg A J, Healy D J, Haroutunian V, Davis K L and Meador-Woodruff J H (2000) Ionotropic Glutamate Receptor Binding and Subunit mRNA Expression in Thalamic Nuclei Schizophrenia. American Journal of Psychiatry 157:1811-1823; Meador-Woodruff J H and Healy D J (2000) Glutamate Receptor Expression in Schizophrenic Brain. Brain Research Reviews 31:288-294). With this in mind, a clear unmet medical need for various neurological disorders exists for AMPA receptor potentiators. Such neuropsychiatric conditions potentially treatable with AMPA receptor potentiators include, for example:

acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

A need still exists for new drug therapies for the treatment of subjects suffering from or susceptible to the above disorders or conditions. In particular, a need still exists for new drugs having one or more improved properties (such as safety profile, efficacy, or physical properties) relative to those currently available.

### SUMMARY OF THE INVENTION

The invention is directed to a class of compounds, including the pharmaceutically acceptable salts of the compounds, having the structure of formula I:

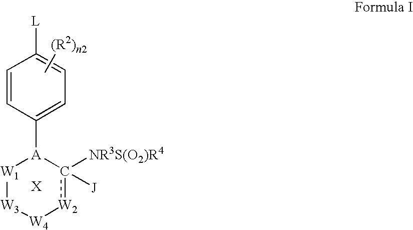

wherein

-L is

a) —Br, —I, —Cl, —O—S(O₂)-alkyl, wherein the —O—S(O₂)-alkyl is optionally substituted with halogen,

b)

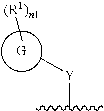

or

c)

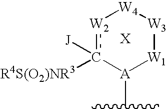

wherein ring G is aryl, heteroaryl, cycloalkyl, or heterocycloalkyl;

each of groups W₁, W₃, and W₄ in each ring X is independently selected from the group consisting of —(CHR¹²)ₐ—, —S(O)₂—, —C(O)—, —O—, —S—, and —NR⁵—;

group W₂ in each ring X is selected from the group consisting of —(CHR¹²)ₐ—, —S(O)₂—, —C(O)—, —O—, —S—, —NR⁵— and N;

J is hydrogen or is absent;

the bond [symbol] between W₂ and C is a single or double bond;

a is independently at each occurrence 1 or 2, provided that if W₃ or W₄ is —(CHR¹²)ₐ—, a is 1;

with the proviso that
(a) a ring X does not contain more than one group selected from —S(O)₂— and —C(O)—;
(b) a ring X contains between one and two ring heteroatoms selected from nitrogen, sulfur or oxygen, wherein if a ring X contains two heteroatoms, then either (i) the two ring heteroatoms are each bonded to a —C(O)— group, or (ii) the two ring heteroatoms are a nitrogen of an —NR⁵— group and a sulfur of an —S(O₂)— group, and the nitrogen and sulfur are directly bonded to each other;
(c) if W₂═N, J is absent and [symbol] is a double bond; and
(d) if both rings X are present, then W₁, W₂, W₃ and W₄ of one ring X are the same as, respectively, W₁, W₂, W₃ and W₄ of the other ring X;
Y is absent or is —O—, —(CR²¹R²²—)ₙ₃, —CR²¹R²²O—, —NR²¹C(O)—, —NR²¹S(O₂), —NR²¹C(O)NR²²—, S(O), or S(O₂);

n3 is 1 or 2;

R²¹ and R²² are each independently hydrogen, alkyl or aryl;

A is C—B, where B is hydrogen, alkyl, halogen, hydroxyl, alkoxy, amino, alkylamino, or dialkylamino;

with the proviso that if W₁ is —O— or —NR⁵—, B is hydrogen, alkyl, hydroxyl or alkoxy;

R³ is hydrogen, alkyl, cycloalkyl, or heterocycloalkyl, wherein each R³ alkyl, cycloalkyl, or heterocycloalkyl is optionally substituted with halogen, —CN, alkoxy, hydroxyl, alkyl, cycloalkyl, heterocycloalkyl, aryl or heteroaryl;

R⁴ is alkyl, alkenyl, cycloalkyl, cycloalkenyl, heterocycloalkyl, aryl, heteroaryl, or NR⁵⁵R⁶⁶, wherein each R⁴ is optionally substituted with halogen, —CN, alkoxy, hydroxyl, alkyl, cycloalkyl, heterocycloalkyl, aryl, heteroaryl;

each of R⁵⁵ and R⁶⁶ is independently hydrogen, alkyl or cycloalkyl, wherein the alkyl or cycloalkyl R⁵⁵ or R⁶⁶ is optionally independently substituted with —R¹⁰¹, —OR¹⁰¹, —C(O)R¹⁰³, or S(O₂)R¹⁰³;

or R⁵⁵ and R⁶⁶ together with the nitrogen they are attached to form a heterocyclic ring which is optionally substituted with one or more alkyl, halogen, or —OR¹⁰¹,

each R⁵ is independently at each occurrence hydrogen alkyl, —C(O)R⁷, —C(O)OR⁷, —C(O)NR⁷R⁸, —S(O₂)R⁷, cycloalkyl, heterocycloalkyl, aryl, or heteroaryl, wherein each R⁵ alkyl, cycloalkyl, heterocycloalkyl, aryl, or heteroaryl is optionally substituted with halogen, —CN, alkoxy, hydroxyl, alkyl, cycloalkyl, heterocycloalkyl, aryl or heteroaryl,

with the proviso that when R⁵ is —C(O)OR⁷ or —C(O)NR⁷R⁸, at least one group bound to the nitrogen of NR⁵ is (CHR¹²);

each of R⁸ and R⁷ is alkyl, cycloalkyl, heterocycloalkyl, wherein each of R⁸ and R⁷ is optionally substituted with halogen, —CN, alkoxy, hydroxyl, alkyl, cycloalkyl, heterocycloalkyl, aryl or heteroaryl;

n1 and n2 are each independently 1, 2, 3 or 4;

each of R¹, R² and R¹² is independently at each occurrence hydrogen, halogen, hydroxyl, alkoxy, cyano, nitro, amino, alkylamino, dialkylamino, C(O)NH₂, C(O)NH(alkyl), C(O)N(alkyl)₂, OC(O)alkyl, C(O)Oalkyl, alkyl, aryl, heteroaryl, heterocycloalkyl, cycloalkyl, or alkyl-S(O)₂—NH—, wherein the R¹, R² and R¹² alkoxy, alkylamino, dialkylamino, C(O)NH(alkyl), C(O)N(alkyl)₂, C(O)Oalkyl, alkyl, aryl, heteroaryl, heterocycloalkyl, cycloalkyl or alkyl-S(O)₂—NH— are each independently optionally substituted with one, two, three or four R⁴¹, wherein each R⁴¹ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —OC(O)OR¹⁰¹, —C(O)NR¹⁰¹R¹⁰², —S(O₂)NR¹⁰¹R¹⁰², —NR¹⁰¹R¹⁰², NR¹⁰¹(O)R¹⁰³, and —NR¹⁰¹S(O)₂R¹⁰³ wherein each of the R⁴¹ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹(S)₂R¹⁰³, —OC(O)R¹⁰³, —C(O)OR¹⁰³, —C(O)NR¹⁰¹R¹⁰², —NR¹⁰¹C(O)R¹⁰³, —NR¹⁰¹C(O)N(R¹⁰³)₂, and —C(O)R¹⁰³;

q is 0, 1 or 2;

or when R¹ is aryl or heteroaryl, two R⁴¹ substituents bonded to adjacent carbon atoms of R¹, together with the adjacent carbon atoms, form a heterocyclic or carbocycli ring which is optionally substituted with one or more R¹⁰, wherein each R¹⁰ is independently selected from the group consisting of hydrogen, —CN, halogen, —C(O)R¹⁰¹, —C(O)NR¹⁰¹R¹⁰², NR¹⁰¹R¹⁰², —OR¹⁰¹ or —R¹⁰¹;

or, two R¹ substituents bonded to adjacent carbon atoms of ring G, together with the adjacent carbon atoms, form a heterocyclic or carbocyclic ring which is optionally substituted with one or more R¹⁰,

or, two R² substituents bonded to adjacent carbon atoms of the phenyl ring substituted by R², together with the adjacent carbon atoms, form a heterocyclic or carbocyclic ring which is optionally substituted with one or more R¹⁰;

each R¹⁰¹ and each R¹⁰² is independently selected from the group consisting of hydrogen, alkyl, alkenyl, alkynyl, cycloalkyl, aryl, heterocycloalkyl and heteroaryl;

wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;

each R¹⁰³ is independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl.

In Formula I, it is understood that when Y is —(CR²¹R²²—)ₙ₃, —CR²¹R²²O—, —NR²¹C(O)—, —NR²¹S(O₂), or —NR²¹C(O)NR²²—, either end-group of Y can be bound to ring G while the other end-group of Y can be bound to the phenyl group substituted by R². For example, if Y is —NR²¹C(O)—, either the N of the —NR²¹C(O)— group is bound to ring G and the C of the —NR²¹C(O)— group is bound to the phenyl group substituted by R², or the C of the —NR²¹C(O)— group is bound to ring G and the N of the —NR²¹C(O)— group is bound to the phenyl group substituted by R².

In one embodiment of the invention, Y is absent or is —NHC(O)—.

In another embodiment of the invention, ring G is phenyl substituted with R¹ which is optionally substituted as in formula I.

In another embodiment of the invention, the compound of formula I has the formula I′:

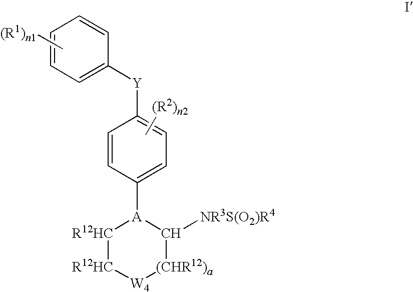

wherein W₄ is —NR⁵— or —O— and a is 1 or 2.

In another embodiment of the invention, the compound of formula I has the formula I″:

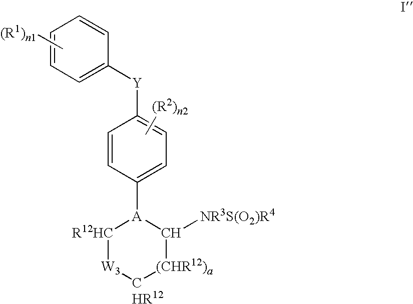

wherein W₃ is —NR⁵— or —O— and a is 1 or 2.

In another embodiment of the compound of formula I, I′ or I″, J is hydrogen and the groups

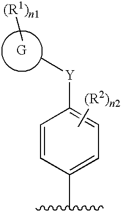

and —NR³S(O₂)R⁴ are in a trans relationship.

In an example of this embodiment of formula I′, —Y— is a direct bond, R³ is alkyl or hydrogen, R⁴ is alkyl, W₄ is NR⁵ where R⁵ is alkyl, a is 1, and the absolute stereochemistry is

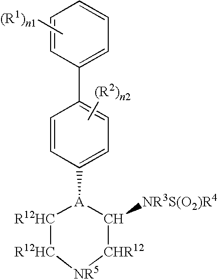

In another example of this embodiment of formula I′, —Y— is a direct bond, R³ is alkyl or hydrogen, R⁴ is alkyl, W₄ is NR⁵ where R⁵ is alkyl, a is 1, and the absolute stereochemistry is

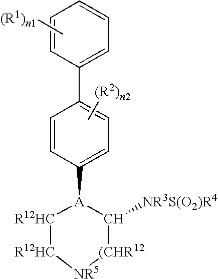

In another embodiment of the compound of formula I, I′ or I″, J is hydrogen and the groups

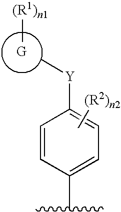

and —NR³S(O₂)R⁴ are in a cis relationship.

In another embodiment of the invention, ring G is heterocycloalkyl optionally substituted as in formula I.

In another embodiment of the invention, the compound of formula I has the formula I′″

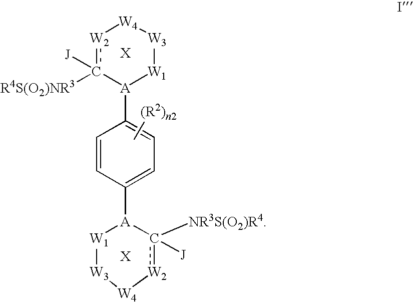

In another embodiment of the invention, B is hydroxyl or alkoxy. In one example of this embodiment, W₄ is —O—. In another example of this embodiment, W₃ is —O—.

In another embodiment of the invention, when Y is —NHC(O)—, W₄ is —NR⁵—.

In another embodiment of the invention, W₄═—NR⁵—.

In another embodiment of the invention, W₄═—O—.

In another embodiment of the invention, Y is —NR²¹C(O)—. As an example of this embodiment, Y is —NR²¹C(O)— wherein the nitrogen of the —NR²¹C(O)— group is bound to the ring substituted by (R²)ₙ₂.

In another embodiment of the invention, R⁵ is hydrogen, alkyl, cycloalkyl, heterocycloalkyl, —C(O )R⁷ or —S(O)₂R⁷.

In another embodiment of the invention, R⁴ is cycloalkyl, heterocycloalkyl, or alkyl, preferably methyl, ethyl, n-propyl, isopropyl, n-butyl, sec-butyl, isobutyl or t-butyl, wherein the R⁴ alkyl is optionally substituted with halogen.

In another embodiment of the invention, W₄ is —NR⁵— or —O— and W₃ is —C(O)—.

In another embodiment of the invention, W₁ is —NR⁵— or —O— and W₃ is —C(O)—.

In another embodiment of the invention, W₂ is —NR₅— and W₄ is —C(O)—.

In another embodiment of the invention, W₂ is —C(O)— and W₄ is —NR₅— or —O—.

In another embodiment of the invention, W₁ or W₃ is —S(O₂)—.

In another embodiment of the invention, ring G is phenyl having one or two R¹ substituents, where each R¹ is independently a heteroaryl which is preferably thiophenyl, cyano, halogen, alkyl, cycloalkyl, alkyl-NH—C(O)—, alkyl-S(O)₂—NH—, halophenyl, or dihalophenyl.

In another embodiment of the invention, each R¹ is independently hydrogen, phenyl, thiophenyl, halogen, alkoxy, hydroxyl, cyano, C(O)NH₂, C(O)NH(alkyl), C(O)N(alkyl)₂, OC(O)alkyl, C(O)Oalkyl, alkyl, heterocycloalkyl, or cycloalkyl and is independently optionally substituted with one, two, three or four R⁴¹, wherein each R⁴¹ is independently selected from the group consisting of halogen, —C(O)OR¹⁰¹, —OC(O)OR¹⁰¹, —S(O₂)NR¹⁰¹R¹⁰², and —NR¹⁰¹S(O)₂R¹⁰³. In an exemplary embodiment, each R¹ alkyl, or cycloalkyl is independently optionally substituted with one, two, three or four R⁴¹, wherein each R⁴¹ is independently selected from the group consisting of halogen, —C(O)OR¹⁰¹, —OC(O)OR¹⁰¹, —S(O₂)NR¹⁰¹R¹⁰², and —NR¹⁰¹S(O)₂R¹⁰³.

In another embodiment of the invention, ring G is phenyl and R¹ may be, for example, in the para position relative to Y. R¹ may be, as another example, in the ortho position relative to Y. For an example of this embodiment, R¹ is cyano or halogen, preferably chlorine, and is in the ortho or para position relative to Y.

In another embodiment of the invention, R¹ may also be, as another example, thiophenyl, which is preferably 3-thiophenyl, or dihalophenyl, which is preferably 2,4-dihalophenyl, more preferably 2,4-difluorophenyl.

In another embodiment of the invention, each R¹⁰¹ and each R¹⁰² is independently selected from the group consisting of hydrogen, alkyl, alkenyl, alkynyl, cycloalkyl, aryl, heterocycloalkyl and heteroaryl, wherein each R¹⁰¹ and R¹⁰² alkyl, alkenyl, alkynyl, cycloalkyl, aryl, heterocycloalkyl or heteroaryl is unsubstituted, and each R¹⁰³ is independently selected from the group consisting of alkyl, alkenyl, cycloalkyl, aryl, heterocycloalkyl and heteroaryl, wherein each R¹⁰³ is unsubstituted.

Exemplary compounds according to the invention include the specific compounds disclosed herein herein or pharmaceutically acceptable salts thereof.

The compounds of formula I are useful for the treatment or prevention of a variety of neurological and psychiatric disorders associated with glutamate dysfunction, including; acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, attention deficit disorder, and conduct disorder. Accordingly, in one embodiment, the invention provides a method for treating or preventing a condition in a mammal, such as a human, selected from the conditions above, comprising administering a compound of formula I to the mammal. The mammal is preferably a mammal in need of such treatment or prevention.

As an example, the invention provides a method for treating or preventing a condition selected from migraine, anxiety disorders, schizophrenia, and epilepsy. Exemplary anxiety disorders are generalized anxiety disorder, social anxiety disorder, panic disorder, post-traumatic stress disorder and obsessive-compulsive disorder. As another example, the invention provides a method for treating or preventing depression selected from Major Depression, Chronic Depression (Dysthymia), Seasonal Depression (Seasonal Affective Disorder), Psychotic Depression, and Postpartum Depression. As another example, the invention provides a method for treating or preventing a sleep disorder selected from insomnia and sleep deprivation.

In another embodiment, the invention comprises methods of treating or preventing a condition in a mammal, such as a human, by administering a compound having the structure of formula I, wherein the condition is selected from the group consisting of atherosclerotic cardiovascular diseases, cerebrovascular diseases and peripheral arterial diseases, to the mammal. The mammal is preferably a mammal in need of such treatment or prevention. Other conditions that can be treated or prevented in accordance with the present invention include hypertension and angiogenesis.

In another embodiment the present invention provides methods of treating or preventing neurological and psychiatric disorders associated with glutamate dysfunction, comprising: administering to a mammal, preferably a mammal in need thereof, an amount of a compound of formula I effective in treating or preventing such disorders. The compound of formula I is optionally used in combination with another active agent. Such an active agent may be, for example, an atypical antipsychotic or an AMPA potentiator. Accordingly, another embodiment of the invention provides methods of treating or preventing neurological and psychiatric disorders associated with glutamate dysfunction, comprising administering to a mammal an amount of a compound of formula I and further comprising administering an atypical antipsychotic or an AMPA potentiator.

The invention is also directed to a pharmaceutical composition comprising a compound of formula I, and a pharmaceutically acceptable carrier. The composition may be, for example, a composition for treating or preventing a condition selected from the group consisting of acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, social anxiety disorder, panic disorder, post-traumatic stress disorder and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder, wherein the composition contains an amount of the compound of formula I that is effective in the treatment or prevention of such conditions.

The composition may also further comprise another active agent. Such an active agent may be, for example, an atypical antipsychotic. Such an active agent may be, as another example, an AMPA potentiator.

### DETAILED DESCRIPTION OF THE INVENTION

This detailed description of embodiments is intended only to acquaint others skilled in the art with Applicants' invention, its principles, and its practical application so that others skilled in the art may adapt and apply the invention in its numerous forms as it may be best suited to the requirements of a particular use. This invention, therefore, is not limited to the embodiments described in this specification, and may be variously modified.

### Abbreviations and Definitions

TABLE A
Abbreviations

| | |
|---|---|
| 1-HOAT | 1-hydroxy-7-azabenzotriazole |
| 1-HOBt | 1-hydroxybenzotriazole hydrate |
| ADP | Adenosine diphosphate (the natural ligand of P2Y12) |
| AMP | Adenosine monophospate |
| ASA | Acetylsalicylic acid |
| ATP | Adenosine triphosphate |
| Bn | Benzyl group |
| Boc | tert-butoxycarbonyl |
| BOP-Cl | bis(2-oxo-3-oxazolidinyl)phosphinic chloride |
| br | Broad |
| BSA | Bovine serum albumin |
| Cbz | benzyloxycarbonyl |
| CD₃OD | Deuterated methanol |
| CDCl₃ | Deuterated chloroform |
| CDI | 1,1′-carbonyldiimidazole |
| d | Doublet |
| DBN | 1,5-diazabicyclo[4.3.0]non-5-ene |
| DBU | 1,8-diazabicyclo[5.4.0]undec-7-ene |
| DCC | 1,3-dicyclohexylcarbodiimide |
| DCM | dichloromethane |
| DMC | 2-chloro-1,3-dimethylimidazolinium chloride |
| dd | Doublet of doublets |
| DEPC | diethyl cyanophosphonate |
| DIEA | diisopropylethylamine |
| DMF | N,N-dimethylformamide |
| DMSO | dimethyl sulphoxide |
| DPBS | Dulbecco's Phosphate Buffered Saline |
| DPPA | Diphenylphosphoryl Azide |
| EBSS | Earle's Balanced Salt Solution |
| EDC | 1-(3-dimethylaminopropyl)-3-ethylcarbodiimide |
| | hydrochloride |
| EDTA | ethylenediaminetetraacetic acid |
| EGTA | ethyleneglycol-bis(β-aminoethyl)-N,N,N′,N′-tetraacetic Acid |
| ELSD | Evaporative Light Scattering Detection |
| ESI | Electrospray Ionization for mass spectrometry |
| Et₃N | triethylamine |
| EtOAc | ethyl acetate |
| EtOH | ethanol |
| FBS | Fetal bovine serum |
| Fmoc | Fluorene methyloxycarbonyl |
| HATU | O-(7-azabenzotriazol-1-yl)-N,N,N′,N′-tetramethyluronium |
| | hexafluorophosphate |
| HBTU | O-benzotriazol-1-yl-N,N,N′,N′-tetramethyluronium |
| | hexafluorophosphate |
| HCl | Hydrochloric acid |
| HEK | Human embryonic kidney |
| HEPES | 4-(2-hydroxyethyl)-1-Piperazineethane sulfonic acid |
| HOBT | 1-hydroxybenzotriazole |
| HRMS | High Resolution Mass Spectroscopy (electrospray ionization |
| | positive scan) |
| K₃PO₄ | Potassium phosphate |
| LCMS | Liquid Chromatography - Mass Spectroscopy |
| LRMS | Low Resolution Mass Spectroscopy (electrospray or |
| | thermospray ionization positive scan) |
| LRMS | Low Resolution Mass Spectroscopy (electrospray ionization |
| (ES⁻) | negative scan) |
| m | Multiplet |
| m/z | Mass spectrum peak |
| MEM | Minimum essential medium |
| MeOH | methanol |
| MHz | Megahertz |
| MS | Mass spectroscopy |
| MTBE | Methyl t-Butyl Ether |
| NaH | Sodium hydride |
| NMM | N-methylmorpholine |
| NMP | 1-methyl-2-pyrrolidinone |
| NMR | Nuclear Magnetic Resonance |
| PG | Protecting group. Exemplary protecting groups include Boc, |
| | Cbz, Fmoc and benzyl |
| Pg. | Page |
| PPP | Platelet poor plasma |
| PRP | Platelet rich plasma |
| q | Quartet |
| Rpm | Revolutions per minute |
| s | Singlet |
| t | Triplet |
| TFA | trifluoroacetic acid |
| THF | Tetrahydrofuran |
| TLC | Thin layer chromatography |
| UV | Ultraviolet |
| Vol. | Volume |
| δ | Chemical shift |

The term “alkyl” refers to a linear or branched-chain saturated hydrocarbyl substituent (i.e., a substituent obtained from a hydrocarbon by removal of a hydrogen) containing from one to twenty carbon atoms; in one embodiment from one to twelve carbon atoms; in another embodiment, from one to ten carbon atoms; in another embodiment, from one to six carbon atoms; and in another embodiment, from one to four carbon atoms. Examples of such substituents include methyl, ethyl, propyl (including n-propyl and isopropyl), butyl (including n-butyl, isobutyl, sec-butyl and tert-butyl), pentyl, iso-amyl, hexyl and the like.

The term “alkenyl” refers to a linear or branched-chain hydrocarbyl substituent containing one or more double bonds and from two to twenty carbon atoms; in another embodiment, from two to twelve carbon atoms; in another embodiment, from two to six carbon atoms; and in another embodiment, from two to four carbon atoms. Examples of alkenyl include ethenyl (also known as vinyl), allyl, propenyl (including 1-propenyl and 2-propenyl) and butenyl (including 1-butenyl, 2-butenyl and 3-butenyl). The term “alkenyl” embraces substituents having “cis” and “trans” orientations, or alternatively, “E” and “Z” orientations.

The term “benzyl” refers to methyl radical substituted with phenyl, i.e., the following structure:

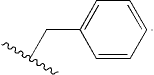

The term “carbocyclic ring” refers to a saturated cyclic, partially saturated cyclic, or aromatic ring containing from 3 to 14 carbon ring atoms (“ring atoms” are the atoms bound together to form the ring). A carbocyclic ring typically contains from 3 to 10 carbon ring atoms. Examples include cyclopropyl, cyclobutyl, cyclopentyl, cyclopentenyl, cyclopentadienyl, cyclohexyl, cyclohexenyl, cyclohexadienyl, and phenyl. A “carbocyclic ring system” alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluorenyl, and decalinyl.

The term “heterocyclic ring” refers to a saturated cyclic, partially saturated cyclic, or aromatic ring containing from 3 to 14 ring atoms (“ring atoms” are the atoms bound together to form the ring), in which at least one of the ring atoms is a heteroatom that is oxygen, nitrogen, or sulfur, with the remaining ring atoms being independently selected from the group consisting of carbon, oxygen, nitrogen, and sulfur.

The term “cycloalkyl” refers to a saturated carbocyclic substituent having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “cycloalkyl” also includes substituents that are fused to a C₆-C₁₀ aromatic ring or to a 5-10-membered heteroaromatic ring, wherein a group having such a fused cycloalkyl group as a substituent is bound to a carbon atom of the cycloalkyl group. When such a fused cycloalkyl group is substituted with one or more substituents, the one or more substitutents, unless otherwise specified, are each bound to a carbon atom of the cycloalkyl group. The fused C₆-C₁₀ aromatic ring or to a 5-10-membered heteroaromatic ring may be optionally substituted with halogen, C₁-C₆ alkyl, C₃-C₁₀ cycloalkyl, or ═O.

The term “cycloalkenyl” refers to a partially unsaturated carbocyclic substituent having three to fourteen carbon atoms, typically three to ten carbon atoms. Examples of cycloalkenyl include cyclobutenyl, cyclopentenyl, and cyclohexenyl.

A cycloalkyl or cycloalkenyl may be a single ring, which typically contains from 3 to 6 ring atoms. Examples include cyclopropyl, cyclobutyl, cyclopentyl, cyclopentenyl, cyclopentadienyl, cyclohexyl, cyclohexenyl, cyclohexadienyl, and phenyl. Alternatively, 2 or 3 rings may be fused together, such as bicyclodecanyl and decalinyl.

The term “aryl” refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” also includes substituents such as phenyl, naphthyl and anthracenyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the aryl group. When such a fused aryl group is substituted with one more substituents, the one or more substitutents, unless otherwise specified, are each bound to an aromatic carbon of the fused aryl group. The fused C₄-C₁₀ carbocyclic or 4-10-membered heterocyclic ring may be optionally substituted with halogen, C₁-C₆ alkyl, C₃-C₁₀ cycloalkyl, or ═O. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl.

In some instances, the number of carbon atoms in a hydrocarbyl substituent (e.g., alkyl, alkenyl, cycloalkyl, cycloalkenyl, aryl, etc.) is indicated by the prefix “Cₓ-C_{y}-,” wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl refers to saturated cycloalkyl containing from 3 to 6 carbon ring atoms.

In some instances, the number of atoms in a cyclic substituent containing one or more heteroatoms (e.g., heteroaryl or heterocycloalkyl) is indicated by the prefix “X—Y-membered”, wherein wherein x is the minimum and y is the maximum number of atoms forming the cyclic moiety of the substituent. Thus, for example, 5-8-membered heterocycloalkyl refers to a heterocycloalkyl containing from 5 to 8 atoms, including one ore more heteroatoms, in the cyclic moiety of the heterocycloalkyl.

The term “hydrogen” refers to hydrogen substituent, and may be depicted as —H.

The term “hydroxy” or “hydroxyl” refers to —OH. When used in combination with another term(s), the prefix “hydroxy” indicates that the substituent to which the prefix is attached is substituted with one or more hydroxy substituents. Compounds bearing a carbon to which one or more hydroxy substituents include, for example, alcohols, enols and phenol.

The term “hydroxyalkyl” refers to an alkyl that is substituted with at least one hydroxy substituent. Examples of hydroxyalkyl include hydroxymethyl, hydroxyethyl, hydroxypropyl and hydroxybutyl.

The term “nitro” means —NO₂.

The term “cyano” (also referred to as “nitrile”) means —CN, which also may be depicted:

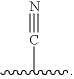

The term “carbonyl” means —C(O)—, which also may be depicted as:

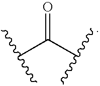

The term “amino” refers to —NH₂.

The term “alkylamino” refers to an amino group, wherein at least one alkyl chain is bonded to the amino nitrogen in place of a hydrogen atom. Examples of alkylamino substituents include monoalkylamino such as methylamino (exemplified by the formula —NH(CH₃)), which may also be depicted:

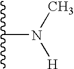

and dialkylamino such as dimethylamino, (exemplified by the formula —N(CH₃)₂, which may also be depicted:

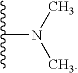

The term “aminocarbonyl” means —C(O)—NH₂, which also may be depicted as:

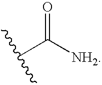

The term “halogen” refers to fluorine (which may be depicted as —F), chlorine (which may be depicted as —Cl), bromine (which may be depicted as —Br), or iodine (which may be depicted as —I). In one embodiment, the halogen is chlorine, In another embodiment, the halogen is a fluorine.

The prefix “halo” indicates that the substituent to which the prefix is attached is substituted with one or more independently selected halogen substituents. For example, haloalkyl refers to an alkyl that is substituted with at least one halogen substituent. Where more than one hydrogen is replaced with halogens, the halogens may be the identical or different. Examples of haloalkyls include chloromethyl, dichloromethyl, difluorochloromethyl, dichlorofluoromethyl, trichloromethyl, 1-bromoethyl, fluoromethyl, difluoromethyl, trifluoromethyl, 2,2,2-trifluoroethyl, difluoroethyl, pentafluoroethyl, difluoropropyl, dichloropropyl, and heptafluoropropyl. Illustrating further, “haloalkoxy” refers to an alkoxy that is substituted with at least one halogen substituent. Examples of haloalkoxy substituents include chloromethoxy, 1-bromoethoxy, fluoromethoxy, difluoromethoxy, trifluoromethoxy (also known as “perfluoromethyloxy”), and 2,2,2-trifluoroethoxy. It should be recognized that if a substituent is substituted by more than one halogen substituent, those halogen substituents may be identical or different (unless otherwise stated).

The prefix “perhalo” indicates that each hydrogen substituent on the substituent to which the prefix is attached is replaced with an independently selected halogen substituent. If all the halogen substituents are identical, the prefix may identify the halogen substituent. Thus, for example, the term “perfluoro” means that every hydrogen substituent on the substituent to which the prefix is attached is replaced with a fluorine substituent. To illustrate, the term “perfluoroalkyl” refers to an alkyl substituent wherein a fluorine substituent is in the place of each hydrogen substituent. Examples of perfluoroaIkyl substituents include trifluoromethyl (—CF₃), perfluorobutyl, perfluoroisopropyl, perfluorododecyl, and perfluorodecyl. To illustrate further, the term “perfluoroalkoxy” refers to an alkoxy substituent wherein each hydrogen substituent is replaced with a fluorine substituent. Examples of perfluoroalkoxy substituents include trifluoromethoxy (—O—CF₃), perfluorobutoxy, perfluoroisopropoxy, perfluorododecoxy, and perfluorodecoxy.

The term “oxo” refers to ═O.

The term “oxy” refers to an ether substituent, and may be depicted as —O—.

The term “alkoxy” refers to an alkyl linked to an oxygen, which may also be represented as

—O—R, wherein the R represents the alkyl group, Examples of alkoxy include methoxy, ethoxy, propoxy and butoxy.

The term “alkoxycarbonyl” means —C(O)—O-alkyl. For example, “ethoxycarbonyl” may be depicted as:

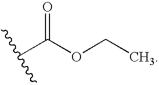

Examples of other alkoxycarbonyl include methoxycarbonyl, ethoxycarbonyl, propoxycarbonyl, butoxycarbonyl, pentoxycarbonyl, and hexyloxycarbonyl. In another embodiment, where the carbon atom of the carbonyl is attached to a carbon atom of a second alkyl, the resulting functional group is an ester.

The terms “thia” and “thia” mean a divalent sulfur atom and such a substituent may be depicted as —S—. For example, a thioether is represented as “alkyl-thio-alkyl” or, alternatively, alkyl-S-alkyl.

The term “sulfonyl” refers to —S(O)₂—, which also may be depicted as:

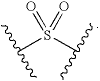

Thus, for example, “alkyl-sulfonyl-alkyl” refers to alkyl-S(O)₂-alkyl. Examples of alkylsulfonyl include methylsulfonyl, ethylsulfonyl, and propylsulfonyl.

The term “heterocycloalkyl” refers to a saturated or partially saturated ring structure containing a total of 3 to 14 ring atoms. At least one of the ring atoms is a heteroatom (i.e., oxygen nitrogen, or sulfur), with the remaining ring atoms being independently selected from the group consisting of carbon, oxygen, nitrogen, and sulfur. A heterocycloalkyl alternatively may comprise 2 or 3 rings fused together, wherein at least one such ring contains a heteroatom as a ring atom (e.g., nitrogen, oxygen, or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom.

The term “heterocycloalkyl” also includes substituents that are fused to a C₆-C₁₀ aromatic ring or to a 5-10-membered heteroaromatic ring, wherein a group having such a fused heterocycloalkyl group as a substituent is bound to a heteroatom of the heterocyclocalkyl group or to a carbon atom of the heterocycloalkyl group. When such a fused heterocycloalkyl group is substituted with one more substituents, the one or more substitutents, unless otherwise specified, are each bound to a heteroatom of the heterocyclocalkyl group or to a carbon atom of the heterocycloalkyl group. The fused C₆-C₁₀ aromatic ring or 5-10-membered heteroaromatic ring may be optionally substituted with halogen, C₁-C₆ alkyl, C₃-C₁₀ cycloalkyl, C₁-C₆ alkoxy, or ═O.

The term “heteroaryl” refers to an aromatic ring structure containing from 5 to 14 ring atoms in which at least one of the ring atoms is a heteroatom (i.e., oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. The term “heteroaryl” also includes pyridyl N-oxides and groups containing a pyridine N-oxide ring.

Examples of single-ring heteroaryls include furanyl, dihydrofuranyl, tetradydrofuranyl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiaodiazolyl, oxathiazolyl, oxadiazolyl (including oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl” or “pyrimidyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1 2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

Examples of 2-fused-ring heteroaryls include, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

Examples of 3-fused-ring heteroaryls or heterocycloalkyls include 5,6-dihydro-4H-imidazo[4,5,1-ij]quinoline, 4,5-dihydroimidazo[4,5,1-hi]indole, 4,5,6,7-tetrahydroimidazo[4,5,1-jk][1]benzazepine, and dibenzofuranyl.

Other examples of fused-ring heteroaryls include benzo-fused heteroaryls such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known a as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl,” “thionaphthenyl,” or “benzothiofuranyl”), isobenzothienyl (also known as “isobenzothiophenyl,” “isothionaphthenyl,” or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl, 2,3,1-benzoxazinyl, or 3,1,4-benzoxazinyl), benzisoxazinyl (including 1,2-benzisoxazinyl or 1,4-benzisoxazinyl), tetrahydroisoquinolinyl, carbazolyl, xanthenyl, and acridinyl.

The term “heteroaryl” also includes substituents such as pyridyl and quinolinyl that are fused to a C₄-C₁₀ carbocyclic ring, such as a C₅ or a C₆ carbocyclic ring, or to a 4-10-membered heterocyclic ring, wherein a group having such a fused aryl group as a substituent is bound to an aromatic carbon of the heteroaryl group or to a heteroatom of the heteroaryl group. When such a fused heteroaryl group is substituted with one more substituents, the one or more substitutents, unless otherwise specified, are each bound to an aromatic carbon of the heteroaryl group or to a heteroatom of the heteroaryl group. The fused C₄-C₁₀ carbocyclic or 4-10-membered heterocyclic ring may be optionally substituted with halogen, C₁-C₆ alkyl, C₃-C₁₀ cycloalkyl, or ═O.

A substituent is “substitutable” if it comprises at least one carbon, sulfur, oxygen or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon, oxygen, sulfur or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R,″ wherein R′ and R″ together with the nitrogen atom to which they are attached, may form a heterocyclic ring. The heterocyclic ring formed from R′ and R″ together with the nitrogen atom to which they are attached may be partially or fully saturated. In one embodiment, the heterocyclic ring consists of 3 to 7 atoms. In another embodiment, the heterocyclic ring is selected from the group consisting of pyrrolyl, imidazolyl, pyrazolyl, triazolyl, tetrazolyl, isoxazolyl, pyridyl and thiazolyl.

This specification uses the terms “substituent,” “radical,” and “group” interchangeably.

If a group of substituents are collectively described as being optionally substituted by one or more of a list of substituents, the group may include: (1) unsubstitutable substituents, (2) substitutable substituents that are not substituted by the optional substituents, and/or (3) substitutable substituents that are substituted by one or more of the optional substituents.

If a substituent is described as being optionally substituted with up to a particular number of non-hydrogen substituents, that substituent may be either (1) not substituted; or (2) substituted by up to that particular number of non-hydrogen substituents or by up to the maximum number of substitutable positions on the substituent, whichever is less. Thus, for example, if a substituent is described as a heteroaryl optionally substituted with up to 3 non-hydrogen substituents, then any heteroaryl with less than 3 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent. To illustrate further, if an amino nitrogen is described as being optionally substituted with up to 2 non-hydrogen substituents, then the nitrogen will be optionally substituted with up to 2 non-hydrogen substituents if the amino nitrogen is a primary nitrogen, whereas the amino nitrogen will be optionally substituted with up to only 1 non-hydrogen substituent if the amino nitrogen is a secondary nitrogen.

A prefix attached to a multi-moiety substituent only applies to the first moiety. To illustrate, the term “alkylcycloalkyl” contains two moieties: alkyl and cycloalkyl. Thus, a C₁-C₆-prefix on C₁-C₆-alkylcycloalkyl means that the alkyl moiety of the alkylcycloalkyl contains from 1 to 6 carbon atoms; the C₁-C₆-prefix does not describe the cycloalkyl moiety. To illustrate further, the prefix “halo” on haloalkoxyalkyl indicates that only the alkoxy moiety of the alkoxyalkyl substituent is substituted with one or more halogen substituents. If the halogen substitution may only occur on the alkyl moiety, the substituent would be described as “alkoxyhaloalkyl.” If the halogen substitution may occur on both the alkyl moiety and the alkoxy moeity, the substituent would be described as “haloalkoxyhaloalkyl.”

When a substituent is comprised of multiple moieties, unless otherwise indicated, it is the intention for the final moiety to serve as the point of attachment to the remainder of the molecule. For example, in a substituent A-B-C, moiety C is attached to the remainder of the molecule. In a substituent A-B-C-D, moiety D is attached to the remainder of the molecule. Similarly, in a substituent aminocarbonylmethyl, the methyl moiety is attached to the remainder of the molecule, where the substituent may also be be depicted as

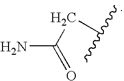

In a substituent trifluoromethylaminocarbonyl, the carbonyl moiety is attached to the remainder of the molecule, where the substituent may also be depicted as

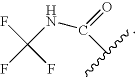

If substituents are described as being “independently selected” from a group, each substituent is selected independent of the other. Each substituent therefore may be identical to or different from the other substituent(s).

### Isomers

When an asymmetric center is present in a compound of formula I (hereinafter understood to mean formula I, I′, I″ or I′″), hereinafter referred to as the compound of the invention, the compound may exist in the form of optical isomers (enantiomers). In one embodiment, the present invention comprises enantiomers and mixtures, including racemic mixtures of the compounds of formula I. In another embodiment, for compounds of formula I that contain more than one asymmetric center, the present invention comprises diastereomeric forms (individual diastereomers and mixtures thereof) of compounds. When a compound of formula I contains an alkenyl group or moiety, geometric isomers may arise.

### Tautomeric Forms

The present invention comprises the tautomeric forms of compounds of formula I. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound.

### Salts

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclylic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), arylalkyl halides (e.g., benzyl and phenethyl bromides), and others.

In one embodiment, hemisalts of acids and bases may also be formed, for example, hemisulphate and hemicalcium salts.

### Prodrugs

Also within the scope of the present invention are so-called “prodrugs” of the compound of the invention. Thus, certain derivatives of the compound of the invention which may have little or no pharmacological activity themselves can, when administered into or onto the body, be converted into the compound of the invention having the desired activity, for example, by hydrolytic cleavage. Such derivatives are referred to as “prodrugs.” Further information on the use of prodrugs may be found in “Pro-drugs as Novel Delivery Systems, Vol. 14, ACS Symposium Series (T Higuchi and W Stella) and “Bioreversible Carriers in Drug Design,” Pergamon Press, 1987 (ed. E B Roche, American Pharmaceutical Association). Prodrugs in accordance with the invention can, for example, be produced by replacing appropriate functionalities present in the compounds of any of formula I with certain moieties known to those skilled in the art as “pro-moieties” as described, for example, in “Design of Prodrugs” by H Bundgaard (Elsevier, 1985).

### Isotopes

The present invention also includes isotopically labelled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

### Administration and Dosing

Typically, a compound of the invention is administered in an amount effective to treat or prevent a condition as described herein. The compounds of the invention are administered by any suitable route in the form of a pharmaceutical composition adapted to such a route, and in a dose effective for the treatment or prevention intended. Therapeutically effective doses of the compounds required to treat or prevent the progress of the medical condition are readily ascertained by one of ordinary skill in the art using preclinical and clinical approaches familiar to the medicinal arts.

The compounds of the invention may be administered orally. Oral administration may involve swallowing, so that the compound enters the gastrointestinal tract, or buccal or sublingual administration may be employed by which the compound enters the blood stream directly from the mouth.

In another embodiment, the compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment or prevention of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

### Use in the Preparation of a Medicament

In another embodiment, the invention comprises the use of one or more compounds of the invention for the preparation of a medicament for the treatment or prevention of the conditions recited herein.

### Pharmaceutical Compositions

For the treatment or prevention of the conditions referred to above, the compound of the invention can be administered as compound per se. Alternatively, pharmaceutically acceptable salts are suitable for medical applications because of their greater aqueous solubility relative to the parent compound.

In another embodiment, the present invention comprises pharmaceutical compositions. Such pharmaceutical compositions comprise a compound of the invention presented with a pharmaceutically-acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The compounds of the present invention may be administered by any suitable route, preferably in the form of a pharmaceutical composition adapted to such a route, and in a dose effective for the treatment or prevention intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of formula I are ordinarily combined with one or more adjuvants. Such capsules or tablets may contain a controlled-release formulation. In the case of capsules, tablets, and pills, the dosage forms also may comprise buffering agentsor may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneally; intramuscular injections, intrasternal injections, and infusion. Injectable preparations (e.g., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Formulations suitable for topical administration to the eye include, for example, eye drops wherein the compound of this invention is dissolved or suspended in suitable carrier. A typical formulation suitable for ocular or aural administration may be in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

### Co-Administration

The compounds of the present invention can be used, alone or in combination with other therapeutic agents, in the treatment or prevention of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### Kits

The present invention further comprises kits that are suitable for use in performing the methods of treatment or prevention described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Intermediates

In another embodiment, the invention relates to the novel intermediates useful for preparing the compounds of the invention

### General Synthetic Schemes

The compounds of the formula I may be prepared by the methods described below, together with synthetic methods known in the art of organic chemistry, or modifications and derivatisations that are familiar to those of ordinary skill in the art. The starting materials used herein are commercially available or may be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999, which are hereby incorporated by reference.

Compounds of formula I, or their pharmaceutically acceptable salts, can be prepared according to the reaction Schemes discussed hereinbelow. Unless otherwise indicated, the substituents in the Schemes are defined as above. Isolation and purification of the products is accomplished by standard procedures, which are known to a chemist of ordinary skill.

The following schemes are exemplary of the processes for making compounds of formula I.

Unless otherwise specified, the stereochemistry of the structures shown in the schemes is intended to denote relative stereochemistry, rather than absolute stereochemistry. As an illustrative, non-limiting example, in Scheme A below, the structure

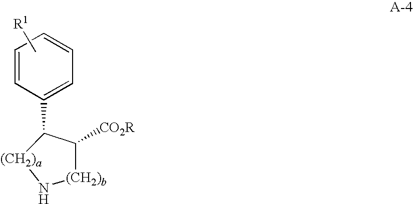

is intended to denote that the R1-substituted phenyl group and the CO₂R group have a cis stereochemistry. Both of the enantiomers having the two groups in such cis stereochemistry are intended to be encompassed by the structure.

General Synthetic Schemes—AMPA

As appreciated by the artisan, the use of formula I is a convenience and the invention is understood to include each and every species falling thereunder as though individually set forth herein. Thus the invention contemplates each species separately and any and all conbinations of such species.

Without restriction, the compound of the invention can be prepared by one of ordinary skill in the art following art recognized techniques and procedures. More specifically, compounds of formula I can be prepared as set forth in the schemes, methods, and examples set forth below. It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

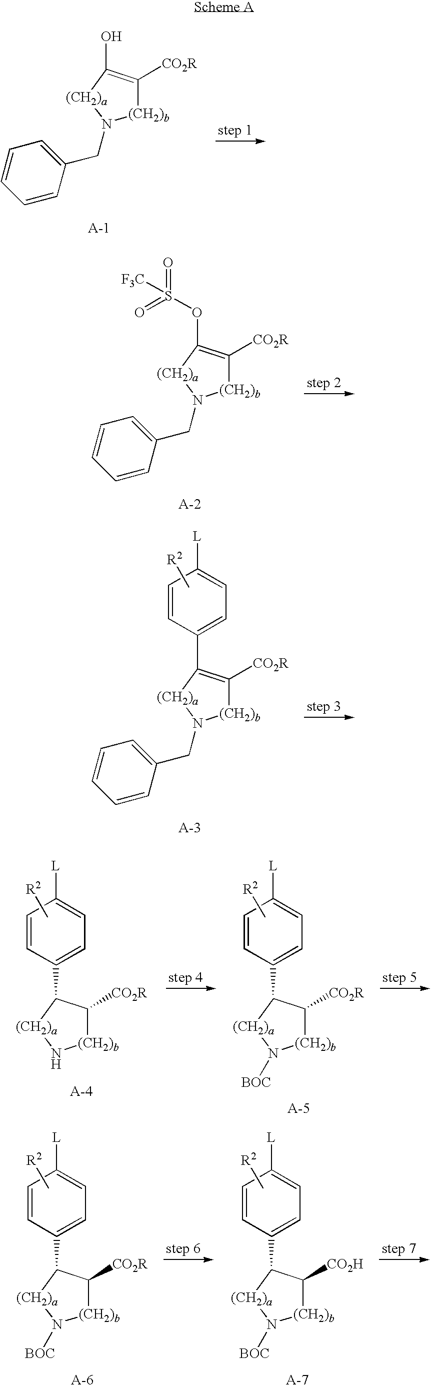

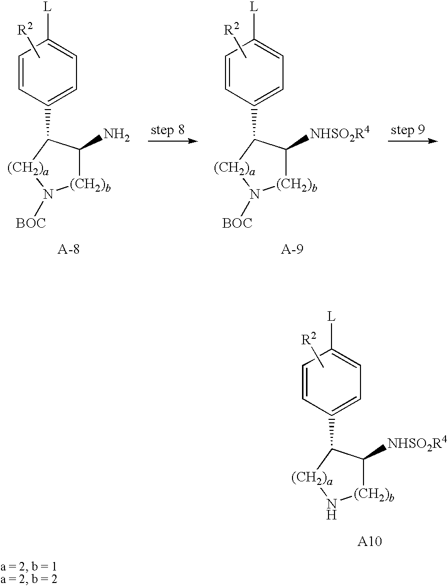

Scheme A depicts a method for the synthesis of trans piperidine compounds of formula I.

In step 1, for example, the ketoester of structure A-1 can be treated with a base such as sodium hydride in diethyl ether, followed by treatment with drifluoromethanesulfonic anhydride to provide the vinyl triflate of structure A-2. Other non-limiting examples of bases which can be used include hindered amine bases such as triethylamine, diisopropylethylamine, 2,6-lutidine or 2,6-di-tert-butyl-4-methyl pyridine in a suitable solvent, such as dichloromethane.

In step 2, for example, the vinyl triflate of structure A-2 can be coupled to a suitable aryl boronic acid of structure ArB(OH)₂, wherein Ar represents a suitable aryl group, under standard palladium catalyzed cross-coupling reaction conditions well known to one of ordinary skill in the art to provide the compound of structure A-3. [Suzuki, A., Journal of Organometallic Chemistry, 576, 147-169 (1999), Miyaura and Suzuki, Chemical Reviews, 95, 2457-2483 (1995).] More specifically, the vinyl triflate A-2 is combined with 1 to 3 equivalents of aryl boronic acid and a suitable base, such as 2 to 5 equivalents of potassium carbonate, in a suitable organic solvent such as THF. A palladium catalyst is added, such as 0.02 equivalents of palladium tetrakistripheylphosphine, and the reaction mixture is heated to temperatures ranging from 60 to 100° C. for 1 to 24 hours. The reaction is not limited to the employment of this solvent, base, or catalyst as many other conditions may be used.

In step 3, the resultant unsaturated ring of compound A-3 can be reduced by treatment with a palladium catalyst, such as 10% Pd/C, and hydrogen gas at elevated pressure such as 50 psi in a suitable solvent such as ethanol, or a solvent mixture such as ethanol and acetic acid. Hydrogenation also serves to remove the benzyl protecting group to afford the cis piperidine A-4.

In step 4, the free amine group of compound A-4 can be protected with, for example, a BOC group by treatment with a base, such as potassium carbonate, and di-tert-butyl dicarbonate in a solvent such as THF to afford the BOC piperidine A-5.

In step 5, the cis-piperidine compound A-5 can be epimerized by treatment with a base, such as sodium ethoxide, using a suitable solvent and temperature, such as ethanol at reflux, to afford the trans-piperidine ester A-6.

In step 6, the ester of compound A-6 can be converted to the carboxylic acid A-7 under conditions well known in the art. For example, the ester A-6 can be treated with excess lithium-, sodium-, or potassium-hydroxide in a suitable solvent such as a mixture of water and methanol, or water, alcohol and THF, at elevated temperatures if necessary. An acidic workup can afford the carboxylic acid A-7.

In step 7, the carboxylic acid functionality of compound A-7 can be converted into the primary amine via the Curtius rearrangement under conditions well known in the art. For example, the carboxylic acid A-7 can be treated with diphenylphosphoryl azide (DPPA) in a suitable solvent such as toluene at elevated temperatures such as 80° C. An organic base such as triethylamine may be added. The crude isocyanate intermediate subsequently may be hydrolyzed using, for example, aqueous hydroxide in combination with an organic solvent such as THF. Alternatively, the isocyanate may be trapped with an organic alcohol such as t-butanol to afford the analogous carbamate. A preferred method involves the treatment of the crude isocyanate with 2 M sodium hydroxide in THF to afford the amine A-8.

In step 8, the amino functionality of compound A-8 can be converted into the sulfonamide under conditions well known in the art. For example, a mixture of the amine A-8 and a suitable base such as triethylamine or 1,8-diazabicyclo[5.4.0]undec-7-ene (DBU) can be treated with a sulfonyl chloride in a suitable solvent such as dichloromethane or DMF. Cooling temperatures may be used, such as 0° C.

In step 9, the BOC group of compound A-9 can be cleaved using known methods to afford the amine product A-10. For example, a strong acid such as HCl, H₂SO₄, or TFA may be added to the compound A-9 in a suitable solvent such as ether or dioxane, or alcohols such as methanol or ethanol.

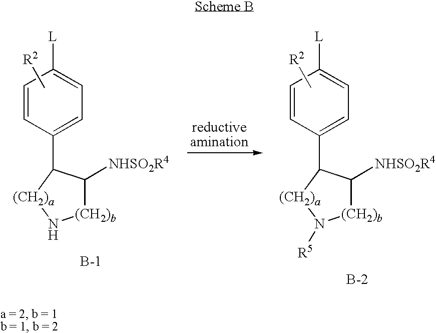

In Scheme B, the free amine having structure B-1 is alkylated via reductive alkylation using conditions well known in the art to yield the compound of structure B-2. For example, the amine can be treated with an aldehyde or a ketone and a reducing agent such as sodium cyanoborohydride, sodium hydride, or sodium- or tetramethylammonium-triacetoxyborohydride, in a suitable solvent such as dichloromethane, DCE, THF, ether, or toluene.

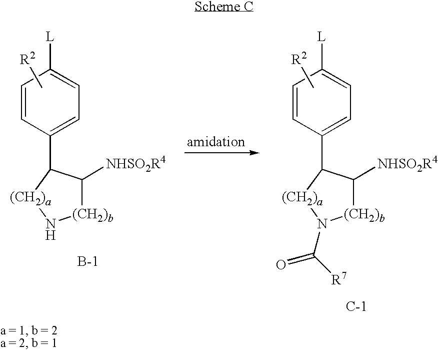

In Scheme C, the free amine having structure B-1 is converted to the amide using conditions well known in the art to yield the compound of structure C-1. For example, the amine can be dissolved in a solvent such as THF or dichloromethane, and treated with a suitable base such as triethylamine, pyridine, diisopropylethylamine followed by an acid chloride of is structure [R—COCl]. Alternatively, the acylating agent might be an acid anhydride [R—CO—O—CO—R]. Alternatively, the amine B-1 may be treated with a carboxylic acid RCO₂H in the presence of a coupling agent such as HBTU using methods that are well known in the art to afford the amide product C-1.

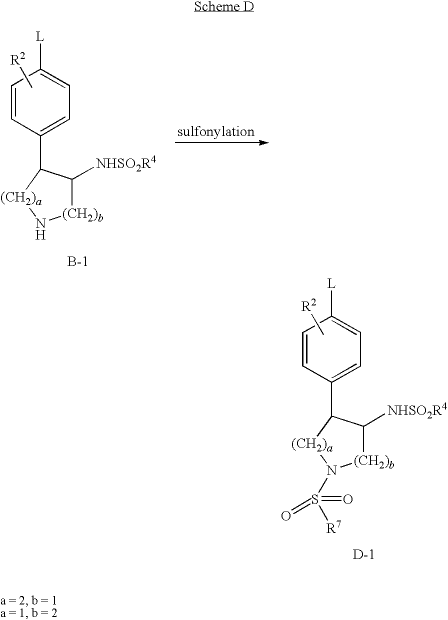

In Scheme D, the free amine having structure B-1 is converted to the sulfonamide using conditions well known in the art to yield the compound of structure D-1. For example, the amine can be dissolved in a solvent such as dichloromethane and treated with a base such as triethylamine or pyridine, followed by treatment with a sulfonyl chloride R—SO₂Cl to afford the sulfonamide product D-1.

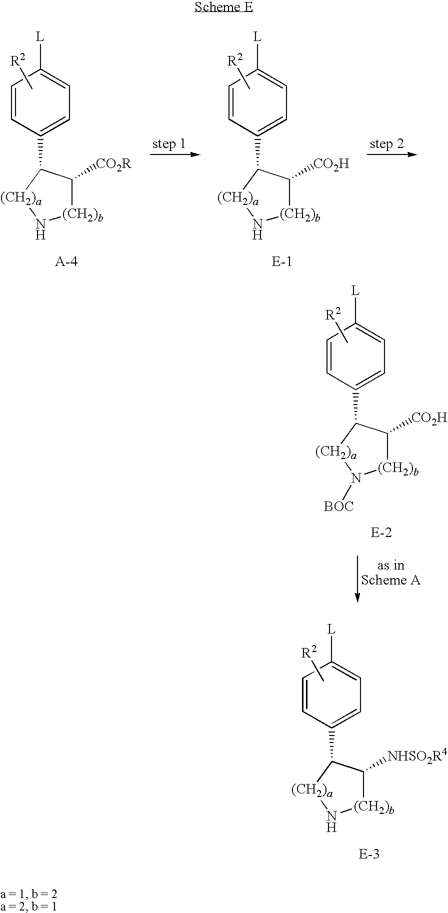

Scheme E depicts a method for the synthesis of cis piperidine compounds of formula I.

In step 1, the cis ester of structure A-4 can be converted to the carboxylic acid E-1 under conditions well known in the art. For example, the ester A-4 may be treated with a strong acid such as HCl in water and heated to afford the hydrolyzed carboxylic acid E-1 with retention of relative stereochemistry.

In step 2, the free amine group of compound E-1 can be protected with, for example, a BOC group by treatment with a base, such as potassium carbonate, and di-tert-butyl dicarbonate in a solvent such as THF to afford the BOC piperidine E-2.

The formation of the cis piperidine sulfonamide product E-3 was performed according to the general methods described within Scheme A, steps 7 to 9.

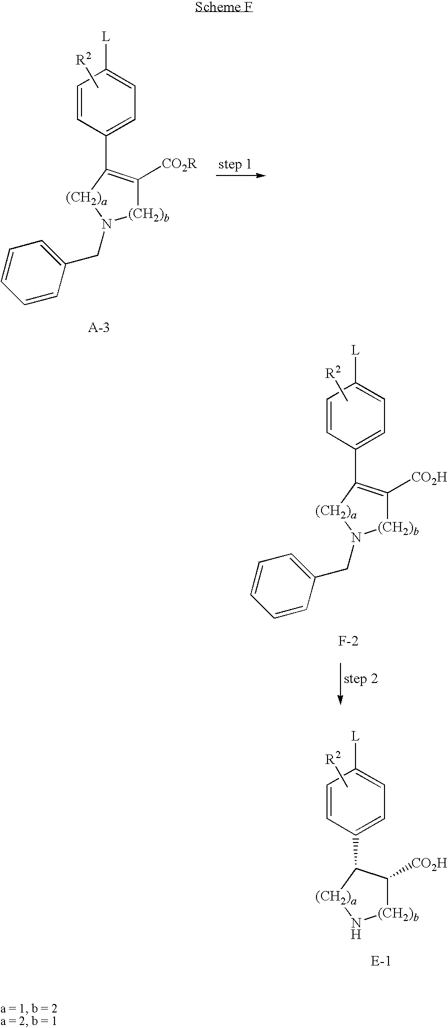

Scheme F depicts an alternative method for the synthesis of cis piperidine carboxylic acid of formula E-1.

In step 1, the ester of compound A-3 can be converted to the carboxylic acid F-2 under conditions well known in the art. For example, the ester A-3 can be treated with excess lithium-, sodium-, or potassium-hydroxide in a suitable solvent such as a mixture of water and methanol, or water, alcohol and THF, at elevated temperatures if necessary. An acidic workup can afford the carboxylic acid F-2.

In step 2, the unsaturated ring of compound F-2 can be reduced by treatment with a catalyst, such as platium oxide, and hydrogen gas at elevated pressure such as 50 psi in a suitable solvent such as ethanol, or a solvent mixture such as THF and water. Elevated temperatures, such as 40-50° C. may be used. Hydrogenation also serves to remove the benzyl protecting group to afford the cis piperidine E-1.

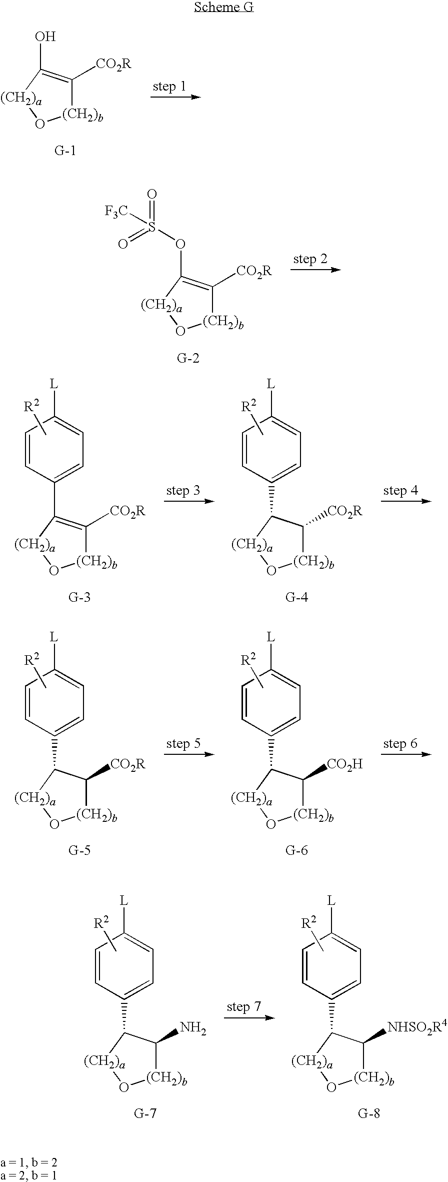

Scheme G depicts a method for the synthesis of trans tetrahydropyran compounds of formula I.

In step 1, for example, the ketoester of structure G-1 can be treated with a base such as sodium hydride in diethyl ether, followed by treatment with drifluoromethanesulfonic anhydride to provide the vinyl triflate of structure G-2. Other non-limiting examples of bases which can be used include hindered amine bases such as triethylamine, diisopropylethylamine, 2,6-lutidine or 2,6-di-tert-butyl-4-methyl pyridine in a suitable solvent, such as dichloromethane.

In step 2, for example, the vinyl triflate of structure G-2 can be coupled to a suitable aryl boronic acid of structure ArB(OH)₂, wherein Ar represents a suitable aryl group, under standard palladium catalyzed cross-coupling reaction conditions well known to one of ordinary skill in the art to provide the compound of structure G-3. [Suzuki, A., Journal of Organometallic Chemistry, 576, 147-169 (1999), Miyaura and Suzuki, Chemical Reviews, 95, 2457-2483 (1995).] More specifically, the vinyl triflate G-2 is combined with 1 to 3 equivalents of aryl boronic acid and a suitable base, such as 2 to 5 equivalents of potassium carbonate, in a suitable organic solvent such as THF. A palladium catalyst is added, such as 0.02 equivalents of palladium tetrakistripheylphosphine, and the reaction mixture is heated to temperatures ranging from 60 to 100° C. for 1 to 24 hours. The reaction is not limited to the employment of this solvent, base, or catalyst as many other conditions may be used.

In step 3, the resultant unsaturated ring of compound G-3 can be reduced under conditions well known in the art. For example, treatment of G-3 with a palladium catalyst, such as 10% Pd/C, and hydrogen gas at elevated pressure such as 50 psi in a suitable solvent such as ethanol, methanol, or ethyl acetate afford the cis tetrahydropyran product G-4.

In step 4, the cis-tetrahydropyrane compound G-4 can be epimerized by treatment with a base, such as sodium ethoxide, using a suitable solvent and temperature, such as ethanol at reflux, to afford the trans-tetrahydropyran ester G-5.

In step 5, the ester of compound G-5 can be converted to the carboxylic acid G-6 under conditions well known in the art. For example, the ester G-5 can be treated with excess lithium-, sodium-, or potassium-hydroxide in a suitable solvent such as a mixture of water and methanol, or water, alcohol and THF, at elevated temperatures if necessary. An acidic workup can afford the carboxylic acid G-6.

In step 7, the carboxylic acid functionality of compound G-6 can be converted into the primary amine via the Curtius rearrangement under conditions well known in the art. For example, the carboxylic acid G-6 can be treated with diphenylphosphoryl azide in a suitable solvent such as toluene at elevated temperatures such as 80° C. An organic base such as triethylamine may be added. The crude isocyanate intermediate subsequently may be hydrolyzed using, for example, aqueous hydroxide in combination with an organic solvent such as THF. Alternatively, the isocyanate may be trapped with an organic alcohol such as t-butanol to afford the analogous carbamate. A preferred method involves the treatment of the crude isocyanate with 2 M sodium hydroxide in THF to afford the amine G-7.

In step 8, the amino functionality of compound G-7 can be converted into the sulfonamide G-8 under conditions well known in the art. For example, a mixture of the amine G-7 and a suitable base such as triethylamine or 1,8-diazabicyclo[5.4.0]undec-7-ene can be treated with a sulfonyl chloride in a suitable solvent such as dichloromethane or DMF. Cooling temperatures may be used, such as 0° C.

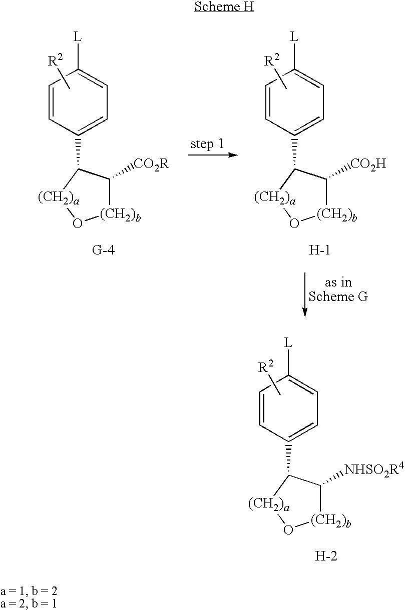

Scheme H depicts a method for the synthesis of cis tetrahydropyran compounds of formula I.

In step 1, the cis ester of structure G-4 can be converted to the carboxylic acid H-1 under conditions well known in the art. For example, the ester G-4 may be treated with a strong acid such as HCl in water and heated to afford the hydrolyzed carboxylic acid H-1 with retention of relative stereochemistry.

The formation of the cis tetrahydropyran sulfonamide product H-2 was performed according to the general methods described within Scheme G, steps 6 to 7.

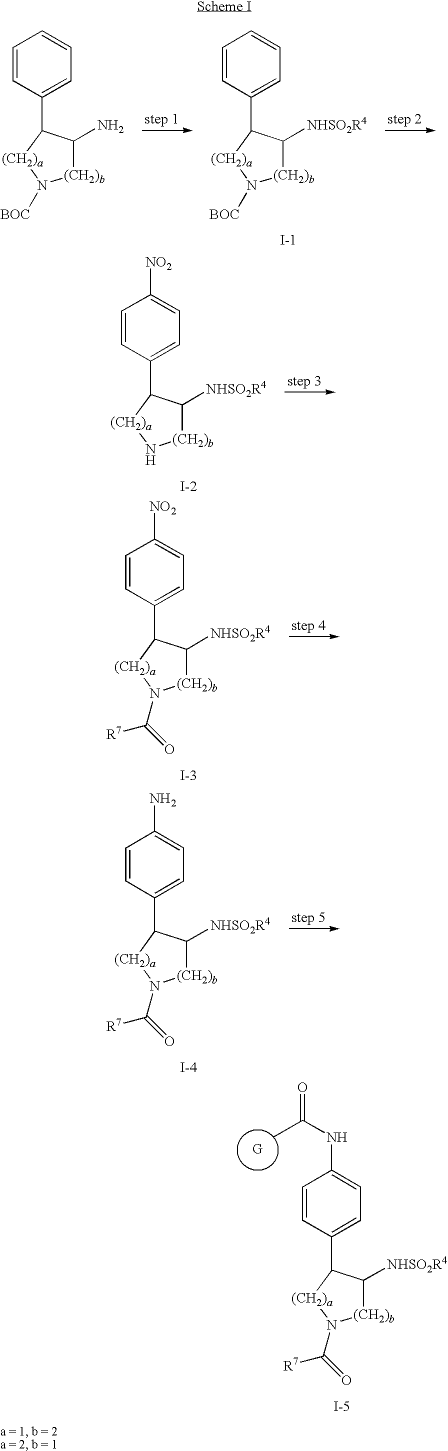

Scheme I depicts a method for the synthesis of cis or trans piperidine compounds of formula I.

In step 1, the amino functionality of a compound such as A-8 can be converted into the sulfonamide I-1 under conditions well known in the art. For example, a mixture of the amine such as A-8 (R2, L=H) and a suitable base such as triethylamine or 1,8-diazabicyclo[5.4.0]undec-7-ene can be treated with a sulfonyl chloride in a suitable solvent such as dichloromethane or DMF. Cooling temperatures may be used, such as 0° C., to afford the sulfonamide product I-1.

In step 2, the phenyl ring may undergo nitration according to conditions well known in the art. For example, treatment of I-1 in a solvent such as nitromethane with nitric acid in the presence of a strong acid such as sulfuric acid, with cooling at temperatures such as 0° C. will effect the removal of the BOC protecting group and afford the nitrobenzene compound I-2.

In step 3,the free amine having structure I-2 is converted to the amide using conditions well known in the art to yield the compound of structure I-3. For example, the amine can be dissolved in a solvent such as THF or dichloromethane, and treated with a suitable base such as triethylamine, pyridine, diisopropylethylamine followed by an acid chloride of structure [R—COCl]. Alternatively, the acylating agent might be an acid anhydride [R—CO—O—CO—R]. Alternatively, the amine B-1 may be treated with a carboxylic acid RCO₂H in the presence of a coupling agent such as HBTU using methods that are welt known in the art to afford the amide product I-3.

In step 4, the nitro group may be reduced according to conditions well known in the art. For example, treatment of I-3 with a palladium catalyst, such as 10% Pd/C, and hydrogen gas at elevated pressure such as 50 psi in a suitable solvent such as ethanol, methanol, or ethyl acetate afford the aniline product I-4.

In step 5, the free amine having structure I-4 is converted to the amide using conditions well known in the art to yield the compound of structure I-5. For example, the amine can be dissolved in a solvent such as THF or dichloromethane, and treated with a suitable base such as triethylamine, pyridine, diisopropylethylamine followed by an acid chloride of structure [R—COCl]. Alternatively, the acylating agent might be an acid anhydride [R—CO—O—CO—R]. Alternatively, the amine B-1 may be treated with a carboxylic acid RCO₂H in the presence of a coupling agent such as HBTU using methods that are well known in the art to afford the amide product I-5.

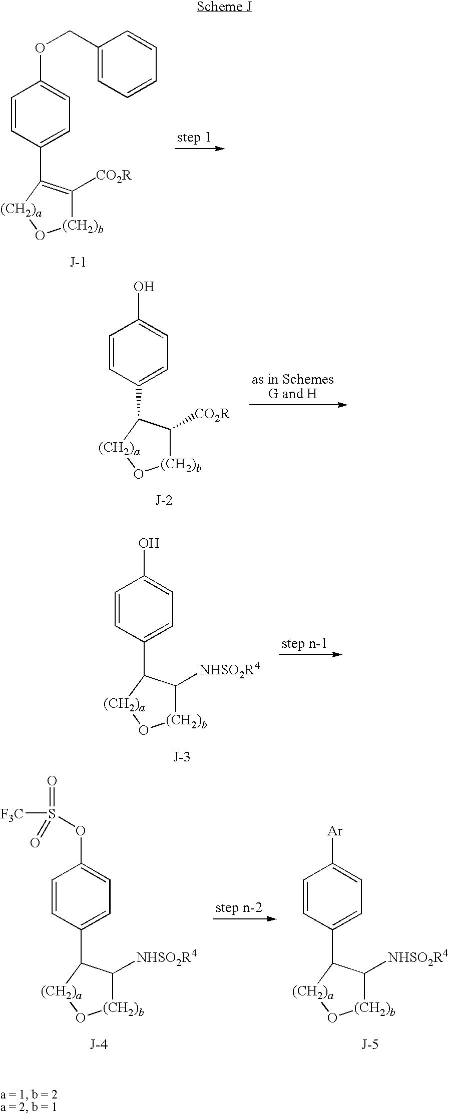

In Scheme J, the compounds of formulas J-1 through J-3 can be prepared in a manner analagous to the procedures set forth in Schemes G and H.

In step n-1, for example, the hydroxyl of structure J-3 can be treated with a base such as 2,6-lutidine in dichloromethane, followed by treatment with drifluoromethanesulfonic anhydride to provide the aryl triflate of structure J-4. Other non-limiting examples of bases which can be used include hindered amine bases such as triethylamine, diisopropylethylamine, or 2,6-di-tert-butyl-4-methyl pyridine in a suitable solvent, such as dichloroethane or THF. An acylation catalyst such as 4-dimethylaminopyridine may be used.

In step n-2, for example, the aryl triflate of structure J-4 can be coupled to an aryl boronic acid of structure ArB(OH)₂, wherein Ar represents a suitable aryl group, under standard palladium catalyzed cross-coupling reaction conditions will known to one of ordinary skill in the art to provide the compound of structure J-5. [Suzuki, A., Journal of Organometallic Chemistry, 576, 147-169 (1999), Miyaura and Suzuki, Chemical Reviews, 95, 2457-2483 (1995).] More specifically, the aryl triflate J-4 is combined with 1 to 3 equivalents of aryl boronic acid and a suitable base, such as 2 to 5 equivalents of potassium phosphate, in a suitable organic solvent such as THF or dioxane. Potassium bromide may also be included. A palladium catalyst is added, such as 0.02 equivalents of palladium tetrakistripheylphosphine, and the reaction mixture is heated to temperatures ranging from 60 to 100° C. for 1 to 24 hours. The reaction is not limited to the employment of this solvent, base, or catalyst as many other conditions may be used.

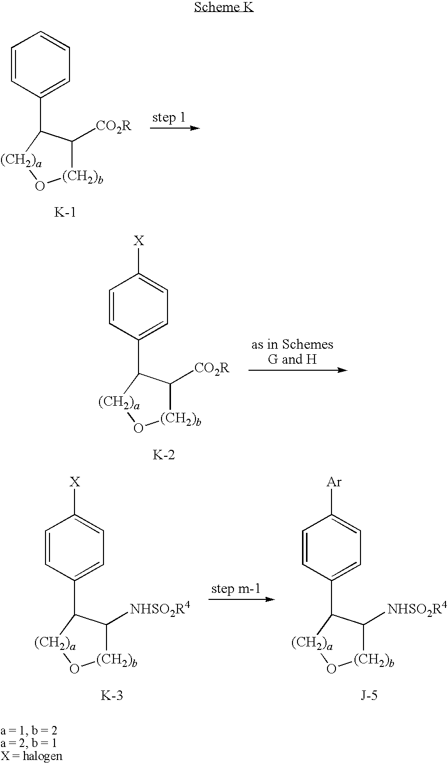

Scheme K depicts an alternative method for the synthesis of a compound of formula J-5. The tetrahydropyran compound of formula K-1 can be prepared in a manner analagous to the procedures set forth in Schemes G and H.

In step 1, the compound of formula K-1 may be iodinated under standard conditions well known to one skilled in the art. For example, K-1 may be treated with iodination conditions such as iodine and bis(trifluoroacetoxy)iodobenzene in a solvent such as dichloromethane, chloroform or carbontetrachloride. Alternatively, iodination may be conducted under acidic conditions such as iodine in a mixture of nitric acid and sulfuric acid. The resulting iodide K-2 may be converted to sulfonamide K-3 in a manner analogous to the procedure set forth in Schemes G and H.

In step m-1, the compound of formula K-3 is coupled to a suitable aryl boronic acid in a manner analogous to the procedure set forth in Scheme J. step n-2. Alternatively, compounds of formula K-3 may be treated with, for example, bis(pinacolato)diboron, a suitable catalyst such as PdCl₂(dppf), a base such as potassium acetate, while heating to 80 to 140° C. in a suitable solvent such as DMF or DMSO to afford the corresponding boronate. Subsequent treatment with standard palladium cross-coupling conditions with a suitable aryl bromide, iodide, or triflate can afford the desired product J-5.

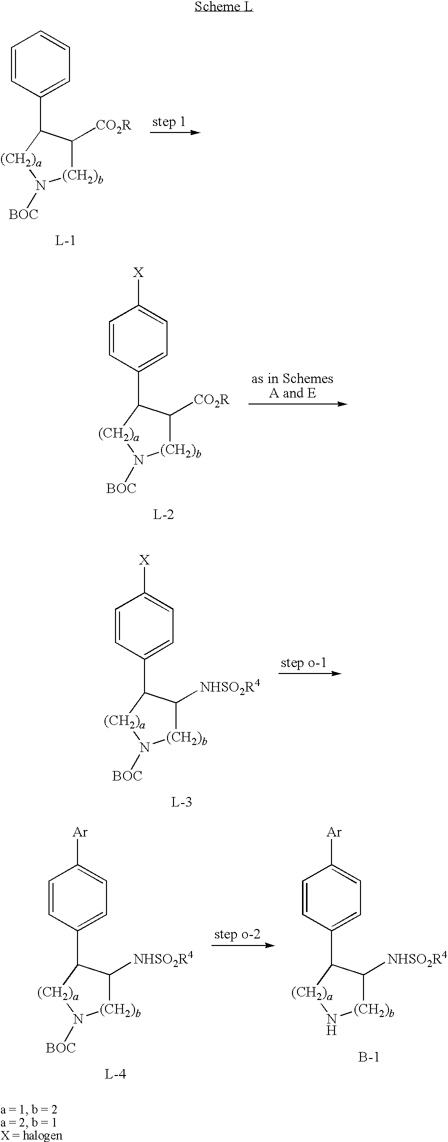

Scheme L depicts an alternative method for the synthesis of a compound of formula B-1. The piperidine compound of formula L-1 can be prepared in a manner analagous to the procedures set forth in Schemes A and E.

In step 1, the compound of formula L-1 may be iodinated under standard conditions well known to one skilled in the art. For example, L-1 may be treated with iodination conditions such as iodine and bis(trifluoroacetoxy)iodobenzene in a solvent such as dichloromethane, chloroform or carbontetrachloride. Alternatively, iodination may be conducted under acidic conditions such as iodine in a mixture of nitric acid and sulfuric acid. The resulting iodide L-2 may be converted to sulfonamide L-3 in a manner analogous to the procedure set forth in Schemes A and E.

In step o-1, the compound of formula L-3 is coupled to a suitable aryl boronic acid in a manner analogous to the procedure set forth in Scheme J, step n-2. Alternatively, compounds of formula L-3 may be treated with, for example, bis(pinacolato)diboron, a suitable catalyst such as PdCl₂(dppf), a base such as potassium acetate, while heating to 80 to 140° C. in a suitable solvent such as DMF or DMSO to afford the corresponding boronate. Subsequent treatment with standard palladium cross-coupling conditions with a suitable aryl bromide, iodide, or triflate can afford the desired product L-4.

In step o-2, the BOG group of compound L-4 can be cleaved using known methods to afford the amine product B-1. For example, a strong acid such as HCl, H₂SO₄, or TFA may be added to the compound L-4 in a suitable solvent such as ether or dioxane, or alcohols such as methanol or ethanol.

Scheme M depicts a method for the synthesis of hydroxytetrahydropyran compounds of formula I.

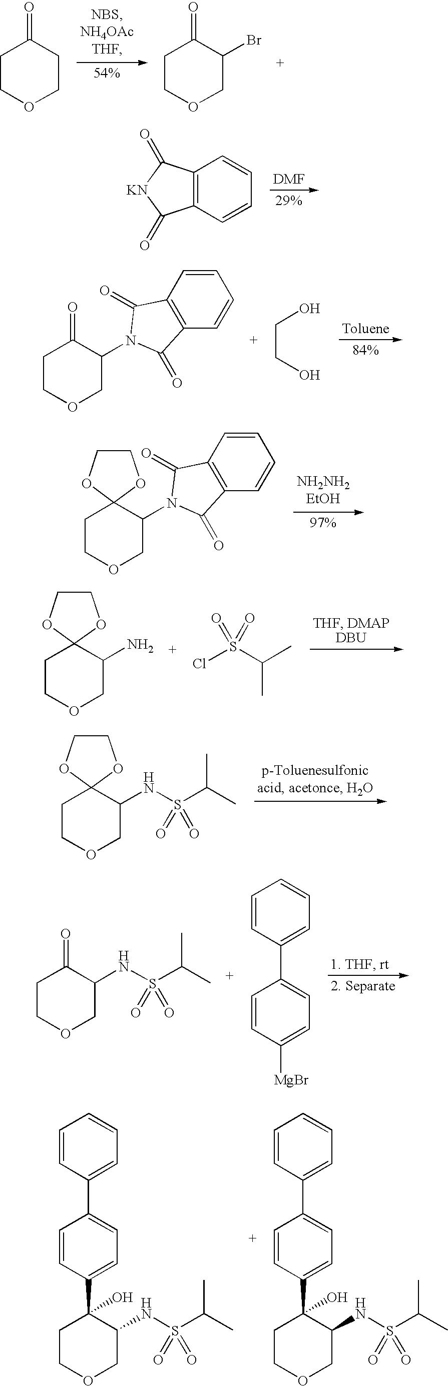

### Working Examples

The following illustrate the synthesis of various compounds of the present invention. Additional compounds within the scope of this invention may be prepared using the methods illustrated in these Examples, either alone or in combination with techniques generally known in the art.

Otherwise specified, the stereochemistry of the compounds in the examples and schemes below is intended to denote relative stereochemistry, rather than absolute stereochemistry.

### Preparation of 1-benzyl-4-trifluoromethanesulfonyloxy-1,2,5,6-tetrahydro-pyridine-3-carboxylic acid ethyl ester

A solution of 15% sodium carbonate (6L) was prepared and to this was added ethyl N-benzyl-3-oxo-piperidine carboxylate hydrochloride (1800 g, 6.06 mol). The slurry was allowed to stir for one hour at which time most of the solids had dissolved. To this was added MTBE (6L). The organic layer was removed and the aqueous layer was extracted twice more with MTBE (2 L each extraction). The combined organic layers were dried over sodium sulfate, filtered, and the solvent was removed by rotary evaporation giving an orange oil (1422 g, 90%). The oil was used without any further purification.

To a room temperature suspension of sodium hydride (120 g, 3.0 mol) in diethyl ether (9 L) was added the free ethyl N-benzyl-3-oxo-piperidine carboxylate (711 g, 2.72 mol) as a solution in diethyl ether (1L). Once the addition was complete the reaction mixture was allowed to stir at room temperature for one hour. Trifluoromethanesulfonic anhydride (460 mL, 2.72 mol) was then added carefully and the reaction mixture was allowed to stir overnight. The reaction was quenched with saturated ammonium chloride and extracted with ethyl acetate. The combined organic layers were dried over sodium sulfate, filtered, and the solvent was removed by rotary evaporation giving 1-benzyl-4-trifluoromethanesulfonyloxy-1,2,5,6-tetrahydro-pyridine-3-carboxylic acid ethyl ester as an orange oil (940 g, 88%). The crude product was used without any further purification.

### Preparation of 1-benzyl-4-biphenyl-4-yl-1,2,5,6-tetrahydro-pyridine-3-carboxylic acid ethyl ester

1-Benzyl-4-trifluoromethanesulfonyloxy-1,2,5,6-tetrahydro-pyridine-3-carboxylic acid ethyl ester (511 g, 1.3 mol), potassium carbonate (431 g, 3.12 mol) and 4-biphenyl boronic acid (175 g, 1.43 mol) were taken up in tetrahydrofuran (6.5 L). The room temperature mixture was purged with nitrogen for 30 minutes at which time tetrakis(triphenylphosphine)palladium(0) (30 g, 0.026 mol) was added and the reaction mixture was heated to 60° C. overnight. The reaction mixture was cooled to room temperature, filtered through a short bed of celite and the solvent was removed by rotary evaporation. The crude oil was taken up in ethyl acetate, washed successively with 10% sodium bicarbonate and water, dried over sodium sulfate, filtered, and the solvent was removed by rotary evaporation giving a black oil. The oil was purified via column chromatography (eluting with 4:1 heptanes:ethyl acetate to 1:1 heptanes:ethyl acetate) to afford the product as a yellow oil (330 g, 80%).

### Preparation of cis-4-biphenyl-4-yl-piperidine-3-carboxylic acid ethyl ester

A suspension of 1-benzyl-4-biphenyl-4-yl-1,2,5,6-tetrahydro-pyridine-3-carboxylic acid ethyl ester (330 g, 1.03 mol) 10% Pd/C (140 g, 50% by wt. water), and acetic acid (65 g) in ethanol (5 L) was heated to 60° C. and charged with 50 psi of hydrogen overnight. The reaction mixture was then cooled to room temperature and filtered though celite (rinsing with ethanol). The solvent was removed by rotary evaporation to afford the product as a reddish semi-solid (261 g, 86%) that was used without further purification.

### Preparation of cis-4-biphenyl-4-yl-piperidine-1,3-dicarboxylic acid 1-tert-butyl ester

A solution of cis-4-biphenyl-4-yl-piperidine-3-carboxylic acid ethyl ester (107 g, 0.46 mol) was taken up in 3M HCl (2.3 L) and heated to reflux overnight. The reaction mixture was cooled to room temperature and then basified to pH ˜10 with solid potassium hydroxide. The material was used without any further purification.

To the now basic solution (assuming 0.46 mol) was added di-tert-butyl dicarbonate (106 g, 0.483 mol) followed by the addition of tetrahydrofuran (560 mL) to help with solubility. The reaction was allowed to stir overnight at room temperature. The solution was then carefully acidified to pH ˜2 with 6M HCl. The aqueous layer was extracted with ethyl acetate. The combined organic layers were dried over sodium sulfate, filtered, and the solvent was removed by rotary evaporation to give an off-white solid that was triturated with ethyl acetate to give the product as a white solid (62 g, 44% overall for the last two steps).

### Preparation of cis-4-biphenyl-4-yl-piperidine-1,3-dicarboxylic acid 1-tert-butyl ester 3-ethyl ester

To a room temperature solution of cis-4-biphenyl-4-yl-piperidine-3-carboxylic acid ethyl ester (130 g, 0.44 mol) in tetrahydrofuran (1.5 L) was added potassium carbonate (152 g, 1.1 mol) followed by di-tert-butyl dicarbonate (100 g, 0.462 mol). The reaction mixture was allowed to stir overnight at room temperature. The solids were removed by suction filtration (rinsing with additional THF). The solvent was removed by rotary evaporation and the crude red oil was used without further purification.

### Preparation of trans-4-biphenyl-4-yl-piperidine-1,3-dicarboxylic acid 1-tert-butyl ester 3-ethyl ester

Crude cis-4-biphenyl-4-yl-piperidine-1,3-dicarboxylic acid 1-tert-butyl ester 3-ethyl ester (assuming 0.44 mol) was taken up in ethanol (1.12 L). Sodium ethoxide (35 g, 0.47 mol) was added. The reaction mixture was heated at reflux for 4 hours (reaction was monitored by ¹H NMR). The reaction mixture was then cooled to room temperature and the solvent was removed by rotary evaporation. The epimerized material was used without further purification.

### Preparation of trans-4-biphenyl-4-yl-piperidine-1,3-dicarboxylic acid 1-tert-butyl ester

To a solution of crude trans-4-biphenyl-4-yl-piperidine-1,3-dicarboxylic acid 1-tert-butyl ester 3-ethyl ester (0.44 mol) in methanol (570 mL) and water (950 mL) was added potassium hydroxide (51 g, 0.90 mol). The reaction was heated to reflux overnight. Once cooled to room temperature the reaction mixture was acidified to pH ˜2 with 6M HCl. The aqueous layer was extracted with ethyl acetate. The combined organic layers were dried over sodium sulfate, filtered, and the solvent was removed by rotary evaporation giving an off-white solid that was triturated with ethyl acetate to give the product a white solid (75.6 g, 56% overall for the three steps). 1H NMR (300 MHz, CDCl3): d=7.55-7.20 (m, 9H), 4.42 (m, 1H), 4.23 (m, 1H), 2.97-2.74 (m, 4H), 1.83 (m, 1H), 1.68 (m, 1H), 1.46 (s, 9H); MS(ES) m/z 404.3 (M+Na); Anal calcd for C₂₃H₂₇NO₄: C, 72.42; H, 7.13; N, 3.67. found C, 72.35; H, 7.26; N, 3.63.

### Preparation of trans-3-amino-4-biphenyl-4-yl-piperidine-1-carboxylic acid tert-butyl ester

To a solution of trans-4-biphenyl-4-yl-piperidine-1,3-dicarboxylic acid 1-tert-butyl ester (5 g, 13.1 mmol) and toluene (50 mL) at 80° C. was added DPPA and triethylamine. The solution was stirred at 80° C. for 3.5 hours. With disappearance of starting material, 2M NaOH (50 mL) and THF (100 mL) were added and stirred vigorously at room temperature overnight. The solution was partitioned between 250 mL of EtOAc and 100 mL 1M NaOH. The organic layer was washed 3×75 mL EtOAc. The organic layer was dried with sodium sulfate, filtered, and concentrated, and the crude material (4.61 g, 99.8%) was carried through to the next step. ¹H-NMR (400 MHz, CD₃OD) 7.592-7.572 (m), 7.421-7.403 (m), 7.384-7.305 (m), 7.300-7.264 (m), 7.248-7.203 (m), 7.118-7.013 (m), 4.335-4.296 (d), 4.179-4.147 (d), 2.932-2.905 (m), 2.895-2.823 (m), 2.576-2.468 (m), 2.459-2.432 (m), 1.797-1.639 (m), 1.481 (s); LC/MS purity=63.19%, ELSD/MS purity=91.67%, calculated for C₂₂H₂₈N₂O₂ 352.5, M⁺ found to be 353.3.

### Preparation of trans-4-biphenyl-4-yl-3-(propane-2-sulfonylamino)-piperidine-1-carboxylic acid tert-butyl ester

To a solution of trans-3-amino-4-biphenyl-4-yl-piperidine-1-carboxylic acid tert-butyl ester (4.61 g, 13.1 mmol) in DOCM (35 mL) was added DBU (4.8 mL, 26.1 mmol). The solution was cooled to 0° C. and propane-2-sulfonyl chloride (3.6 mL, 26.1 mmol) was added dropwise. The resulting solution was allowed to warm to room temperature and stir overnight. The reaction was quenched with ice and partitioned between 2M NaOH (50 mL) and EtOAc (50 mL). The aqueous layer was extracted with 2×40 mL EtOAc. The organic layer was dried with sodium sulfate, filtered, concentrated, and the crude material was purified by chromatography using silica gel (60:40 heptane/EtOAc) to yield the product (2.17 g, 36%) as a solid: ¹H-NMR (400 MHz, CDCl₃) 7.575-7.534 (m), 7.435-7.366 (m), 7.353-7.330 (m), 7.304-283 (m), 7.249 (s), 4.692-4.216 (d/m), 3.927-3.909 (d), 3.420 (s), 2.782-2.752 (t) 2.722-2.609 (m), 2.523-2.456 (m), 1.871-1.810 (m), 1.582 (s), 1.491 (S), 1.131-1.113 (d); LC/MS purity=100%, ELSD/MS purity=100%, calculated for C₂₅H₃₄N₂O₄S 458.6, M⁺ found to be 459.2.

### Example 1

### Preparation of propane-2-sulfonic acid (trans-4-biphenyl-4-yl-piperidin-3-yl)-amide

To a solution of trans-4-biphenyl-4-yl-3-(propane-2-sulfonylamino)-piperidine-1-carboxylic acid tert-butyl ester (2.17 g, 4.7 mmol) in MeOH (20 mL) was added 4M HCl in dioxane (15 mL). The solution stirred at room temperature for 1 hour. The resulting solution was partitioned between 1M NaOH (40 mL) and EtOAc (40 mL). The aqueous layer was extracted with 2×30 mL of EtOAc. The organic layers were combined, dried with sodium sulfate, filtered, concentrated to yield the product (1.74 g) as a glass: ¹H-NMR (400 MHz, CDCl₃) 7.568-7.532 (m), 7.446-7.413 (m), 7.363-7.326 (m), 3.544-3.503 (m), 3.204-3.173 (d), 2.559-2.440 (m), 1.957-1.923 (m), 1.090-1.074 (d), 0.642-0.625 (d); LC/MS purity=97.62%, ELSD/MS purity=100%, calculated for C₂₀H₂₆N₂O₂S 358.51, M⁺ found to be 359.2.

### Example 2

### Preparation of propane-2-sulfonic acid (trans-4-biphenyl-4-yl-1-methyl-piperidin-3-yl)-amide

A solution of propane-2-sulfonic acid (trans-4-biphenyl-4-yl-piperidin-3-yl)-amide (1.74 g, 4.85 mmol), 37% formaldehyde (2.2 ml, 3 eq), NaBH(OAc)₃ (4.1 g, 4 eq), and DCE (113 ml) was stirred under nitrogen for 2 hours. The resulting solution was partitioned between 20 mL of water and 40 mL sat'd NaHCO₃. The aqueous layer was extracted 3×50 ml DCM. The organic layers were combined, dried with sodium sulfate, filtered, concentrated, and the crude material was purified by flash chromatography (90:10 EtOAc/1M NH₃ in MeOH) to yield the product (1.34 g, 71%) as a solid: ¹H-NMR (400 MHz, CD₃OD) 7.578-7.426 (m), 7.422-7.321 (m), 7.311-7.302 (m), 3.583-3.528 (m), 2.934-2.905 (d), 2.496-2.322 (m) 2.108-1.841 (m) 0.998-0.981 (d), 0.590-0.573 (d); LC/MS purity=92.33%, ELSD/MS purity=100%, calculated for C₂₁H₂₈N₂O₂S 372.53, M⁺ found to be 373.2.

### Example 3

### Propane-2-sulfonic acid ((3R,4R)-4-biphenyl-4-yl-1-methyl-piperidin-3-yl)-amide and

### Example 4

### Propane-2-sulfonic acid ((3S,4S)-4-biphenyl-4-yl-1-methyl-piperidin-3-yl/-amide

Racemic propane-2-sulfonic acid (trans-4-biphenyl-4-yl-1-methyl-piperidin-3-yl)-amide (1.11 g) was chromatographed on a chiralpak AD column, eluting with 80:20 Heptane: EtOH to afford the separated enantiomers.

Propane-2-sulfonic acid ((3R,4R)-4-biphenyl-4-yl-1-methyl-piperidin-3-yl)-amide was found to have an optical rotation of +4.63.

Propane-2-sulfonic acid ((3S,4S)-4-biphenyl-4-yl-1-methyl-piperidin-3-yl)-amide was found to have an optical rotation of −4.69.

### Example 5

### Preparation of propane-2-sulfonic acid (trans-4-biphenyl-4-yl-1-ethyl-piperidin-3-yl)-amide

To solution of propane-2-sulfonic acid (trans-4-biphenyl-4-yl-piperidin-3-yl)-amide (19.7 mg, 0.05 mmol) in DCE (700 μL) was added acetaldehyde (9.2 μL, 3 eq) and NaBH(OAc)₃ (47.3 mg, 4 eq). The solution was allowed to stir at room temperature for 1.5 hours. The resulting solution was partitioned between 5 mL sat'd NaHCO₃ and 10 mL EtOAc. The aqueous layer was extracted with 2×10 ml EtOAc. The organic layer was combined, dried with sodium sulfate, filtered, concentrated, and purified by flash chromatography (90:10 EtOAc/1M NH₃ in MeOH) to yield the product (16.6 mg, 78%) as a solid: ¹H-NMR (400 MHz, CDCl₃) 7.582-7.563 (m), 7.431-7.376 (m), 7.330-7.293 (m), 3.586-3.442 (m), 3.398-3.372 (d), 3.052-3.027 (d), 2.644-2.464 (m), 2.343-2.367 (m), 2.080-1.900 (m), 1.171-1.136 (t), 1.001-0.984 (d), 0.593-0.577 (d); LC/MS purity=89.79%, ELSD/MS purity=100%, calculated for C₂₂H₃₀N₂O₂S 386.56, M⁺ found to be 387.2.

### Example 6

### Preparation of propane-2-sulfonic acid (trans-4-biphenyl-4-yl-1-methanesulfonyl-piperidin-3-yl-amide

To a solution of propane-2-sulfonic acid (trans-4-biphenyl-4-yl-piperidin-3-yl)-amide (19.7 mg, 0.05 mmol) in DCM (280 μL) was added triethylamine (9.3 μL, 1.2 eq). This mixture was added dropwise to a solution of ClSO₂CH₃ (5.2 μL, 1.2 eq) in DCM (120 μL). The reaction was stirred under nitrogen for 4.5 hours. The resulting solution was quenched with 500 μL of DCM and the aqueous layer was washed with 5 mL water and 10 mL 1M NaOH. The organic layer was combined, dried, filtered, concentrated, and purified by flash chromatography (95:5 EtOAc/MeOH) to yield the product (10 mg, 45%) as a solid: ¹H-NMR (400 MHz, CDCl₃) 7.602-7.538 (m), 7.465-7.427 (m), 7.382-7.345 (m), 7.321-7.302 (m), 4.404-4.362 (m), 4.404-4.362 (m), 4.004-3.986 (d), 3.624-3.587(d), 3.624-3.587 (m), 2.882 (s), 2.826-2.722 (m), 2.705-2.628 (m), 2.522-2.456(m) 2.067-2.029 (m), 1.139-1.122 (d), 0.659-0.642 (d); LC/MS purity=100%, ELSD/MS purity=100%, calculated for C₂₁H₂₈N₂O₄S₂ 436.6, M⁺ found to be 437.1.

### Example 7

### Preparation of propane-2-sulfonic acid (cis-4-biphenyl-4-yl-1-methyl-piperidin-3-yl)-amide

Using procedures analogous for the conversion of trans-4-biphenyl-4-yl-piperidine-1,3-dicarboxylic acid 1-tert-butyl ester to propane-2-sulfonic acid (trans-4-biphenyl-4-yl-1-methyl-piperidin-3-yl)-amide, cis-4-biphenyl-4-yl-piperidine-1,3-dicarboxylic acid 1-tert-butyl ester was converted to propane-2-sulfonic acid (cis-4-biphenyl-4-yl-1-methyl-piperidin-3-yl)-amide without purification of the intermediates. The product was purified by column chromatography on a silica column with EtOAc/1M ammonia in MeOH. APCl LCMS: Observed mass: 373.19 (M+H). LC/MS/UV purity: 100%. LC/MS/ELSD purity: 100%. 1H NMR (400 MHz, CDCl₃): δ=7.53-7.56 (m, 4H), 7.41-7.45 (t, 2H), 7.31-7.36 (m, 2H), 3.99 (d, 1H), 3.24-3.31 (m, 2H), 2.915 (dd, 1H), 2.715 (d, 1H), 2.53 (s, 3H), 2.39-2.48 (m, 2H), 1.98-2.05 (m, 1H), 1.905 (d, 1H), 0.98 (d, 3H), 0.82 (d, 3H).

General Procedure A

To a solution of carboxylic acid (1 mL of a 0.2 M solution in toluene) was added triethylamine (56 uL, 2 equiv.) and DPPA (86 uL, 2 equiv.). The reactions were stirred at 80° C. for 3.5 hours, then the solvent was removed. THF (2 mL) and 2 M NaOH (1 mL) were added, then the reactions were agitated for an additional 4 hours. Toluene (1 ml) and 2 M NaOH (1 ml) were added, then the organic layer was separated and washed with 2×2 M NaOH. The organic layers were concentrated, dissolved in EtOAc (2 mL), and purified with silica gel SP (eluted withEtOAc/MeOH=80:20) to afford the crude amine intermediates. A solution of DBU (150 uL of 0.2 M solution in DCM, 1.5 equiv.) was then added, followed by a solution of sulfonyl chloride (150 uL of a 0.2 M solution in DCM, 1.5 equiv.). The reactions were stirred at room temperature for 2.5 hours. Additional DBU and sulfonyl chloride were added as needed for the reactions to proceed to completion. After stirring at room temperature for 6.5 hours, the solvents were removed. The crude materials were then dissolved in MeOH (0.5 mL) and treated with 4 M HCl in dioxane (5 equiv. each). The contents were shaken at room temperature for 2 hours. At the end of this time, EtOAc (3 mL) and 1 M NaOH (1.5 mL) were added and the layers were separated. The aqueous layer was extracted with 2× EtOAc, then the organic layers were combined and concentrated. The crude products were dissolved in 1 mL of DMSO and purified by HPLC using a Symmetry 4.6×50 mm, 3.5 um C8 column with a 95/5 water/acetonitrile gradient (0.05% TFA modifier) to afford the examples 8-14.

### Example 8

### N-(trans-4-Biphenyl-4-yl-piperidin-3-yl)-methanesulfonamide

Retention time=1.96 minutes; LCMS/UV purity=100%, LCMS/ELSD purity=100%, calculated for C₁₈H₂₂N₂O2S: 330.14, MH⁺ found to be 331.14.

### Example 9

### Ethanesulfonic acid (trans-4-biphenyl-4-yl-piperidin-3-yl)-amide

Retention time=2.02 minutes; LCMS/UV purity=100%, LCMS/ELSD purity=100%; calculated for C19H24N2O2S: 344.16, MH⁺ found to be 345.16.

### Example 10

### Propane-1-sulfonic acid (trans-4-biphenyl-4-yl-piperidin-3-yl)-amide

Retention time=2.13 minutes; LCMS/UV purity=100%, LCMS/ELSD purity=100%; calulated for C₂₀H₂₆N₂O₂S: 358.17, MH⁺ found to be 359.14.

### Example 11

### Propane-1-sulfonic acid(cis4-biphenyl-4-yl-piperidin-3-yl-amide

Retention time=2.07 minutes; LCMS/UV purity=83.4%, LCMS/ELSD purity=100%; calculated for C₁₉H₂₄N₂O₂S: 344.16, MH⁺ found to be 345.16.

### Example 12

### Propane-1-sulfonic acid(cis-4-biphenyl-4-yl-piperidin-3-yl)-amide

Retention time=2.18 minutes; LCMS/UV purity=100%, LCMS/ELSD purity=100%, calculated for C₂₀H₂₆N₂O₂S: 358.17, MH⁺ found to be 359.15.

### Example 13

### 2,2,2-Trifluoro-ethanesulfonic acid(trans-4-biphenyl-4-yl-piperidin-3-yl)-amide

Retention time=2.22 minutes; LCMS/UV purity=90.5%, LCMS/ELSD purity=100%; calculated for C₁₉H₂₁F₃N₂O₂S: 398.13, MH⁺ found to be 399.06.

### Example 14

### Cyclopropanesulfonic acid(trans-4-biphenyl-4-yl-piperidin-3-yl)-amide

Retention time=2.15 minutes; LCMS/UV purity=100%, LCMS/ELSD purity=100%; calculated for C₂₀H₂₄N₂O₂S: 356.16, MH⁺ found to be 357.14.

### Preparation 1-benzyl-5-trifluoromethanesulfonyloxy-1,2,3,6-tetrahydro-pyridine-4-carboxylic acid ethyl ester

Ethyl N-benzyl-3-oxo-4-piperidine carboxylate HCl (450 g, 1.54 mol) was dissolved in diethyl ether (4 L) and extracted with 10% sodium bicarbonate (2.5 L). The organic layer was dried (sodium sulfate) and the solvent was removed in vacuo to provide 383.6 g of starting ester (96% recovery).

In a 22 L 4-neck flask NaH (64.9 g, 1.62 mol, 60% dispersion) was suspended in anhydrous ether (8 L). The ester (383.6 g, 1.48 mol) was dissolved in ether (800 mL) and added dropwise over a period of 2 hours. After stirring at room temperature for 1 hour, trifluoromethanesulfonic anhydride (248 mL, 1.48 mol) was carefully added and the reaction mixture was stirred overnight. The reaction was quenched with saturated ammonium chloride (2 L) and partitioned with ethyl ether (2×1 L). The combined organic layers were dried (Na₂SO₄), concentrated in vacuo and the product (574.4 g, 99% yield) was used in the next step without purification.

### Preparation of 1-benzyl-5-phenyl-1,2,3,6-tetrahydro-pyridine-4-carboxylic acid ethyl ester

1-Benzyl-5-trifluoromethanesulfonyloxy-1,2,3,6-tetrahydro-pyridine-4-carboxylic acid-ethyl ester (574.4 g, 1.46 mol), K₂CO₃ (483 g, 3.5 mol) and phenyl boronic acid (196 g, 1.61 mol) were suspended in THF (7 L). The solution was degassed with N₂ for 20 minutes. Palladium tetrakistriphenylphyosphine (33.7 g, 0.03 mol) was added and the reaction was heated overnight at 65° C. The cooled reaction was filtered through celite and the solvent removed in vacuo. The crude oil was dissolved in EtOAc (2.5 L) and washed with 10% NaHCO₃ (2L) and water (2 L), dried (Na₂SO₄) and the solvent removed in vacuo to provide the crude product. This material was purified by column chromatography to provide the 445 g of the product in 95% yield.

### Preparation of cis-3-phenyl-piperidine-4-carboxylic acid ethyl ester

1-Benzyl-5-phenyl-1,2,3,6-tetrahydro-pyridine-4-carboxylic acid ethyl ester (445 g, 1.38 mol) was hydrogenated at 40° C. in ethanol (9 L) and acetic acid (356 mL) at 50 psi with 10% Pd/C (90 g) overnight. The catalyst was filtered through celite and the solvent was removed in vacuo. The residue was taken up in ethyl acetate and treated with saturated aqueous bicarbonate, the solvents were then removed in vacuo to give the pure product as a reddish oil (90%).

### Preparation of cis-3-phenyl-piperidine-1,4-dicarboxylic acid 1-tert-butyl ester 4-ethyl ester

To a room temperature solution of cis-3-phenyl-piperidine-4-carboxylic acid ethyl ester (345 g, 1.21 mol) in THF (9.5 L) and water (950 mL) and was added K₂CO₃ (335 g, 2.42 mol) followed by di-tert-butyl dicarbonate (280 g, 1.27 mol). The reaction mixture was stirred overnight. Solvents were removed by rotary evaporation and the residue was taken up in ethyl acetate and washed with water. The solvents were dried over Na₂SO₄ and then removed by rotary evaporation to give the product as a yellowish oil (90%).

### Preparation of trans-3-phenyl-piperidine-1,4-dicarboxylic acid 1-tert-butyl ester

To a room temperature solution of cis-3-phenyl-piperidine-1,4-dicarboxylic acid 1-tert-butyl ester 4-ethyl ester (236 g, 0.71 mol) in ethanol (1.8 L) was added NaOEt (50.7 g, 0.745 mol). The reaction mixture was then heated to reflux for 4 hours. The reaction mixture was cooled to room temperature and the solvent was removed by rotary evaporation. The residue was then poured over saturated aqueous NH₄Cl and extracted with ethyl acetate. The solvent was again removed by rotary evaporation to give trans-3-phenyl-piperidine-1,4-dicarboxylic acid 1-tert-butyl ester 4-ethyl ester as an oil (100%).

To a room temperature solution of trans-3-phenyl-piperidine-1,4-dicarboxylic acid 1-tert-butyl ester 4-ethyl ester (220 g, 0.66 mol) in methanol (3.0 L) ethanol (200 mL) and water (2.2 L) was added KOH (56 g, 1.0 mol) the reaction was heated to reflux overnight. The reaction mixture was cooled to room temperature and the solvents were removed by rotary evaporation. The remaining aqueous layer was acidified with HCl and then extracted with ethyl acetate. The solvent was removed to give the product as an off white solid (76%). The solid was deemed 95.1 pure by LCMS and can be recrystallized from heptane. MS (APCI-ES) m/z 305.0; found 328.2 (M+23). 1H NMR (CDCl3) d=7.34-7.18 (m, 5H), 4.31-4.08 (m, 2H), 2.90 (m, 1H), 2.72 (m, 3H), 1.95 (m, 1H), 1.70 (ml 1H), 1.42 (s, 9H). Anal calcd for C₁₇H₂₃NO₄: C 66.86, H 7.59, N 4.59; found C 66.24, H 7.44 N 4.65.

### Preparation of cis-3-phenyl-piperidine-1,4-dicarboxylic acid 1-tert-butyl ester

To a stirred suspension of 1-benzyl-5-phenyl-1,2,3,6-tetrahydro-pyridine-4-carboxylic acid ethyl ester (205.4 g, 0.517 mol) in MeOH (300 mL) was added a solution of KOH in water (1.2 L, 2.5 M). The reaction mixture was heated at reflux until the LCMS showed the consumption of the starting material (about 48 hours). The reaction mixture was concentrated in vacuo until an oil formed. The solution was acidified to pH 1-2 by concentrated HCl and the resulting solid was collected by vacuum filtration. The acid was used in the next step without further purification.

1-Benzyl-5-phenyl-1,2,3,6-tetrahydro-pyridine-4-carboxylic acid (0.517 mol) was suspended in a 1:1 mixture of THF and water (2.6 L total) in the reaction vessel. To this suspension was added PtO2 (19 g, 10% by weight loading). The reaction vessel was charged with hydrogen gas (50 psi) and the reaction was stirred overnight at 40° C. The reaction mixture was cooled to room temperature and then filtered through a pad of celite (rinsing with 1:1 THF/water) under nitrogen. The filtrate containing cis-3-phenyl-piperidine-4-carboxylic acid (approx. 6 L) was used in the next step without further purification.

To the filtrate containing cis-3-phenyl-piperidine-4-carboxylic acid (0.517 mol) was added potassium carbonate (142.7 g, 1.034 mol) and (BOC)2O (118.3 g, 0.543 mol). The reaction was stirred until the LCMS indicated the consumption of the starting material (4-6 h). The reaction mixture was brought to pH 1-2 by concentrated HCl (˜86 mL) and extracted with dichloromethane (4 L). The organic phase was filtered to provide the product (46 g). The organic phase was concentrated in vacuo and filtered to provide additional product (64 g). The recovered material was combined and triturated in heptane/EtOAc to provide cis-3-phenyl-piperidine-1,4-dicarboxylic acid 1-tert-butyl ester (107 g, 54.4%) as a white solid. MS (APCI-ES) m/z 305.0; found 328.2 (M+23). NMR (CDCl3) d=7.20 (m, 5H), 4.05 (br, 1H), 3.70 (br, 2H), 3.47 (br, 1H), 3.12 (m, 1H), 2.93 (m, 1H), 1.93 (m, 1H), 1.82 (m, 1H), 1.40 (br s, 9H). Anal calcd for C₁₇H₂₃NO₄: C 66.86, H 7.59, N 4.59; found C 65.81, H 7.50, N 4.77.

### Preparation of 4-hydroxy-butyric acid ethyl ester

A 500 mL round bottom flask equipped with a magnetic stir bar was charged with 125 mL of absolute ethanol, 5.0 g (58.1 mmols) of gamma-butyrolactone and 25 g of activated Amberlyst 15 resin (resin was activated by washing 2× with ˜250 ml of 2.0 N HCl, 1× with ˜250 mL of THF, 1× with 250 mL of ethanol, and dried under vacuum until light grey and fluid.) The reaction was capped and stirred gently for 96 hours at room temperature. The resin was removed from reaction by filtration, the resin was washed (ethanol) and the filtrate was treated with solid K₂CO₃ until neutral by pH paper. The filtrate was concentrated at reduced pressure (water bath 20° C.) to give a white solid slurry. The slurry was dissolved in ether, filtered through a plug of celite, solids were washed 2× with ether, and the filtrate was concentrated at reduced pressure (water bath 20° C.) to give the desired 4-hydroxy-butyric acid ethyl ester (5.85 g, 76.2%, purity ˜70%) or (4.18 g, 54%) as clear oil in a 5:2 ratio with the starting material. 1H NMR (400 MHz, CHLOROFORM-d) δ ppm 1.15-1.23 (m, 4H), 1.78-1.86 (m, 2H), 2.37 (t, 2H). 3.59-3.67 (m, 3H), 4.08 (q, 2H).

### Preparation of 4-ethoxycarbonylmethoxy-butyric acid ethyl ester

A 1.0 L round bottom flask equipped with a magnetic stir bar, addition funnel and nitrogen blanket was charged with 4-hydroxy-butyric acid ethyl ester (19.08 g, 70%, 100 mmols), rhodium (II) acetate (445 mg, 1.01 mmols) and methylene chloride (400 mL). Ethyl diazoacetate (18.1 mL, 152 mmols) in methylene chloride (100 mL) was added dropwise via an addition funnel over a period of 1.5 hrs and the reaction was stirred at room temperature for 72 hrs. The reaction mixture was filtered through a plug of silica gel, the plug was washed with methylene chloride, and filtrate was concentrated to a light yellow oil. The crude oil was purified by fractional distillation at reduced pressure. The fraction boiling between 70-80° C. at ˜1 torr was collected to give the product (19.20 g, 87%) as clear oil. 1H NMR (400 MHz, CHLOROFORM-d) δ ppm 1.22-1.29 (m, 6H), 1.89-1.97 (m, 2H), 2.43 (t, 2H), 3.56 (t, 2H), 4.04 (s, 2H), 4.12 (q, 2H), 4.20 (q, 2H)

### Preparation of 5-hydroxy-3,6-dihydro-2H-pyran-4-carboxylic acid ethyl ester

A 1.0 L round bottom flask equipped with a magnetic stir bar and nitrogen blanket was charged with 4-ethoxycarbonylmethoxy-butyric acid ethyl ester (15.01 g, 68.77 mmols) and toluene (300 mL). A 1.0 M solution of potassium tert-butoxide solution in THF (82.0 mL, 82 mmols) was added to the reaction via syringe over the course of 10 minutes at room temperature and stirred for 18 hours. The reaction was poured onto 300 mL of 1.0 N HCl, the organic phase was separated and the aqueous phase was extracted with ether. The combined organic phases were washed with water, saturated brine, dried (Na₂SO₄), filtered and concentrated to give 11.21 g of an orange oil. The crude oil was purified by flash chromatography on a 330 g RediSep column with 1:19 ethyl acetate:heptane. The combined product fractions were concentrated to give the product as light yellow oil (7.21 g, 60.9%). 1H NMR (400 MHz, CHLOROFORM-d) δ ppm 1.32 (t, 3H), 2.33-2.37 (m, 2H), 3.79 (t, 2H), 4.12-4.15 (m, 2H), 4.25 (q, 2H), 11.85 (s, 1H)

### Preparation of 5-trifluoromethanesulfonyloxy-3,6-dihydro-2H-pyran-4-carboxylic acid ethyl ester

A 125 mL 3-neck round bottom flask equipped with a magnetic stir bar, septum and nitrogen blanket was charged with sodium hydride (275 mgs, 6.88 mmols, 60% suspension in mineral oil.) The sodium hydride was washed heptane (2×20 mL) and the flask was charged with anhydrous diethyl ether (30 mL.). To the stirring suspension was added dropwise a solution of 5-hydroxy-3,6-dihydro-2H-pyran-4-carboxylic acid ethyl ester (1.03 g, 5.98 mmols) in anhydrous ether (5 mL) and allowed to stirred at room temperature for 1 hour. To the reaction was added trifluoromethanesulfonic anhydride (1.01 mL, 5.98 mmols) and the reaction was stirred at room temperature overnight. The reaction was quenched with saturated aqueous ammonium chloride solution, the organic layer was separated, washed with saturated brine, dried (MgSO₄), filtered and concentrated under reduced pressure to give the product (1.63 g, 89%) as yellow oil to be used without further purification. 1H NMR (400 MHz, CHLOROFORM-d) δ ppm 1.35 (t, 3H), 2.61-2.66 (m, 2H), 3.82 (t, 2H), 4.19 (t, 2H), 4.32 (q, 2H)

### Preparation of 5-biphenyl-4-yl-3,6-dihydro-2H-pyran-4-carboxylic acid ethyl ester

To a 35 mL round bottom flask equipped with a magnetic stir bar, condenser and vacuum/nitrogen inlet was charged 5-trifluoromethanesulfonyloxy-3,6-dihydro-2H-pyran-4-carboxylic acid ethyl ester (792 mgs, 2.60 mmols), K₂CO₃ (870 mgs, 6.29 mmols) and 4-diphenylboronic acid (566 mgs, 2.86 mmols) and anhydrous THF (13 mL). The reaction was purged five times with alternating nitrogen and vacuum while stirring vigorously. To the reaction was added Pd(PPh₃)₄ (91.1 mgs, 0.078 mmols) and the reaction was heated at 65° C. under a blanket of nitrogen for 24 hours. The reaction was concentrated to dryness and partitioned between ethyl acetate and water. The aqueous layer was extracted with ethyl acetate (2×) and the combined organic layers were washed with water (2×), saturated brine, dried (MgSO₄), filtered and concentrated to give a dark yellow oil that was pre-loaded on silica gel and purified by column chromatography on a Biotage 40M column (9:46 ethyl acetate:heptane). The combined product fractions were concentrated to give a clear oil that solidified to afford the product (711 mgs, 88.6%) as white solid. 1H NMR (300 MHz, CHLOROFORM-d) δ ppm 0.91 (t, 3H), 2.55-2.61 (m, 2H), 3.91-4.00 (m, 4H), 4.35 (t, 2H), 7.23 (d, 2H), 7.33-7.40 (m, 1H), 7.45 (t, 2H), 7,59 (t, 4H)

### Preparation of cis-3-biphenyl-4-yl-tetrahydro-pyran-4-carboxylic acid ethyl ester

A 250 mL Parr bottle was charged with 5-biphenyl4-yl-3,6-dihydro-2H-pyran-4-carboxylic acid ethyl ester (711 mgs, 2.31 mmols), 10% Pd on carbon (70 mgs) and absolute ethanol (30 mL.) Reaction was shaken on a Parr shaker at 50 psi of hydrogen overnight at room temperature. TLC (1:2 ethyl acetate:heptan-e) of the reaction showed presence of starting material. To the reaction was added an additional 70 mgs of 10% Pd on carbon and the reaction was shaken on a Parr shaker at 70 psi of hydrogen for an additional 6 hours. The reaction was filtered through a pad a celite and the filtrate was concentrated to give the product (705 mgs, 98%) as a light brown oil to be used crude without further purification. 1H NMR (400 MHz, CHLOROFORM-d) δ ppm 1.10 (t, 3H), 1.77-1.85 (m, 1H), 2.07-2.17 (m, 1H), 3.01-3.07 (m, 1H), 3.29 (q: 1H), 3.62-3.69 (m, 1H), 3.90-3.95 (m, 1H), 3.97-4.02 (m, 2H), 4.14-4.21 (m, 1H), 4.33 (dd, 1H), 7.32-7.36 (m, 1H), 7.44 (t, 2 H), 7.46-7.53 (m, 4H), 7.57-7.61 (m, 2H)

### Preparation of trans-3-biphenyl-4-yl-tetrahydro-pyran-4-carboxylic acid ethyl este

To a 50 mL round bottom flask equipped with a magnetic stir bar, reflux condenser and nitrogen blanket was charged cis-3-biphenyl-4-yl-tetrahydro-pyran-4-carboxylic acid ethyl ester (550 mgs, 1.77 mmols) and absolute ethanol (7 mL). To the reaction was added 21% wt/wt NaOEt/EtOH solution (0.695 mL, 1.86 mmols) and reaction was heated at reflux overnight. The reaction was cooled to room temperature, concentrated and quenched with sat. NH4Cl solution. The aqueous layer was extracted 2× with methylene chloride and the combined organic layers were dried and concentrated to give a light-brown oil that solidified to afford the crude product (434.4 mgs, 79%). LC/MS analysis showed it to contain ester hydrolysis byproduct and it was use without further purification. 1H NMR (400 MHz, CHLOROFORM-d) δ ppm 1.03 (t, 2H), 1.94-2.05 (m, 2H), 2.86-2.99 (m, 1H), 3.14-3.21 (m, 1H), 3.42 (q, 1H), 3.51-3.59 (m, 1H), 3.93-4.03 (m, 3H), 4.10-4.15 (m, 1H), 7.28-7.36 (m, 3H), 7.40-7.46 (m, 2H), 7.51-7.59 (m, 4H).

### Preparation trans-3-biphenyl-4-yl-tetrahydro-pyran-4-carboxylic acid

To a 35 mL round bottom flask equipped to magnetic stir bar and reflux condenser was charged trans-3-biphenyl-4-yl-tetrahydro-pyran-4-carboxylic acid ethyl ester (430 mgs, 1.39 mmols), KOH (110 mgs, 1.96 mmols), methanol (6.5 mL), ethanol (0.50 mL), and water (5 mL). The reaction was heated at reflux for 4 hours, removed from heat and stirred at room temperature for 72 hours. The reaction was concentrated to a slurry, diluted with water and washed 2× with ether (discarded). The aqueous layer was made pH=1 with conc. HCl, and extracted 3× with methylene chloride. The combined methylene chloride extractions were dried, filtered and concentrated to afford the product (375 mgs, 95%) as a light brown foam to be used without further purification. 1H NMR (400 MHz, CHLOROFORM-d) δ ppm 1.91-2.04 (m, 2H), 2.93 (td, 1H), 3.16 (td, 1H), 3.38 (t, 1H), 3.54 (td, 1H), 3.98 (dd, 1H), 4.09-4.14 (m, 1H), 7.29 (d, 2H), 7.32-7.36 (m, 1H), 7.42 (t, 2H), 7.52 (d, 2H), 7.55 (d, 2H).

### Preparation of trans-3-biphenyl-4-yl-tetrahydro-pyran-4-ylamine

To a 35 mL round bottom flask equipped with magnetic stir bar, condenser, blanket of nitrogen was charged trans-3-biphenyl-4-yl-tetrahydro-pyran-4-carboxylic acid (365 mgs, 1.29 mmols), (i-Pr)₂NEt (0.419 mL, 1.94 mmols) and toluene (7 mL). DPPA (0.338 mL, 1.94 mmols) was added to the reaction and reaction was heated in an oil bath at 85° C. for 2.5 hours. After cooling to room temperature, THF (7.0 mL) and 2.0 N NaOH (3.5 mL) were added and the reaction was stirred vigorously at room temperature for 3.5 hours. The organic phase was separated and the aqueous layer was extracted with ethyl acetate. The combined organic phases were washed with water (2×), saturated brine, dried (MgSO4), filtered and concentrated to give a yellow oil. The resulting oil was purified by column chromatography on a Biotage 40S column with 1:19 1.0 N NH3 in MeOH:methylene chloride. The combined product fractions were concentrated to give a light yellow oil that solidified upon sitting to afford the product (245 mgs, 75%). 1H NMR (400 MHz, CHLOROFORM-d) δ ppm 1.62-1.73 (m, 1H), 1.96-2.03 (m, 1H), 2.65-2.73 (m, 1H), 3.14-3.22 (m, 1H), 3.40 (t, 1H), 3.52-3.60 (m, 1H), 3.94 (dd: 1H), 4.08 (dd, 1H), 7.30 (d, 2H), 7.33-7.38 (m, 1H), 7.45 (t, 2H), 7.55-7.60 (m, 4H). Mass Spectrum (ES-MS): M+1=254.1.

### Example 15

### Propane-2-sulfonic acid(trans-3-biphenyl-4-yl-tetrahydro-pyran-4-yl)-amide

To an 8 mL vial equipped with septum cap and magnetic stir bar was charged trans-3-biphenyl-4-yl-tetrahydro-pyran-4-ylamine (27.1 mgs, 107 μmols), DBU (32 μL, 210 μmols) and methylene chloride (0.50 mL). The reaction was cooled to 0° C. in an ice/water bath and isopropylsulfonyl chloride was added to the reaction. The reaction was stirred at 0° C. for 5 minutes and then allowed to warm to room temperature and stirred for an additional 30 minutes. The reaction was washed with saturated bicarbonate solution, the organic layer was dried and loaded onto a methanol conditioned (1×4 mL) Waters MCX SPE column. The organics were collected, the column was washed with methylene chloride (3 mL) and the combined organics were concentrated to give 32 mgs of a white foam that was purified by column chromatography on a Biotage 12M column in 1:2 ethyl acetate:heptane. The combined product fractions were concentrated to afford the product as a white solid (19 mgs, 49%). 1H NMR (400 MHz, METHANOL-d₄) δ ppm 0.69 (d, 3H), 1.04 (d, 3H), 1.72-1.84 (m, 1H), 2.12-2.19 (m, 1H), 2.39-2.48 (m, 1H), 2.75-2.83 (m, 1H), 3.55-3.72 (m, 3H), 3.91 (dd, 1H), 4.02 (dd, 1H), 7.30-7.35 (m, 1H), 7.39-7.45 (m, 4H), 7.58 (dd, 4H). Mass Spectrum (ES-MS) M+1=360.2.

### Example 16

### Ethanesulfonic acid(trans-3-biphenyl-4-yl-tetrahydro-pyran-4-yl)-amide

To an 3 mL vial equipped with septum cap and magnetic stir bar was charged trans-3-biphenyl-4-yl-tetrahydro-pyran-4-ylamine (26.8 mgs, 106 μmols), (iPr)₂NEt (46.1 μL, 265 μmols) and methylene chloride (0.50 mL). To the reaction was added ethylsulfonyl chloride (20.2 μL, 212 μmols) and the reaction was stirred overnight at room temperature. The reaction was washed with saturated bicarbonate solution, the organic layer was dried and then loaded onto a methanol conditioned (1×4 mL) Waters MCX-SPE column. The organics were collected, the column was washed with methylene chloride (3 mL) and the combined organics were concentrated to give 32 mgs of a white foam that was purified by column chromatography on a Biotage 12M column in 1:2 ethyl acetateheptane. The combined product fractions were concentrated to afford the product as a white solid (23.5 mgs, 64%). 1H NMR (400 MHz, METHANOL-d₄) δ ppm 0.73 (t, 3H), 1.73-1.83 (m, 1H), 2.11-2.18 (m, 1H), 2.33 (dq, 1H), 2.45-2.55 (m, 1H), 2.75-2.82 (m, 1H), 3.55-3.72 (m, 3H), 3.91 (dd, 1H), 4.03 (dd, 1H), 7.31-7.36 (m, 1H), 7.40-7.45 (m, 4H), 7.60 (dd, 4H). Mass Spectrum (ES-MS) M+1=346.2.

### Preparation of cis-3-Biphenyl-4-yl-tetrahydro-pyran-4-carboxylic acid

The title compound was prepared in a manner analogous to the procedure for trans-3-biphenyl-4-yl-tetrahydro-pyran-4-carboxylic acid from trans-3-biphenyl-4-yl-tetrahydro-pyran-4-carboxylic acid ethyl ester using aqueous NaOH. The reaction resulted in a mixture of the cis and trans acid and 28% of the title compound was isolated by column chromatography with 1:1 ethyl acetate:heptane spiked with glacial acetic acid as an off-white solid containing approximately 10% trans isomer to be used without additional purification. 1H NMR (400 MHz, CHLOROFORM-d) δ ppm 1.76-1.85 (m, 1H), 2.05-2.16 (m, 1H), 3.06-3.11 (m, 1H), 3.29 (q, 1H), 3.60-3.67 (m, 1H), 3.91 (dd, 1H), 4.12-4.18 (m, 1H), 4.32 (dd, 1H), 7.30-7.36 (m, 1H), 7.42 (t, 2H), 7.48-7.58 (m, 6H). Mass Spectrum (ES-MS) M−1=281.3.

### Preparation of cis-3-biphenyl-4-yl-tetrahydro-pyran-4-ylamine

The title compound was prepared in a manner analogous to the procedure for trans-3-biphenyl-4-yl-tetrahydro-pyran-4-ylamine from trans-3-biphenyl-4-yl-tetrahydro-pyran-4-carboxylic acid to give the product as a light brown gum to be used without purification. 1H NMR (400 MHz, CHLOROFORM-d) δ ppm 1.72-1.81 (m, 1H), 1.83-1.92 (m, 1H), 3.09 (q, 1H), 3.46-3.51 (m, 1H), 3.60-3.66 (m, 1H), 3.83 (dd, 1H), 3.98-4.05 (m, 1H), 4.24 (dd, 1H), 7.35 (t, 2H), 7.44 (t, 3H), 7.47-7.53 (m, 2H), 7.54-7.61 (m, 5H). Mass Spectrum (ES-MS) M+1=254.3.

### Example 17

### Propane-2-sulfonic acid(cis-3-biphenyl-4-yl-tetrahydro-pyran-4-yl)-amide

The title compound was prepared from cis-3-biphenyl-4-yl-tetrahydro-pyran-4-ylamine in a manner analogous to the procedure described for example 15 to give 40% of the product as a white solid. 1H NMR (400 MHz, METHANOL-d₄) δ ppm 1.02 (d, 3H), 1.15 (d, 3H), 1.84-1.93 (m, 2H), 2.80-2.89 (m, 1H), 3.16-3.22 (m, 1H), 3.72-3.79 (m, 1H), 3.91 (dd, 2H), 3.97-4.03 (m, 1H), 4.17 (dd, 1H), 7.30-7.35 (m, 1H), 7.42 (t, 2H), 7.50-7.54 (m, 2H), 7.57-7.62 (m, 4H). Mass Spectrum (ES-MS) M+1=360.2.

### Preparation of 5-biphenyl-4-yl-3,6-dihydro-2H-pyran-4-carboxylic acid

The title compound was prepared from 5-biphenyl-4-yl-3,6-dihydro-2H-pyran-4-carboxylic acid ethyl ester in a manner analogous to the preparation of trans-3-biphenyl-4-yl-tetrahydro-pyran-4-carboxylic acid to give 16% of the product as a white crystalline solid after preparative HPLC purification on a Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. 1H NMR (400 MHz, CHLOROFORM-d) δ ppm 2.54-2.59 (m, 2H), 3.91 (t, 2H), 4.33 (t, 2H), 7.24 (d, 2H), 7.34-7.39 (m, 1H), 7.44 (t, 2H), 7.58 (t, 4H). Mass Spectrum (ES-MS) M+1=281.2.

### Preparation of trans-4-phenyl-piperidine-1,3-dicarboxylic acid 1-tert-butyl ester

According to the methods described above, ethyl N-benzyl-3-oxo-piperidine carboxylate hydrochloride (300 g, 1.15 mol) and phenylboronic acid (114.7 g, 0.94 mol) were used to prepare the product (40 g) as a white solid. MS m/z 304.2 (M−1). 1H NMR (MeOH-d4) d=7.3-7.1 (m, 5H), 4.34 (m, 1H), 4.19 (m, 1H), 2.94 (m, 3H), 2.67 (m, 1H), 1.78 (m, 1H), 1.62 (m, 1H), 1.49 (2, 9H).

### Preparation of trans-3-phenyl-piperidine-1,4-dicarboxylic acid 1-tert-butyl ester 4-methyl ester

To a 100 mL round bottom flask equipped with magnetic stir bar was charged trans-3-phenyl-piperidine-1,4-dicarboxylic acid 1-tert-butyl ester (1.528 g, 5.004 mmols) and anhydrous methanol (25 mL). The reaction was cooled to 0° C. in an ice bath and a 2.0 M TMS-diazomethane in hexane solution was added until no acid was present in reaction by TLC (approximately 7 mL of TMS-diazomethane solution, 14 mmols). The reaction was stirred at 0° C. for 30 minutes, quenched with 2 drops of glacial acetic acid and concentrated. The crude reaction was purified by column chromatography on a Biotage 40M in 1:4 ethyl acetate:heptane. The combined product fractions were concentrated to give a light yellow oil that solidified to give the product (1.40 g, 87%). 1H NMR (400 MHz, CHLOROFORM-d) δ ppm 1.47 (s, 9H), 1.72-1.83 (m, 1H), 1.98 (dd, 1H), 2.72-2.85 (m, 3H), 2.92-3.00 (m, 1H), 3.47 (s, 3H), 4.14-4.33 (m, 2H), 7.20-7.26 (m, 3H), 7.28-7.33 (m, 2H). Mass Spectrum (ES-MS): M+1=320.2.

### Preparation of trans-3-(4-iodo-phenyl)-piperidine-1,4-dicarboxylic acid 1-tert-butyl ester 4-methyl ester

A 25 mL round bottom flask equipped with magnetic stir bar was charged with trans-3-phenyl-piperidine-1,4-dicarboxylic acid 1-tert-butyl ester 4-methyl ester (1.60 mgs, 0.501 mmols) and methylene chloride (2.5 mL). Iodine (129 mgs, 0.501 mmols) was added and the reaction stirred at room temperature until iodine was in solution (˜5 min). PhI(O₂CCF₃)₂ (245 mgs, 0.55 mmols) was added and the reaction was stirred at room temperature for 15 minutes. The reaction was quenched with 10% aqueous sodium thiosulfate solution, the organic layer was separated and the aqueous layer was extracted extracted 2× with methylene chloride. The combined organics were dried and concentrated to give a yellowish-brown oil that was triturated with hexanes. Insoluble material was removed by filtration and the filtrate was concentrated and purified by column chromatography on a Biotage 40S column with 3:17 ethyl acetate:hexanes. The combined product fractions were concentrated to give the product (126.7 mgs, 56.8%) as a yellow oil containing approximately 20% of the des-iodo material and was used without further purification. 1H NMR (400 MHz, CHLOROFORM-d) δ ppm 1.47 (s, 11H), 1.69-1.83 (m, 1H), 1.98 (dd, 1H), 2.66-2.85 (m, 3H), 2.85-2.98 (m, 1H), 3.49 (s, 3H), 6.98 (d, 2H), 7.63 (d, 2H).

### Preparation of trans-3-(4-iodo-phenyl)-piperidine-1,4-dicarboxylic acid 1-tert-butyl ester

A mixture of trans-3-(4-iodo-phenyl)-piperidine-1,4-dicarboxylic acid 1-tert-butyl ester 4-methyl ester (540 mgs, 1.21 mmols), 1.0 N aqueous NaOH (3.0 mL, 3.0 mmols) and acetone (6 mL) were stirred overnight at room temperature. The reaction was concentrated to dryness and the resulting residue was partitioned between water and ether. The aqueous layer was made pH=14 with 1.0 N NaOH and extracted with ether (discarded). The aqueous layer was made pH=2 with 1.0 N HCl and extracted 3× with ethyl acetate. The combined ethyl acetate extractions were dried (MgSO4), filtered and concentrated to give the product (510 mgs, 97%) as an off-white solid containing approximately 10% des-iodo product to be used without further purification. 1H NMR (400 MHz, DMSO-d₆) δ ppm 0.55 (s, 11H), 0.58-0.67 (m, 1H) 1.08 (dd, 1H), 1.84 (dd, 1H), 1.88-2.00 (m, 2H), 3.13-3.23 (m, 1H), 6.25 (d, 2H), 6.82 (d, 2H), 11.26 (br. s., 1H). Mass spectrum (ES-MS) M−1=430.1.

### Preparation of trans-4-amino-3-(4-iodo-phenyl)-piperidine-1-carboxylic acid tert-butyl ester

The title compound was prepared in a manner analogous to the preparation of trans-3-biphenyl-4-yl-tetrahydro-pyran-4-ylamine to give 92% of the product as a brown oil containing approximately 15% of the des-iodo compound to be used crude without further purification. Mass Spectrum (ES-MS): M+1=403.1.

### Preparation of trans-3-(4-iodo-phenyl)4-(propane-2-sulfonylamino)-piperidine-1-carboxylic acid tert-butyl ester

The title compound was prepared in a manner analogous to the preparation of example 15 to give 86% of the product as a light brown foam containing approximately 15% of the des-iodo compound to be used crude without further purification. Mass Spectrum (ES-MS): M+1=403.1.

Crude 1H NMR (400 MHz, METHANOL-d₄) δ ppm 0.72 (d, 2H), 1.07 (d, 2H), 1.46 (s, 12H), 1.51-1.62 (m, 1H), 2.12-2.21 (m, 1H), 2.44-2.61 (m, 2H), 2.86-3.06 (m, 2H), 3.54-3.66 (m, 1H), 4.01-4.10 (m, 1H), 4.14 (d, 1H), 7.13 (d, 2H), 7.70 (d, 1H).

### Preparation of trans-3-biphenyl-4-yl-4-(propane-2-sulfonylamino)-piperidine-1-carboxylic acid tert-butyl ester

An 8 mL vial equipped with stir bar and septum cap was charged with a mixture of trans-3-(4-iodo-phenyl)-4-(propane-2-sulfonylamino)-piperidine-1-carboxylic acid tert-butyl ester (120 mgs, 0.236 mmols), phenylboronic acid (46.1 mgs, 0.378 mmols), Na₂CO₃ (27 mgs, 0.45 mmols), ethanol (0.45. mLs), water (0.45 mLs) and toluene (0.90 mL). The reaction was purged 3× with alternating vacuum/nitrogen and Pd(PPh₃)₄ (9.8 mgs, 0.0085 mmols) was added to the reaction. The reaction was again purged 3× with alternating vacuum/nitrogen and heated at 85° C. for 3 hours. The reaction mixture was cooled to room temperature and partitioned between ethyl acetate and water. The aqueous layer-was discarded and the organics were washed with water, saturated brine, dried (MgSO4), filtered and concentrated to give a brown oil that was purified by column chromatography on a Biotage 12M column with 1:4 ethyl acetate:heptane. The combined product fractions were concentrated to the target compound (91 mgs, 84%) as an off-white solid containing a minor impurity of the mono-phenyl compound to be used without further purification. 1H NMR (400 MHz, CHLOROFORM-d) δ ppm 0.73 (d, 3H), 1.12 (d, 3H), 1.48 (s, 9H), 1.58 (dd, 1H), 2.37 (dd, 1H), 2.49 (t, 1H), 2.58 (td, 1H), 2.92 (t, 2H), 3.57-3.66 (m, 1H), 4.19-4.30 (m, 2H), 7.33 (d, 2H), 7.36-7.42 (m, 1H), 7.38 (d, 1H), 7.46 (t, 2H), 7.54-7.62 (m, 4H). Mass Spectrum (ES-MS): M+1=403.2.

### Preparation of trans-3-(4′-cyano-biphenyl-4-yl)-4-(propane-2-sulfonylamino)-piperidine-1-carboxylic acid tert-butyl ester

The title compound was prepared in a manner analogous to the preparation of trans-3-biphenyl-4-yl-4-(propane-2-sulfonylamino)-piperidine-1-carboxylic acid tert-butyl ester to give 46% of the product as an off white solid after HPLC purification on a Waters SunFire C18 ODB Sum 19×100 mm column, gradient: 85% A:15% B to 100% B over 6 minutes with 2 minute hold (A=0.05% TFA in water, B=0.05% TFA in acetonitrile), flow rate: 35 mL/min. Mass Spectrum (ES-MS): M+1=484.2.

### Preparation of trans-3-(2′-cyano-biphenyl-4-yl)-4-(Propane-2-sulfonylamino)-piperidine-1-carboxylic acid tert-butyl ester

The title compound was prepared in a manner analogous to the preparation of trans-3-biphenyl-4-yl-4-(propane-2-sulfonylamino)-piperidine-1-carboxylic acid tert-butyl ester to give 20% of the product after HPLC purification on a Waters SunFire G18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. Mass Spectrum (ES-MS): M+1=484.2.

### Preparation of trans-3-(2′-carbamoyl-biphenyl-4-yl)-4-(propane-2-sulfonylamino)-piperidine-1-carboxylic acid tert-butyl ester

The title compound was prepared as an isolated byproduct during the preparation of trans-3-(2′-cyano-biphenyl-4-yl)-4-(propane-2-sulfonylamino)-piperidine-1-carboxylic acid tert-butyl ester The product was isolated by HPLC purification on a Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. Mass Spectrum (ES-MS): M+1=502.2.

### Preparation of trans-3-(2′-chloro-biphenyl-4-yl)-4-(propane-2-sulfonylamino)-piperidine-1-carboxylic acid tert-butyl ester

The title compound was prepared in a manner analogous to the preparation of trans-3-biphenyl-4-yl-4-(propane-2-sulfonylamino)-piperidine-1-carboxylic acid tert-butyl ester to give 49% of the product after HPLC purification on a Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. Mass Spectrum (ES-MS): M+1=493.1.

### Preparation of trans-3-(2′,5′-dichloro-biphenyl-4-yl)-4-(propane-2-sulfonylamino)-piperidine-1-carboxylic acid tert-butyl ester

The title compound was prepared in a manner analogous to the preparation of trans-3-biphenyl-4-yl-4-(propane-2-sulfonylamino)-piperidine-1-carboxylic acid tert-butyl ester to give 46% of the product after HPLC purification on a Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. Mass Spectrum (ES-MS): M+1=527.1.

### Example 18

### Propane-2-sulfonic acid(trans-3-biphenyl-4-yl-piperidin-4-yl)-amide

A mixture of trans-3-biphenyl-4-yl-4-(propane-2-sulfonylamino)-piperidine-1-carboxylic acid tert-butyl ester (18.3 mgs, 0.0399 mmols), methylene chloride (5 mL) and TFA (1 mL) were stirred overnight at room temperature. The reaction was concentrated to dryness and purified by HPLC on the Shimadzu under the following conditions: Waters SunFire C18 ODB 5 um 19×100 mm column, gradient: 85% A, 15% B to 100% B over 6 minutes with 2 minute hold (A=0.06% TFA in water, B=0.05% TFA in acetonitrile), flow rate: 35 mL/min. The concentrated product fraction was dissolved in methanol and loaded onto a Waters MCX SPE column (conditioned wtih 4 mL of MeOH), the column was washed with 4 mL MeOH and the product was is eluted with 4 mL of 1.0 N NH4/MeOH. The elution fraction was concentrated to dryness to give the product (9.0 mgs, 63%) as a white solid. 1H NMR (400 MHz, METHANOL-d₄) δ ppm 0.66 (d, 3H), 1.02 (d, 3H), 1.59-1.70 (m, 1H), 2.19-2.25 (m, 1H), 2.36-2.44 (m, 1H), 2.62-2.71 (m, 1H), 2.76-2.87 (m, 2H), 3.07-3.15 (m, 2H), 3.53-3.60 (m, 1H), 7.33 (t, 1H), 7.38-7.45 (m, 4H), 7.58 (d, 4H). Mass Spectrum (ES-MS): M+1=359.2.

### Example 19

### Propane-2-sulfonic acid[trans-3-(4′-cyano-biphenyl-4-yl)-piperidin-4-yl]-amide

A mixture of trans-3-(4′-cyano-biphenyl-4-yl)-4-(propane-2-sulfonylamino)-piperidine-1-carboxylic acid tert-butyl ester (18 mgs, 0.037 mmols), methylene chloride (0.75 mL) and TFA (0.25 mL) were stirred for 0.75 hours at room temperature. The reaction was concentrated to dryness, dissolved in methanol, loaded onto a Waters MCX SPE column (conditioned with 4 mL of MeOH), the column was washed with 4 mL MeOH and the product was eluted with 4 mL of 1.0 N NH4/MeOH. The elution fraction was concentrated to dryness to give the product (13.5 mgs, 95%) as a white solid. 1H NMR (400 MHz, METHANOL-d₄) δ ppm 0.68 (d, 3H), 1.03 (d, 3H), 1.60-1.70 (m, 1H), 2.23 (d, 1H), 2.42-2.50 (m, 1H), 2.66-2.75 (m, 1H), 2.77-2.88 (m, 2H), 3.07-3.16 (m, 2H), 3.55-3.62 (m, 1H), 7.46 (d, 2H), 7.67 (d, 2H), 7.80 (s, 4H). Mass Spectrum (ES-MS): M+1=384.2.

### Example 20

### Propane-2-sulfonic acid[trans-3-(2′-cyano-biphenyl-4-yl)-piperidin-4-yl]-amide

The title compound was prepared in a manner analogous to the preparation of propane-2-sulfonic acid[trans-3-(4′-cyano-biphenyl-4-yl)-piperidin-4-yl]-amide to give 90% of the product as a solid. 1H NMR (400 MHz, METHANOL-d₄) δ ppm 0,69 (d, 3H), 1.04 (d, 3H), 1.60-1.71 (m, 1H), 2.23 (d, 1H), 2.34-2.45 (m, 1H), 2.67-2.76 (m, 1H), 2.77-2.90 (m, 2H), 3.08-3.18 (m, 3H), 3.54-3.65 (m, 1H), 7.46-7.50 (m, 2H), 7.51-7.57 (m, 4H), 7.73 (t, 1H), 7.83 (d, 1H). Mass Spectrum (ES-MS): M+1=384.2.

### Example 21

### 4′-[trans-4-(Propane-2-sulfonylamino)-piperidin-3-yl]-biphenyl-2-carboxylic acid amide

The title compound was prepared in a manner analogous to the preparation of propane-2-sulfonic acid[trans-3-(4′-cyano-biphenyl-4-yl)-piperidin-4-yl]-amide to give 96% of the product as a solid. 1H NMR (400 MHz, METHANOL-d₄) δ ppm 0.73 (d, 3H), 1.06 (d, 3H), 1.60-1.70 (m, 1H), 2.23 (d, 1H), 2.36-2.45 (m, 1H), 2.63-2.71 (m, 1H), 2.82 (q, 3H), 3.06-3.16 (m, 3H), 3.53-3.62 (m, 1H), 7.34-7.38 (m, 4H), 7.39-7.46 (m, 4H), 7.48-7.55 (m, 2H). Mass Spectrum (ES-MS): M+1=402.2.

### Example 22

### Propane-2-sulfonic acid[trans-3-(2′-chloro-biphenyl-4-yl)-piperidin-4-yl]-amide

The title compound was prepared in a manner analogous to the preparation of propane-2-sulfonic acid[trans-3-(4′-cyano-biphenyl-4-yl)-piperidin-4-yl]-amide to give 96% of the product as a white solid. 1H NMR (400 MHz, METHANOL-d₄) δ ppm 0.72 (d, 3H), 1.03 (d, 3H), 1.60-1.71 (m, 1H), 2.21 (d, 1H), 2.28-2.38 (m, 1H), 2.64-2.72 (m, 1H), 2.76-2.89 (m, 2H), 3.12 (dt, 2H), 3.55-3.63 (m, 1H), 7.31-7.37 (m, 3H), 7.40 (s, 4H), 7.49 (d, 1H). Mass Spectrum (ES-MS): M+1=393.2.

### Example 23

### Propane-2-sulfonic acid[trans-3-(2′,5′-dichloro-biphenyl-4-yl)-piperidin-4-yl)]-amide

The title compound was prepared in a manner analogous to the preparation of propane-2-sulfonic acid[trans-3-(4′-cyano-biphenyl-4-yl)-piperidin-4-yl]-amide to give 97% of the product as a solid. 1H NMR (400 MHz, METHANOL-d₄) δ ppm 0.72 (d, 3H), 1.03 (d, 3H), 1.59-1.70 (m, 1H), 2.21 (d, 1H), 2.32-2.42 (m, 1H), 2.64-2.72 (m, 1H), 2.76-2.88 (m, 2H), 3.11 (ddd, 2H), 3.54-3.63 (m, 1H), 7.34 (t, 1H), 7.36-7.39 (m, 1H), 7.39-7.44 (m, 4H), 7.49 (d, 1H). Mass Spectrum (ES-MS): M+1=427.1.

### Example 24

### Propane-2-sulfonic acid[trans-3-(4′-cyano-biphenyl-4-yl)-1-methyl-piperidin-4-yl]-amide

A mixture of the TFA salt of propane-2-sulfonic acid[trans-3-(4′-cyano-biphenyl-4-yl)-piperidin-4-yl]-amide (10.4 mgs, 0.0209 mmols), paraformaldehyde (7.0 mgs, 0.230 mmols, i-Pr₂NEt (10 μL, 0.0574 mmols), NaBH(OAc)₃ (16.7 mgs, 0.0788 mmols) and methylene chloride (0.35 mL) was shaken in an 8 mL RB vial at room temperature until all starting material was consumed by LC/MS. The reaction was quenched with saturated aqueous bicarbonate solution and the organic layer was separated. The aqueous layer was extracted with methylene chloride and the combined organics were dried and concentrated to give a white foam that was purified by column chromatography on silica gel with 1:19 methanol:ethyl acetate. The combined product fractions were concentrated to give the product (4.5 mgs, 54%) as a clear glass. 1H NMR (400 MHz, CHLOROFORM-d) δ ppm 0.85 (d, 3H), 1.09 (d, 3H), 2,29-2.41 (m, 1H), 2.41-2.58 (m, 2H), 2.63-2.73 (m, 4H), 2.73-2.81 (m, 1H), 3.34-3.55 (m, 3H), 3.66-3.81 (m, 1H), 7.44 (d, 2H), 7.60 (d, 2H), 7.64-7.69 (m, 2H), 7.72-7.80 (m, 2H). Mass Spectrum (ES-MS): M+1=398.2.

### Example 25

### Propane-2-sulfonic acid(trans-3-biphenyl-4-yl-1-methyl-piperidin-4-yl)-amide

The title compound was prepared in a manner analogous to the procedure for propane-2-sulfonic acid[trans-3-(4′-cyano-biphenyl-4-yl)-1-methyl-piperidin-4-yl]-amide to give 45% of the product as a white solid after purification by HPLC under the following conditions: Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. 1H NMR (400 MHz, METHANOL-d₄) δ ppm 0.66 (d, 3H), 1.01 (d, 3H), 1.74-1.85 (m, 1H), 2.17-2.26 (m, 1H), 2.27-2.32 (m, 1H), 2.33 (s, 4H), 2.35-2.43 (m, 1H), 2.78-2.86 (m, 1H), 2.91-3.00 (m, 2H), 3.43-3.53 (m, 1H), 7.30-7.36 (m, 1H), 7.38-7.46 (m, 4H), 7.59 (d, 4H). Mass Spectrum (ES-MS): M+1=373.3.

### Example 26

### Propane-2-sulfonic acid(trans-3-biphenyl-4-yl-1-methanesulfonyl-piperidin-4-yl)-amide

A mixture of the TFA salt of propane-2-sulfonic acid(trans-3-biphenyl-4-yl-piperidin-4-yl)-amide (31 mgs, 0.066 mmols), methanesulfonyl chloride (7.7 μL, 0.0991 mmols), iPr₂NEt (28.6 μL, 0.164 mmols), and methylene chloride (1.0 mL) in an 8 mL vial was shaken at room temperature 3 hours at which point no starting material present LC/MS. The reaction was washed saturated aqueous bicarbonate solution, and the organic layer was dried and concentrated to give an off-white film that was purified by column chromatography on a Biotage 12M column in 1.1 ethyl acetate:heptane. The combined product fractions were concentrated to a white solid that was primarily the desired product with a minor impurity that corresponded to the mono-phenyl product by LC/MS analysis. The resulting solid was further purified by HPLC under the following conditions: Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. The concentrated product fraction gave the target compound (10.5 mgs, 37%) as a white solid. 1H NMR (400 MHz, METHANOL-d₄) δ ppm 0.67 (d, 3H), 1.03 (d, 3H), 1.74-1.85 (m, 1H), 2.28-2.34 (m, 1H), 2.36-2.45 (m, 1H), 2.80-2.87 (m, 1H), 2.88 (s, 3H), 2.97-3.09 (m, 2H), 3.58-3.67 (m, 1H), 3.73-3.85 (m, 2H), 7.31-7.37 (m, 1H), 7.41-7.47 (m, 4H), 7.61 (t, 4H). Mass Spectrum (ES-MS) M+1=437.1.

### Example 27

### Propane-2-sulfonic acid(trans-1-acetyl-3-biphenyl-4-yl-piperidin-4-yl -amide

A mixture of the TFA salt of propane-2-sulfonic acid(trans-3-biphenyl-4-yl-piperidin-4-yl)-amide (31 mgs, 0.066 mmols), acetyl chloride (7.1 μL, 0.099 mmols), iPr₂NEt (28.6 μL, 0.164 mmols), and methylene chloride (1.0 mL) in an 8 mL vial was shaken at room temperature 3 hours at which point no starting material was present by LC/MS. The reaction was washed with saturated aqueous bicarbonate solution, the organic layer was dried and concentrated to give an off-white film that was purified by column la chromatography on a Biotage 12M column in ethyl acetate. The combined product fractions were concentrated to give a clear oil that was primarily the desired product with a minor impurity that corresponded to the mono-phenyl product by LC/MS analysis and was further purified by HPLC under the following conditions: Waters SunFire C18 ODB Sum 19×100 mm column, gradient: 85% A:15% B to 100% B over 6 minutes with 2 minute hold (A=0.05% TFA in water, B=0.05% TFA in acetonitrile), flow rate: 35 mL/min. The concentrated product fraction gave the target compound (12.5 mgs, 48%) as a white solid. 1H NMR (400 MHz, METHANOL-d₄) δ ppm 0.67 (dd, 3H), 0.98-1.05 (m, 3H), 1.51-1.71 (m, 1H), 2.14 (d, 3H), 2.21-2.33 (m, 1H), 2.37-2.46 (m, 1H), 2.57-2.75 (m, 1H), 2.78-2.92 (m, 1H), 3.35-3.48 (m, 1H), 3.68-3.76 (m, 1H), 3.92-4.06 (m, 1H), 4.56-4.68 (m, 1H), 7.31-7.37 (m, 1H), 7.41-7.48 (m, 4H), 7.58-7.65 (m, 4H). Mass Spectrum (ES-MS) M+1=401.2.

### Example 28

### Propane-2-sulfonic acid[trans-1-acetyl-3-(4′-cyano-biphenyl-4-yl)-piperidin-4-yl]-amide

The title compound was prepared in a manner analogous to the procedure for propane-2-sulfonic acid(trans-1-acetyl-3-biphenyl-4-yl-piperidin-4-yl)-amide to give a 61% yield of the product as a clear glass after HPLC purification on a Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate, 35 mL/min. 1H NMR (400 MHz, METHANOL-d₄) δ ppm 0.69 (dd, 3H), 1.04 (d, 3H), 2.14 (d, 3H), 2.18-2.33 (m, 1H), 2.44-2.51 (m, 1H), 2.63 (t, 1H), 2.88 (t, 1H), 3.41 (d, 1H), 3.73 (br. s., 1H), 3.98 (dd, 1H), 4.54-4.68 (m, 1H), 7.51 (t, 2H), 7.69 (d, 2H), 7.81 (s, 4H). Mass Spectrum (ES-MS) M+1 426.2.

### Example 29

### Propane-2-sulfonic acid[trans-1-acetyl-3-(2′-cyano-biphenyl-4-yl)-piperidin-4-yl]-amide

The title compound was prepared in a manner analogous to the procedure for propane-2-sulfonic acid(trans-1-acetyl-3-biphenyl-4-yl-piperidin-4-yl)-amide to give a 47% yield of the product as a clear glass after column chromatography on a Biotage 12S column using ethyl acetate. 1H NMR (400 MHz, METHANOL-d₄) δ ppm 0.71 (t, 3H), 0.99-1.08 (m, 3H), 1.53-1.73 (m, 1H), 2.09-2.18 (m, 3H), 2.20-2.34 (m, 1H), 2.37-2.47 (m, 1H), 2.62-2.83 (m, 1H), 2.83-2.94 (m, 1H), 3.35-3.49 (m, 1H), 3.72-3.81 (m, 1H), 3.94-4.07 (m, 1H), 4.51-4.69 (m, 1H), 7.51-7.61 (m, 6H). 7.75 (t, 1H), 7.84 (d, 1H) ). Mass Spectrum (ES-MS) M+1=426.2.

### Preparation of 4-Hydroxy-5,6-dihydro-2H-pyran-3-carboxylic acid methyl ester

Sodium hydride (60% in mineral oil, 1.2 eq.) was washed with hexane to remove the mineral oil. It was then suspended in dimethyl carbonate (35 ml). The suspension was heated to 60° C. with stirring. Tetrahydro-4H-pyran-4-one (5.0 ml, 54 mmol) was added drop-wise through a syringe to the stirred suspension under N₂. A few drops of anhydrous methanol were then added to initiate the reaction. The resulting suspension was refluxed for 6 hours. Caution: The initial boiling was not smooth. The suspension went up one-third of the condenser. A 500-ml flask and a long condenser are suggested for this scale.

The reaction mixture was allowed to cool to room temperature. It was then transferred to a mixture of 2.5 M HCl (50 ml) and ethyl acetate (200 ml). The contents were extracted. The organic layer was separated. The aqueous layer (pH 6-7) was re-extracted with ethyl acetate (2×100 ml). The combined organic extracts were dried over Na₂SO₄ and concentrated in vacuo. The crude product was purified on a Biotage 40 M silica gel column eluting with hexane/EtOAc (90:10) to give the desired β-keto ester product (1.88 g, 22%) as an oil. ¹H-NMR indicated it exists mainly in enol form: 1H-NMR (400 MHz, CHCl₃): δ=11.7 (s, enol OH), 4.24 (t, 2H, enol form), 4.17-4.20 (m, keto form), 4.06-4.10 (m, keto form), 3.94-4.00 (m, keto form), 3.82 (t, 2H, enol form), 3.75 (s, keto form), 3.73 (s, 3H, enol form), 2.61-2.64 (m, keto form), 2.50-2.55 (m, keto form), 2.34-2.38 (m, 2H, enol form).

### Preparation of 4-Trifluoromethanesulfonyloxy-5,6-dihydro-2H-pyran-3-carboxylic acid methyl ester

4-Hydroxy-5,6-dihydro-2H-pyran-3-carboxylic acid methyl ester (1.19 g) was dissolved in DCM (23 ml). The solution was cooled in an ice-bath. 2,6-Di-t-butyl-4-methylpyridine (1.56 g, 1 equiv.) in DCM (2 ml) was added, followed by drop-wise addition of trifluoromethane sulfonate (Tf₂O) (1.27 ml, 1 equiv.) via a syringe. The resulting reaction mixture was stirred at 0° C. After 0.5 hours, the ice-bath was removed and the stir was continued for an additional 18 hours under N₂ at room temperature. The reaction mixture was filtered to remove the pyridinium triflate salt. The flask and the precipitate were washed with DCM. The filtrate was concentrated to a semi-solid. It was triturated with ether (total of 80 ml) and filtered to further remove the salt by-product. The filtrate was then concentrated to afford 2.11 g of the product as an oil (97%). 1H-NMR (400 MHz, CDCl₃): δ=4.44 (t, 2H), 3.88 (t, 2H), 3.81 (s, 3H), 2.51-2.54 (m, 2H).

### Preparation of Example 30

### Propane-2-sulfonic acid(trans-4-biphenyl-4-yl-tetrahydro-pyran-3-yl)-amide

### Preparation of 4-Biphenyl-4-yl-5,6-dihydro-2H-pyran-3-carboxylic acid methyl ester

4-Trifluoromethanesulfonyloxy-5,6-dihydro-2H-pyran-3-carboxylic acid methyl ester (543 mg, 1.87 mmol), potassium carbonate (628.1 mg, 2.4 equiv.) and 4-biphenylboronic acid (374.7 mg, 1 equiv.) were taken up in anhydrous THF (12 ml). The mixture was purged with N₂ for 20 minutes. Pd (PPh₃)₄ (56.5 mg, 0.03 equiv.) was added and the resulting reaction mixture was heated at 65° C. for 6 hours 15 minutes. Water (10 ml) was added. The mixture was extracted with EtOAc (3×30 ml). The combined organic layer was dried over Na₂SO₄ and concentrated. It was purified by column chromatography on a Biotage 40 M silica column with hexane/EtOAc (85:15) to afford the product (304 mg, 55% yield). 1H-NMR (400 MHz, CD₃OD): δ=7.58-7.63 (m, 4H), 7.42 (dt, 2H), 7.30-7.37 (m, 1H), 7.235 (dd, 2H), 4.40 (t, 2H), 3.89 (t, 2H), 3.48 (s, 3H), 2.52-2.54 (m, 2H).

### Preparation of trans-4-Biphenyl-4-yl-tetrahydro-pyran-3-carboxylic acid methyl ester

4-Biphenyl-4-yl-5,6-dihydro-2H-pyran-3-carboxylic acid methyl ester (470 mg) was dissolved in EtOAc (1 ml) and EtOH (9 ml). 10% Pd/C (50% by wt. water) (350 mg) was added. The resulting reaction mixture was hydrogenated under 50 psi H₂ pressure at room temperature for 21.5 hours. The catalyst was filtered through Celite. The filtrate was concentrated to afford a white solid. It was flashed through a Biotage 40 S silica column to afford the desired product (136 mg, isolated yield 29%). 1H-NMR (400 MHz, CDCl₃): δ=7.53-7.58 (m, 4H), 7.42 (t, 2H), 7.32-7.35 (m, 3H), 4.315 (d, 1H), 4.215 (dd, 1H), 3.79 (dd, 1H), 3.59 (dt, 1H), 3.53 (s, 3H), 3.10-3.15 (m, 1H), 2.95 (broad s, 1H), 2.80 (dq, 1H), 1.765 (broad d, 1H).

### Preparation of trans-4-Biphenyl-4-yl-tetrahydro-pyran-3-ylamine

trans-4-Biphenyl-4-yl-tetrahydro-pyran-3-carboxylic acid methyl ester (100 mg) was dissolved in EtOH (4 ml) with heating. Sodium ethoxide (23.2 mg, 1 equiv.) in EtOH (1 ml) was added to the hot solution slowly. The resulting reaction mixture was heated at reflux for 5 hours. A 1:1 mixture of starting ester and the corresponding acid was resulted, which was concentrated and re-dissolved in MeOH (0.7 ml) and further hydrolysed with powdered KOH (41.2 mg, 2.1 equiv.) in water (1.3 ml) at 65° C. for 2.5 hours, then at room temperature for 14 hours 35 minutes. The reaction mixture was washed with EtOAc (2×2 ml). The aq. layer was then acidified with 1 M HCl (1 ml) to pH 2 and extracted with EtOAc (3×2 ml) to afford the desired acid as a white solid (101.8 mg, 100%). APCl LCMS: Observed mass: 283.13 (M+H). LC/MS/UV purity: 100%. LC/MS/ELSD purity: 100%. 1H-NMR (400 MHz, CD₃OD): δ=7.52-7.58 (m, 4H), 7.40 (t, 2H), 7.27-7.34 (m, 3H), 4.17 (dd, 1H), 4.04 (dd, 1H), 3.51-3.61 (m, 2H), 3.06 (dt, 1H), 2.92 (dt, 1H), 1.77-1.86 (m, 2H).

The acid obtained above was suspended in anhydrous toluene (1.5 ml). TEA (84 ul, 2 equiv.) and DPPA (130 ul, 2 equiv.) were added. The contents were shaken at 80° C. for 2.5 hours. The reaction mixture was cooled to room temperature and concentrated. The residue was dissolved in THF (3 ml). 2 M NaOH (1.5 ml) was added. The contents were stirred at room temperature for 1 hour. 2 M NaOH (1 ml) was added. The mixture was extracted with EtOAc (3×2.5 ml). The combined organic layer was dried over Na₂SO₄ and concentrated in ˜2 ml in volume. It was flashed through a Biotage 40 S silica column with EtOAc/MeOH (95:5) to afford the desired product (97.3 mg, still contaminated with some (PhO)₂POOH). 1H-NMR (400 MHz, CD₃OD): δ=7.37-7.43 (m, 4H), 7.18-7.25 (m, 4H), 7.04 (t, 1H), 4.07 (dd, 1H), 4.01 (dd, 1H), 3.52 (dt, 1H), 3.23 (t, 1H), 3.13 (dt, 1H), 2.62 (dt, 1H), 1.90-1.95 (m, 1H), 1.805 (dd, 1H).

### Example 30

### Propane-2-sulfonic acid(trans-4-biphenyl-4-yl-tetrahydro-pyran-3-yl)-amide

Anhydrous DCE (0.4 ml) was added to trans-4-biphenyl-4-yl-tetrahydro-pyran-3-ylamine (25 mg in 0.1 ml of toluene/EtOAc). The resulting clear solution was cooled to 0° C. DBU (40 ul, 2.7 eq.) was added, followed by drop-wise addition of isopropylsulfonyl chloride (30 ul, 2.7 eq.). The resulting reaction mixture was stirred under N₂ from 0° C. to room temperature for 2.5 hours. It was purified by column chromatography on a silica gel column with hexane/EtOAc (70:30) to afford the desired product (16.3 mg, 45% yield). APCI LCMS: Observed mass: 360.16 (M+H). 1H-NMR (400 MHz, CDCl₃): δ=7.55 (t, 4H), 7.43 (t, 2H), 7.31-7.36 (m, 3H), 4.36 (dd, 1H), 4.03 (dd, 1H), 3.42-3.54 (m, 2H), 3.19 (t, 1H), 2.51-2.58 (m, 2H), 1.98-2.04 (ml, 1H), 1.885 (broad d, 1H), 1.07 (d, 3H), 0.61 (d, 3H).

### Preparation of 4-biphenyl-4-yl-5,6-dihydro-2H-pyran-3-carboxylic acid

4-Biphenyl-4-yl-5,6-dihydro-2H-pyran-3-carboxylic acid methyl ester (157.1 mg) was dissolved in dioxane (1 ml). Aqueous 5 M HCl (4 ml, 40 eq.) was added, followed by DME (1 ml). The resulting reaction mixture was shaken at 90° C. for 21.5 hours. The reaction mixture was extracted with EtOAc (3×5 ml). The combined organic layer was dried over Na₂SO₄ and concentrated. As it was concentrated, the desired product was precipitated from the solution in multiple batches. They were triturated with MeOH and the precipitates were collected. 81.8 mg of pure desired product was obtained as a white solid (54% isolated yield). APCI LCMS:. Observed mass: 281.12 (M=H). LCMS/UV purity: 95.48%. LCMS/ELSD purity: 100%. 1H-NMR (400 MHz, CD₃OD): δ=7.56-7.62 (m, 4H), 7.41 (t, 2H), 7.31-7.33 (m, 1H), 7.27 (d, 2H), 4.41 (t, 2H), 3.89 (t, 2H), 2.51-2.53 (m, 2H).

### Example 31

### 4-Biphenyl-4-yl-5,6-dihydro-2H-pyran-3-carboxylic acid ethyl ester

4-Trifluoromethanesulfonyloxy-5,6-dihydro-2H-pyran-3-carboxylic acid ethyl ester (256 mg, 0.84 mmol), potassium carbonate (282.4 mg, 2.4 equiv.) and 4-biphenylboronic acid (186.4 mg, 1.1 equiv.) were taken up in anhydrous THF (5.5 ml). The mixture was purged with N₂ for 10 minutes. Pd (PPh₃)₄ (27.9 mg, 0.03 equiv.) was added and the resulting reaction mixture was heated at 60° C. for 17 hours 10 minutes. Water (3 ml) was added. The mixture was extracted with EtOAc (3×10 ml). The combined organic layer was filtered and concentrated to afford 371.2 mg of a brownish residue. It was purified by column chromatography with hexane/EtOAc (85:15) to afford the desired product (115 mg, 44% yield). 1H-NMR (400 MHz, CDCl₃): δ=7.56-7.60 (m, 4H), 7.44 (t, 2H), 7.33-7.39 (m, 1H), 7.23 (d, 2H), 4.47 (t, 2H), 3.98 (q, 2H), 3.91 (t, 2H), 2.52-2.56 (m, 2H), 0.93 (t, 3H).

### Preparation of cis-4-biphenyl-4-yl-tetrahydro-pyran-3-carboxylic acid

4-Biphenyl-4-yl-5,6-dihydro-2H-pyran-3-carboxylic acid ethyl ester (140 mg) was dissolved in EtOAc (1 ml) and EtOH (5 ml). 10% Pd/C (50% by wt. water) (86.5 mg) was added. The resulting reaction mixture was hydrogenated under 50 psi H₂ pressure at room temperature for 18 hours. The catalyst was filtered. The filtrate was concentrated. LCMS indicated that the reaction was only halfway completed. It was re-dissolved in EtOH (12 ml). 10% Pd/C (50% by wt. water) (120 mg) was added. The resulting reaction mixture was again hydrogenated under 50 psi H₂ pressure at room temperature for 22 hours. The reaction was progressed to completion. The catalyst was filtered. The filtrate was concentrated to afford the desired cis ester intermediate, which was dissolved in DME (0.5 ml). 5 M HCl (2 ml) and dioxane (0.5 ml) were added. The resulting reaction mixture was shaken at 90° C. for 12 hours. It was cooled to room temperature and extracted with EtOAc (15 ml). The organic layer was washed with brine (5 ml), dried over Na₂SO₄ and concentrated to afford the desired product. APCI LCMS: Observed 283.13 (M+H). LC/MS/UV purity: 100%. LC/MS/ELSD purity: 100%. 1H-NMR (400 MHz, CD₃OD): δ=7.58 (d, 2H), 7.55 (d, 2H), 7.37-7.40 (m, 4H), 7.28 (t, 1H), 4.27 (dd, 1H), 4.14 (dd, 1H), 3.83 (dd, 1H), 3.62 (dt, 1H), 3.12-2.20 (m, 1H), 2.95 (broad s, 1H), 2.70-2.80 (m, 1H), 1.73 (broad d, 1H).

### Preparation of cis-4-biphenyl-4-yl-tetrahydro-pyran-3-ylamine

cis-4-Biphenyl-4-yl-tetrahydro-pyran-3-carboxylic acid (70 mg) was suspended in anhydrous toluene (1.5 ml). TEA (79 ul, 2.3 equiv.) and DPPA (122 ul, 2.2 equiv.) were added. The contents were shaken at 80° C. for 3 hours. The reaction mixture was cooled to room temperature and concentrated. The residue was dissolved in THF (2.8 ml). 2 M NaOH (1.4 ml) was added. The contents were stirred at room temperature for 1 hour. Toluene/EtOAc (1:1, 6 ml) and 2 M NaOH (1 ml) were added. The mixture was extracted. The organic layer was separated. The aq. layer was re-extracted with toluene/EtOAc (1:1, 6 ml). The combined organic layer was concentrated, re-dissolved in toluene (1.5 ml) and washed with 2 M NaOH (0.5 ml). The toluene layer was separated and the aq. layer was re-extracted with EtOAc (1.5 ml). Both layers were loaded onto a silica SPE cartridge and eluted with EtOAc first, then with EtOAc/MeOH (90:10) to afford the desired cis amine product, which was used without further purification.

### Example 31

### Propane-2-sulfonic acid(cis-4-biphenyl-4-yl-tetrahydro-pyran-3-yl)-amide

Anhydrous DCE (0.3 ml) was added to cis-4-biphenyl-4-yl-tetrahydro-pyran-3-ylamine (20 mg). The resulting clear solution was cooled to 0° C. DBU (37 ul, 3 eq.) was added, followed by isopropylsulfonyl chloride (27 ul, 3 eq.). The resulting reaction mixture was stirred under N₂ from 0° C. to room temperature for 2 hours 40 minutes. It was purified by preparative HPLC to afford the desired product (4.5 mg, 16% yield). APCI LCMS: Observed 360.16 (M+H). LC/MS/UV purity: 100%. LC/MS/ELSD purity: 100%. 1H-NMR (400 MHz, CDCl₃): δ=7.56 (t, 4H), 7.43 (t, 2H), 7.34-7.37 (m, 1H), 7.31 (d, 2H), 4.53 (d, 1H), 4.09-4.18 (m, 2H), 3.70-3.76 (m, 2H), 3.56 (dt, 1H), 3.115 (td, 1H), 2.13-2.18 (m, 1H), 1.78 (d, 1H), 1.00 (d, 3H), 0.79 (d, 3H).

### Preparation of Examples 32-38

4-Trifluoromethanesulfonyloxy-5,6-dihydro-2H-pyran-3-carboxylic acid methyl ester (886.3 mg, 3.0 mmol), potassium carbonate (973.6 mg, 2.3 equiv.) and 4-(benzyloxy)phenylboronic acid (756.6 mg, 1.1 equiv.) were taken up in anhydrous THF (25 ml). The mixture was purged with N₂ for 0.5 h. Pd(PPh₃)₄ (165.7 mg, 0.05 equiv.) was added and the resulting reaction mixture was heated at 65° C. for 17 hours under N₂. Water (20 ml) was added. The mixture was extracted with EtOAc (50 ml, 2×30 ml). The combined organic layer was filtered and concentrated. It was purified by column chromatography on a Biotage 40 M silica column with n-heptane/EtOAc (80:20) to afford 4-(4-benzyloxy-phenyl)-5,6-dihydro-2H-pyran-3-carboxylic acid methyl ester (640 mg, 66% yield). 1H-NMR (400 MHz, CDCl₃): δ=7.36-7.44 (m, 4H), 7.34-7.36 (m, 1H), 7.09 (d, 2H), 6.94 (d, 2H), 5.06 (s, 2H), 4.43 (t, 2H), 3.87 (t, 2H), 3.51 (s, 3H), 2.47-2.51 (m, 2H).

4-(4-Benzyloxy-phenyl)-5,6-dihydro-2H-pyran-3-carboxylic acid methyl ester (1.5015 g) was dissolved in EtOAc (10 ml) and EtOH (50 ml). 10% Pd/C (50% by wt. water) (1.1 g) was added. The resulting reaction mixture was hydrogenated under 50 psi H₂ pressure at room temperature for 23 hours 40 minutes. The catalyst was filtered. The filtrate was concentrated to afford 1.1989 g of crude cis-4-(4-hydroxy-phenyl)-tetrahydro-pyran-3-carboxylic acid methyl ester as a pinkish solid, which was directly used for the subsequent step without further purification. 1H-NMR (400 MHz, CD₃OD): δ=7.06 (d, 2H), 6.68 (d, 2H), 4.135 (d, 1H), 4.09 (d, 1H), 3.73 (dd, 1H), 3.56 (m, 1H), 3.45 (s, 3H), 3.04 (td, 1H), 2.86 (broad s, 1H), 2.62 (dq, 1H), 1.615 (dd, 1H).

Cis-4-(4-hydroxy-phenyl)-tetrahydro-pyran-3-carboxylic acid methyl ester (583.5 mg) was suspended in DCM (15 ml). Pyridinium p-toluene-sulfonate (PPTS, 122.4 mg, 0.2 equiv.) and 3,4-dihydro-2H-pyran (DHP, 650 ul, 2.9 equiv.) were added. The resulting clear solution was stirred at room temperature for 64 hours 15 minutes. It was purified by column chromatography on a Biotage 40 M silica column with n-heptane/EtOAc (80:20 to 50:50) to afford cis-4-[4-(tetrahydro-pyran-2-yloxy)-phenyl]-tetrahydro-pyran-3-carboxylic acid methyl ester as a white solid (553.1 mg, 70% isolated yield for two steps). 1H-NMR (400 MHz, CDCl₃): δ=7.16 (d, 2H), 6.97 (dd, 2H), 5.36 (m, 1H), 4.25 (d, 1H), 4.13 (dd, 1H), 3.89 (dt, 1H), 3.73 (dd, 1H), 3.51-3.60 (m, 2H), 3.50 (s, 3H), 3.015 (td, 1H), 2.84 (broad s, 1H), 2.73 (dq, 1H), 1.92-2.00 (m, 1H), 1.81-1.84 (m, 2H), 1.61-1.69 (m, 4H).

cis-4-[4-(Tetrahydro-pyran-2-yloxy)-phenyl]-tetrahydro-pyran-3-carboxylic acid methyl ester (545.6 mg) was dissolved in EtOH (10 ml). Sodium ethoxide (120 mg, 1 equiv.) in EtOH (5 ml) was added. The resulting reaction mixture was heated at reflux for 2 hours. It was concentrated to afford trans-4-[4-(tetrahydro-pyran-2-yloxy)-phenyl]-tetrahydro-pyran-3-carboxylic acid ethyl ester, which was directly used for the subsequent step without further purification.

trans-4-[4-(Tetrahydro-pyran-2-yloxy)-phenyl]-tetrahydro-pyran-3-carboxylic acid ethyl ester obtained above was dissolved in MeOH (4 ml). Powdered KOH (100 mg, 1.05 equiv.) in water (6 ml) was added. The resulting suspension was heated at 65° C. for 16 hours 40 minutes. The reaction mixture was washed with EtOAc (2×2 ml). The aq. layer was then acidified with 1 M HCl (2.3 ml) to pH 6 and extracted with EtOAc (3×20 ml). The combined organic layer was dried over Na₂SO₄ and concentrated to afford trans-4-[4-(tetrahydro-pyran-2-yloxy)-phenyl]-tetrahydro-pyran-3-carboxylic acid (517.3 mg, 99%). 1H-NMR (400 MHz, CDCl₃): δ=7.09 (d, 2H), 6.94 (d, 2H), 5.35 (m, 1H), 4.185 (dd, 1H), 4.10 (d, 1H), 3.89 (dt, 1H), 3.46-3.59 (m, 4H), 2.85 (dt, 1H), 2.84 (dt, 1H), 1.62-1.83 (m, 7H).

trans-4-[4-(Tetrahydro-pyran-2-yloxy)-phenyl]-tetrahydro-pyran-3-carboxylic acid obtained above was triturated with toluene (2×3 ml) and concentrated before use. It was then suspended in anhydrous toluene (7 ml). TEA (456 ul, 2 equiv.) and DPPA (707 ul, 2 equiv.) were added. The contents were shaken at 80° C. under N2 for 3 hours. The reaction mixture was cooled to room temperature and concentrated. The residue was dissolved in THF (15 ml). 2 M NaOH (8.2 ml, 10 equiv.) was added. The resulting suspension was stirred at room temperature for 1 hour. Water (2 ml) was added. The mixture was extracted with EtOAc (3×20 ml). The combined organic layer was dried over Na₂SO₄ and concentrated in ˜2 ml in volume. It was flashed through a Biotage 40 S silica column with EtOAc/1 M NH3 in MeOH (95:5) to afford trans-4-[4-(tetrahydro-pyran-2-yloxy)-phenyl]-tetrahydro-pyran-3-ylamine as a white solid (308 mg, 68%). 1H-NMR (400 MHz, CDCl₃): δ=7.14 (dd, 2H), 7.01 (dd, 2H), 5.38 (m, 1H), 4.015 (dt, 2H), 3.91 (dt, 1H), 3.585 (td, 1H), 3.46(t, 1H), 3.10 (t, 1H), 2.95 (dt, 1H), 2.33 (dt, 1H), 1.92-2.03 (m, 1H), 1.82-1.86 (m, 3H), 1.58-1.74 (m, 4H).

Anhydrous DCE (7 ml) was added to trans-4-[4-(tetrahydro-pyran-2-yloxy)-phenyl]-tetrahydro-pyran-3-ylamine (351.6 mg, 1.27 mmol). The resulting clear solution was cooled to 0° C. DBU (475 ul, 2.5 eq.) was added, followed by drop-wise addition of isopropylsulfonyl chloride (355 ul, 2.5 eq.) under N₂. The ice-bath was removed. The resulting reaction mixture was allowed to come to room temperature and stirred for 17 hours. It was purified by column chromatography with hexane/EtOAc (50:50) to afford Propane-2-sulfonic acid{trans-4-[4-(tetrahydro-pyran-2-yloxy)-phenyl]-tetrahydro-pyran-3-yl}-amide (315 mg, with partial loss of THP). 1H-NMR (400 MHz, CD₃OD): δ=7.22 (d, 2H), 6.99 (d, 2H), 5.40 (t, 1H), 4.10 (dd, 1H), 3.93 (dt, 1H), 3.84 (t, 1H), 3.56 (td, 1H), 3.42 (t, 1H), 3.37-3.42 (m, 1H), 3.16 (t, 1H), 2.56 (dt, 1H), 2.32-2.38 (m, 1H), 1.57-1.84 (m, 8H), 1.006 (d, 3H), 0.606 (d, 3H).

Propane-2-sulfonic acid{trans-4-[4-(tetrahydro-pyran-2-yloxy)-phenyl]-tetrahydro-pyran-3-yl}-amide (184.8 mg) was dissolved in MeOH (6 ml). Water (25 ul, 2.9 eq.) and PPTS (22.5 mg, 0.19 eq.) were added. The resulting reaction mixture was stirred at room temperature for 17 hours. MeOH was removed. Water (5 ml) was added. The aq. layer was extracted with EtOAc (20 ml, 2×10 ml). The combined organic layer was washed with water (5 ml), sat. NH4Cl (5 ml), dried over Na₂SO₄ and concentrated to afford propane-2-sulfonic acid[trans-4-(4-hydroxy-phenyl)-tetrahydro-pyran-3-yl]-amide as a white solid (139.8 mg, 97% yield). 1H-NMR (400 MHz, CD₃OD): δ=7.12 (d, 2H), 6.73 (d, 2H), 4.09 (dd, 1H), 3.93 (dd, 1H), 3.41 (t, 1H), 3.31-3.41 (m, 2H), 3.15 (t, 1H), 2.515 (dt, 1H), 2.34-2.41 (m, 1H), 1.87 (dq, 1H), 1.74 (dd, 1H), 1.01 (d, 3H), 0.62 (d, 3H).

Propane-2-sulfonic acid[trans4-(4-hydroxy-phenyl)-tetrahydro-pyran-3-yl]-amide (180 mg, 0.6 mmol) was suspended in anhydrous DCM (8 ml). 2,6-lutidine (119 ul, 1.7 eq.) and DMAP (10.8 mg, 0.15 eq.) were added. The contents were cooled to 0° C. Tf₂O (172 ul, 1.7 eq.) was added drop-wise through a syringe under N₂. The resulting reaction mixture was stirred from 0° C. to room temperature for 20.5 hours. Water (5 ml) was added. The contents were extracted. The organic layer was separated. The aq. layer was re-extracted with EtOAc (2×20 ml). The combined organic layer was washed with water (10 ml), brine (10 ml), dried over Na₂SO₄ and concentrated to afford 315 mg of a crude product, which was triturated with EtOAc/hexane (1:9, 2×10 ml) to afford trifluoro-methanesulfonic acid 4-[trans-3-(propane-2-sulfonylamino)-tetrahydro-pyran-4-yl]-phenyl ester. It was used without further purification. 1H-NMR (400 MHz, CD₃OD): δ=7.51 (d, 2H), 7.33 (d, 2H), 4.13 (dd, 1H), 3.96 (dd, 1H), 3.44 (m, 2H), 3.19 (t, 1H), 2,72 (dt, 1H), 2.36-2.39 (m, 1H), 1.93-1.99 (m, 1H), 1.83 (dd, 1H), 1.00 (d, 3H), 0.63 (d, 3H).

General procedure B: The aryl triflate (50 umol), aryl boronic acid (3 eq.), potassium phosphate (2.5 eq.) and potassium bromide (1.5 eq.) were taken up in 1,4-dioxane (0.5 ml). The contents were deoxygenated with N₂ for a few minutes. Pd (PPh₃)₄ (from Strem, 0.05 eq.) was added. The resulting reaction mixture was heated under reflux (100 to 105° C.) for 5 hours. For boronic acids that are poorly soluble in dioxane, co-solvent DMF (0.2 ml) was added to aid the solubilization. The reaction progress was checked by LCMS. If little desired product was seen, then additional aryl boronic acid (3 eq.) and Pd(PPh₃)₄ (0.05 eq.) were added and the reaction mixture was heated under reflux for an additional 5 hours. The reaction mixture was cooled to room temperature and purified with HPLC on a Waters SunFire C18 ODB 5 um 19×100 mm column, gradient: 85% A:15% B to 100% B over 6 minutes with 2 minute hold (A=0.05% TFA in water, B=0.05% TFA in acetonitrile), flow rate: 35 mL/min. Products werefurther cleaned up with CombiFlush RediSep silica flush column if necessary to obtain the desired product. The isolated yield ranges from 14 to 24%.

### Example 32

### Propane-2-sulfonic acid[trans-4-(4′-fluoro-biphenyl-4-yl)-tetrahydro-pyran-3-yl]-amide

1H-NMR (400 MHz, CDCl₃): δ=7.48-7.54 (m, 4H), 7.32 (d, 2H), 7.12 (t, 2H), 4.39 (dd, 1H), 4.05 (dd, 1H), 3.89 (d, 1H, SO₂NH proton), 3.50-3.58 (m, 1H), 3.47 (dt, 1H), 3.20 (t, 1H), 2.52-2.62 (m, 2H), 2.03 (dq, 1H), 1.895 (dd, 1H), 1.10 (d, 3H), 0.66 (d, 3H). LCMS-UV purity=82.6%, LCMS-ELSD purity=100%, calculated for C₂₀H₂₄FNO₃S: 377.1, M⁺ found to be 378.1.

### Example 33

### Propane-2-sulfonic acid[trans-4-(2′-ethoxy-biphenyl-4-yl)-tetrahydro-pyran-3-yl]-amide

1H-NMR (400 MHz, CDCl₃): δ=7.55 (d, 2H), 7.27-7.32 (m, 4H), 7.12 (t, 2H), 6.96-7.03 (m, 2H), 4.40 (dd, 1H), 4.02-4.07 (dd, 1H+q, 2H), 3.88 (d, 1H, SO₂NH proton), 3.43-3.52 (m, 2H), 3.20 (t, 1H), 2.51-2.60 (m, 2H), 2.04 (dq, 1H), 1.93 (dd, 1H), 1.33 (t, 3H), 1.10 (d, 3H), 0.62 (d, 3H). LCMS-UV purity=97%, LCMS-ELSD purity=100%, calculated for C₂₂H₂₉NO₄S: 403.2, M⁺ found to be 404.2.

### Example 34

### Propane-2-sulfonic acid[trans-4-(2′-cyano-biphenyl-4-yl)-tetrahydro-pyran-3-yl]-amide

1H-NMR (400 MHz, CDCl₃): δ=7.77 (d, 1H), 7.56 (d, 2H), 7.56 (d, 2H), 7.47 (d, 2H), 7.40 (d, 2H), 4.39 (dd, 1H), 4.06 (dd, 1H), 3.97 (d, 1H, SO₂NH proton), 3.57 (dd, 1H), 3.48 (dt, 1H), 3.20 (t, 1H), 2.54-2.63 (m, 2H), 2.05 (dq, 1H), 1.916 (dd, 1H), 1.10 (d, 3H), 0.71 (d, 3H). LCMS-UV purity=78%, LCMS-ELSD purity=100%, calculated for C₂₁H₂₄N₂O₃S: 384.2, M⁺ found to be 385.2.

### Example 35

### Propane-2-sulfonic acid[trans-4-(4′-cyano-biphenyl-4-yl)-tetrahydro-pyran-3-yl]-amide

1H-NMR (400 MHz, CDCl₃): δ=7.73 (d, 2H), 7.65 (d, 2H), 7.59 (d, 2H), 7.38 (d, 2H), 4.37 (dd, 1H), 4.05 (dd, 1H), 3.79 (d, 1H, SO₂NH proton), 3.50-3.59 (m, 1H), 3.47 (dt, 1H), 3.20 (t, 1H), 2.56-2.63 (m, 2H), 2.02 (dq, 1H), 1.91 (d, 1H), 1.11 (d, 3H), 0.72 (d, 3H). LCMS-UV purity=85%, LCMS-ELSD purity=100%, calculated for C₂₁H₂₄N₂O₃S: 384.2, M⁺ found to be 385.2.

### Example 36

### Propane-2-sulfonic acid[trans-4-(2′-chloro-biphenyl-4-yl)-tetrahydro-pyran-3-yl]-amide

1H-NMR (400 MHz, CDCl₃): δ=7.42-7.45 (m, 3H), 7.28-7.33(m, 5H), 4.40 (dd, 1H), 4.05 (dd, 1H), 3.90 (d, 1H, SO₂NH proton), 3.48-3.58 (m, 1H), 3.45 (dt, 1H), 3.20 (t, 1H), 2.49-2.59 (m, 2H), 2.04 (dq, 1H), 1.93 (dd, 1H), 1.09 (d, 3H), 0.68 (d, 3H). LCMS-UV purity=95%, LCMS-ELSD purity=100%, calculated for C₂₀H₂₄ClNO₃S: 393.1, M⁺ found to be 394.1.

### Example 37

### Propane-2-sulfonic acid[trans-4-(4′-methanesulfonyl-biphenyl-4-yl)-tetrahydro-pyran-3-yl]-amide

1H-NMR (400 MHz, CDCl₃); δ=8.00 (d, 2H), 7.73 (d, 2H), 7.60 (d, 2H), 7.38 (d, 2H), 4.36 (dd, 1H), 4.025 (dd, 1H), 3.885 (d, 1H, SO₂NH proton), 3.50-3.60 (m, 1H), 3.46 (dt, 1H), 3.20 (t, 1H), 2.57-2.62 (m, 2H), 2.01 (dq, 1H), 1.91 (dd, 1H), 1.10 (d, 3H), 0.71 (d, 3H). LCMS-UV purity=83%, LCMS-ELSD purity=100%, calculated for C₂₁H₂₇NO₅S₂: 437.1, M⁺ found to be 438.1.

### Example 38

### 4′-[trans-3-(Propane-2-sulfonylamino)-tetrahydro-pyran-4-yl]-biphenyl-4-carboxylic acid methylamide

1H-NMR (400 MHz, CDCl₃): δ=7.82 (d, 2H), 7.58-7.61 (m, 4H), 7.34 (d, 2H), 6.13 (broad m, 1H, CONH proton), 4.38 (dd, 1H), 4.14 (dd, 1H), 3.78 (d, 1H, SO₂NH proton), 3.50-3.60 (m, 1H), 3.47 (dt, 1H), 3.20 (t, 1H), 2.52-2.62 (m, 2H), 2.01 (dq, 1H), 1.90 (dd, 1H), 1.10 (d, 3H), 0.67 (d, 3H). LCMS-UV purity=96%, LCMS-ELSD purity=100%, calculated for C₂₂H₂₈N₂O₄S: 416.2, M⁺ found to be 417.2.

### Preparation of propane-2-sulfonic acid[trans-3-(4-nitro-phenyl)-piperidin-4-yl]-amide

tran-3-Phenyl-4-(propane-2-sulfonylamino)-piperidine-1-carboxylic acid tert-butyl ester was prepared from ethyl N-benzyl-3-oxo-piperidine carboxylate hydrochloride according to procedures described herein. ¹H-NMR (400 MHz, CDCl₃) 7.360-7.221 (m), 4.189 (bs), 3.815-3.772 (m), 3.598-3.524 (m), 3.089-3.059 (m), 2.539-2.484 (m), 2.461-2.392 (m), 2.354-2.311 (m), 1.623 (s), 1.441 (s), 1.391-1.264 (m), 1.246-1.238 (d), 1.177-1,084 (d); LC/MS purity=81.65%, ELSD/MS purity=100%, calculated for C₁₉H₃₀N₂O₄S 382.53, M⁺ found to be 383.3.

A solution of tran-3-phenyl-4-(propane-2-sulfonylamino)-piperidine-1-carboxylic acid tert-butyl ester (2.7 g, 7.06 mmol) in nitromethane (72 mL) was cooled to 0° C. A 33% solution of HNO3 (1.9 mL, 6 eq) in H2SO4 (5 mL, 13.5 eq) was cooled to 0° C. and added dropwise to the nitromethane solution. The solution was allowed to stir at at 0° C. for 2 hours. The resulting solution was quenched with ice and extracted with 3×50 mL EtOAc and 1M NaOH until pH=10-11 of aqueous layer. The organic layer was dried with sodium sulfate, filtered, and concentrated to give a crude yield of 1.846 g: ¹H-NMR (400 MHz, CD₃OD) 8.195-8.179 (m), 7.570-7.534 (m), 2.822-2.802 (m), 2.793-2.780 (m), 2.644-2.609 (m), 1.836-1.825 (m), 1.650-1.639 (m), 1.305-1.299 (d); LC/MS purity=47.88%, ELSD/MS purity=100%, calculated for C₁₄H₂₁N₃O₄S 327.4, M⁺ found to be 328.2.

### Preparation of propane-2-sulfonic acid[trans-1-acetyl-3-(4-nitro-phenyl)-piperidin-4-yl]-amide

To a solution of propane-2-sulfonic acid[trans-3-(4-nitro-phenyl)-piperidin-4-yl]-amide (956 mgs, 2.9 mmol) in DCM (13 mL) and triethylamine (489 μL, 1.2 eq) was added dropwise a solution of acetyl chloride (249 μL, 1.2 eq) and DCM (5.5 mL). The reaction mixture stirred for 3 hours at room temperature the resulting solution was extracted with 20 mL DCM, 10 mL 1M NaOH and 10 mL water. The aqueous layer was extracted 2×30 mL DCM. The organic layers were combined, dried with sodium sulfate, filtered, concentrated, and purified by flash chromatography (95:5 DCM/1M NH₃ in MeOH) to yield the product (104.8 mgs) ¹H-NMR (400 MHz, CD₃OD) 8.227-8.199 (m), 7.634-7.587 (m), 4.093-4.076 (d), 3.761-3.751 (m), 3.445-3.415 (m), 3.380-3.309 (m), 2.871-2.722 (m), 2.713-2.631 (m), 2.620-2.614 (m), 2.138-2.092 (d), 1.081-1.057 (m).

### Preparation of propane-2-sulfonic acid[trans-1-acetyl-3-(4-amino-phenyl)-piperidin-4-yl]-amide

37.59 mgs of propane-2-sulfonic acid[trans-1-acetyl-3-(4-nitro-phenyl)-piperidin-4-yl]-amide and a Pd/C catalyst in methanol were treated with 1 bar pressure of hydrogen at 70° C. The resulting solution was then purified by reverse phase HPLC to yield the product (18.8 mgs, 55%): ¹H-NMR (400 MHz, CD₃OD) 7.083-7.037 (m), 6.7136.684 (m), 4.478-4.445 (m), 3.574-3.539 (m), 2.752-2.688 (m), 2.392-2.318 (m), 2.109 (s), 2.061 (s), 1.995 (s), 1.240-1.203 (m), 1.041-1.024 (m), 0.695-0.678 (m); LC/MS purity=69.79%, calculated for C₁₆H₂₅N₃O₃S 339.46, M⁺ found to be 340.3.

### Example 39

### N-{4-[trans-1-Acetyl-4-(propane-2-sulfonylamino)-piperidin-3-yl]-phenyl}-benzamide

To a solution of propane-2-sulfonic acid[trans-1-acetyl-3-(4-amino-phenyl)-piperidin-4-yl]-amide (9.4 mg, 0.028 mmol) in DCE (312 μL) was added benzoyl chloride (3.85 μL, 1.2 eq) and DIPEA (5.79 μL, 1.2 eq). The solution was stirred capped for 1 hour. The resulting solution was quenched with 1 mL water and extracted with 3×3 mL DCM. The organic layers were combined, dried with sodium sulfate, filtered and concentrated to yield the product (5.6 mg, 46%): ¹H-NMR (400 MHz, CD₃OD) 8.160-8.143 (m), 8.139-7.735 (m), 7.717-7.565 (m), 7.550-7.480 (m), 7.380-7.332 (m), 2.442 (m), 2.132 (d), 2.089 (d), 1.057-1.040 (m); LC/MS purity=76.98%, ELSD/MS purity=100%, calculated for C₂₃H₂₉N₃O₄S 443.57, M⁺ found to be 444.1.

### Example 40

### N-{4-[trans-1-Acetyl-4-(propane-2-sulfonylamino)-piperidin-3-yl]-phenyl}-3,5-difluoro-benzamide

In a manner analogous to the preparation of compound N-{4-[trans-1-Acetyl-4-(propane-2-sulfonylamino)-piperidin-3-yl]-phenyl}-benzamide, propane-2-sulfonic acid[trans-1-acetyl-3-(4-amino-phenyl)-piperidin-4-yl]-amide (9.4 mg, 0.028 mmol) was treated with difluorobezoyl chloride (4.89 μL, 1.2 eq). Yield (4.2 mg, 32%): ¹H-NMR (400 MHz, CD₃OD) 7.662-7.361 (m), 2.132-2.088 (d), 1.273-1.037 (d); LC/MS purity=89.97%, ELSD/MS purity=100%, calculated for C₂₃H₂₇F₂N₃O₄S 479.55, M⁺ found to be 480.1.

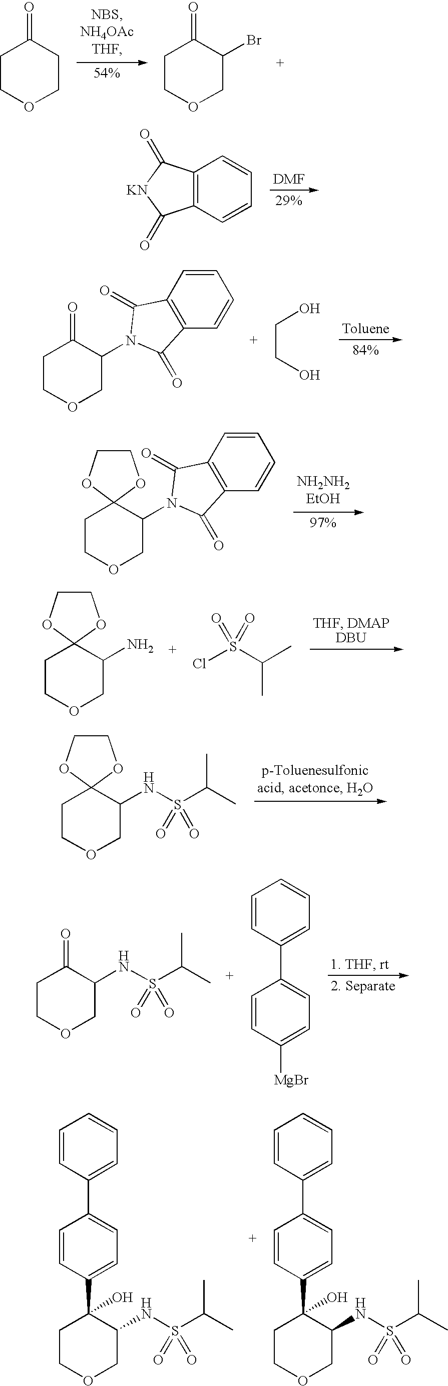

### Preparation of Propane-2-sulfonic acid((3S,4S)-rel-4-biphenyl-4-yl-4-hydroxytetrahydro-pyran-3-yl)-amide (Example 41) and Propane-2-sulfonic acid((3R,4S)-rel-4-biphenyl-4-yl-4-hydroxy-tetrahydro-pyran-3-yl)-amide (Example 42)

### Preparation of 3-bromo-tetrahydropyran-4-one

N-Bromosuccinimide (187 g, 1.05 mol, 1.05 eq) was added slowly to a suspension of tetrahydropyran-4-one (100 g, 1 mol) and ammonium acetate 7.7 g, 0.1 mol, 0.1 eq) in diethyl ether (500 mL) at 0° C. under N₂. The resulting mixture was stirred at room temperature overnight. The reaction mixture was filtered and the filtrate was concentrated. The crude product was purified using flash chromatography (eluting with ether in hexanes from 20% to 40%) to give 130 g (73%) of the title compound: ¹H NMR (CDCl₃, 400 MHz) δ 4.39 (ddd, 1H, J=7.9, 5.0, 1.2 Hz), 4.19 (dd, 1H, J=5.0, 1.2 Hz), 4.17-3.95 (m, 1H), 3.83-3.72 (m, 1H), 2.85-2.79 (m, 1H), 2.57-2.50 (m, 1H); ¹³C NMR (CDCl₃, 100 MHz) □ 198.4, 73.7, 68.4, 51.5, 41.4); GCMS m/z 180/178 (M+)

### Preparation of 2-(4-oxo-tetrahydro-2H-pyran-3-yl)isoindoline-1,3-dione

Potassium phthalimide (44.4 g, 240 mmol, 1.2 eq) was slowly added to a solution of 3-bromo-tetrahydropyran-4-one (35.8 g, 200 mmol) in anhydrous THF-DMF (3:1, 600 mL) at room temperature. The reaction mixture was stirred at rt for 4 days. The solids were filtered off and the filtrate was concentrated. The resulting crude product was purified using chromatography (eluting with 50-60% EtOAC in hexane) to give 25 g (50%) of the title compound: ¹H NMR (CDCl₃, 400 MHz) δ 7.86-7.82 (m, 2H), 7.76-7.70 (m, 2H), 4.95 (dd, 1H, J=10.4, 7.9 Hz), 4.37-4.21 (m, 3H), 3.88 (td, 1H, J=12.0, 2.9 Hz), 2.83-2.74 (m, 1H), 2.65-2.61 (m, 1H); ¹³C NMR (CDCl₃, 100 MHz) δ 199.3, 167.7, 134.5, 134.4, 132.0, 123.8, 123.7, 68.4, 67.6, 56.0; GCMS m/z 245 (M+)

### Preparation of 2-(1,4,8-trioxa-spiro[4.5]dec-6-yl)-isoindole-1,3-dione

A solution of 2-(4-oxo-tetrahydro-2H-pyran-3-yl)isoindoline-1,3-dione (24.5 g, 100 mol), p-toluenesulfonic acid monohydrate (0.95 g, 5 mmol, 0.05 eq), ethylene glycol (16.7 mL, 300 mmol, 3 eq) in toluene (500 mL) were heated at reflux with a Dean-Stark trap for 5 days. Upon completion of the reaction, the solution was cooled to room temperature and the toluene was removed under reduced pressure. The residue was partitioned between DCM (500 mL) and aqs satd NaHCO₃ (100 mL). The layers were separated and the organic layer was washed with brine, dried (Na₂SO₄), filtered and concentrated. The residue was triturated with ether (100 mL) to give 24 g (83%) of the title compound as a grey solid which was used without further purification: ¹H NMR (CDCl₃, 400 MHz) δ 7.76-7.71 (m, 2H), 7.68-7.63 (m, 2H), 4.66 (t, 1H, J=11.0 Hz), 4.39 (dd, 1H, J=11.2, 5.0 Hz), 3.89-3.79 (m, 4H), 3.72-3.56 (m, 3H), 1.90-1.82 (m, 1H), 1.78-1.73 (m, 1H); ¹³C NMR (CDCl₃, 100 MHz) □ 168.2, 134.3, 131.8, 123.5, 107.8, 66.7, 66.4, 65.2, 65.1, 54.0, 36.4; LCMS m/z 290.2 (M+1).

### Preparation of 1,4,8-trioxa-spiro[4.5]dec-6-ylamine

Hydrazine hydrate (20.2 mL, 415 mmol, 5 eq) was slowly added to a suspension of 2-(1,4,8-trioxa-spiro[4.5]dec-6-yl)-isoindole-1,3-dione (24 g, 83 mmol) in ethanol (600 mL) at room temperature. Upon completion of the addition, the contents were refluxed overnight. Heavy precipitation was observed. The reaction mixture was cooled to room temperature and the supernatant was decanted. The solids were washed with EtOAc (2×300 mL) and the combined organics were concentrated to a residue. The residue was triturated with EtOAc (200 mL) to remove the remaining byproduct as a solid. The solution was concentrated to an oil (11 g, 90% pure, 83% yield) that was used in the next step without any further purification: ¹H NMR (CDCl₃, 400 MHz) δ 3.98 (s, 2H), 3.80 (dd, 1H, J=11.2, 4.1 Hz), 3.76-3.70 (m, 1H), 3.65-3.59 (m, 1H), 2.80 (dd, 1H, J=7.9, 4.1 Hz), 2.10-1.90 (br s, 2H), 1.89-1.83 (m, 1H), 1.62-1.55 (m, 1H); LCMS m/z 160.0 (M+1).

### Preparation of propane-2-sulfonic acid(1,4,8-trioxa-spiro[4.5]dec-6-yl)-amide

Triethylamine (19.3 mL, 138.4 mmol, 2.0 eq), DMAP (8.4 g, 69.2 mmol, 1.0 eq) and iso-propylsulphonyl chloride (15.47 mL, 138.4 mmol, 2.0 eq) were added sequentially to a solution of 1,4,8-trioxa-spiro[4.5]dec6-ylamine (11 g, 69.2 mmol) in anhydrous DCM (400 mL) at 0° C. The contents were slowly warmed to room temperature and stirred overnight. The reaction mixture was quenched by the addition of aq. satd. NaHCO₃ (30 mL) and the layers were separated. The organic layer was washed with brine (30 mL), dried (Na₂SO₄) filtered, concentrated. The crude product was purified by chromatography (eluting with 40-70% EtOAc in hexanes) to give 2.1 g (11%) of the title compound as an oil: ¹H NMR (CDCl₃, 400 MHz) δ 4.48 (d, 1H, J=8.8Hz), 4.16-3.97 (m, 5H), 3.84-3.75 (m, 1H), 3.66-3.43 (m, 2H), 3.32-3.22 (m, 1H), 2.11 (s, 1H), 1.91-1.84 (m, 1H), 1.76-1.65 (m, 1H), 1.40-1.37 (m, 1H); GCMS m/z 266 (M+1).

### Preparation of N-(4-oxo-tetrahydro-2H-pyran-3-yl)propane-2-sulfonamide

A solution of propane-2-sulfonic acid(1,4,8-trioxa-spiro[4.5]dec-6-yl)-amide (2.1 g, 7.92 mmol) and PPTS (1.0 g) in acetone (80 mL)-water (34 mL) was refluxed overnight. No product formation was observed. Additional PTSA (2.0 g) was added into the flask and the resulting mixture was refluxed for 3 days. GC/MS analysis of the reaction mixture showed only 5% conversion. Conc. H₂SO₄ (5 mL) was added in several portions over 3 days until complete deprotection was observed. The volatiles were removed and the residue was dissolved in EtOAc (300 mL) and washed with aqs satd NaHCO₃ (5×40 mL). The organic layer was concentrated and the crude residue was purified using chromatography (eluting with 60-80% EtOAc in hexanes) to give 1.2 g (69%) of the title compound as an oil: ¹H NMR (CDCl₃, 400 MHz) δ 5.31-5.24 (m, 1H), 4.53-4.47 (m, 1H), 4.36-4.24 (m, 2H), 3.67-3.58 (m 1H), 3.31 (t, 1H, J=10.6Hz), 3.20-3.11 (m, 1H), 2.85-2.74 (m, 1H), 2.61-2.55 (m, 1H), 1.41-1.37 (m, 3H); GCMS m/z 222 (M+1).

### Preparation of propane-2-sulfonic acid((3S,4S)-rel-4-biphenyl-4-yl-4-hydroxytetrahydro-pyran-3-yl)-amide (Example 41) and propane-2-sulfonic acid((3R,4S)-rel-4-biphenyl-4-yl-4-hydroxy-tetrahydro-pyran-3-yl)-amide

Biphenylmagnesium bromide (0.5M solution, 12.0 mL, 6.0 mmol, 2eq.) was slowly added to a solution of N-(4-oxo-tetrahydro-2H-pyran-3-yl)propane-2-sulfonamide (0.66 g, 3.0 mmol) in anhydrous THF (20 mL) at room temperature and the resulting mixture was stirred for 24 hours. The reaction mixture was quenched with the addition of aqs satd NH₄Cl (10 mL) and extracted with EtOAc (2×200 mL). The combined organics were washed with brine, dried (Na₂SO₄), filtered and concentrated. The crude product was purified by chromatography (eluting with 60-70% EtOAc in hexane) to give 0.26 g (23%) of the title compounds as a mixture of isomers (˜2:1) as a white solid. This mixture was separated by HPLC using a Waters Sunfire 19×100 OBD C18 column with a 0.1% TFA modifier with the following method:

Time(min)/% Water/% Acetonitrile 0/50/50

1/50/50

8/80/80

9.5/5/95

10/5/95

This method provided 70 mg of propane-2-sulfonic acid((3S,4S)-rel-4-biphenyl-4-yl-4-hydroxytetrahydro-pyran-3-yl)-amide: ¹H NMR (CDCl₃, 400 MHz) δ 7.62-7.57 (m, 6H), 7.43 (t, 1H, J=7.5 Hz), 7.33 (t, 1H, J=7.5 Hz), 4.21 (dd, 1H, J=11.4, 1.5 Hz), 4.00-3.89 (m, 2H), 3.80-3.76 (m, 1H), 3.48 (s, 1H), 2.79-2.71 (m, 1H), 1.68 (d, 1H, J=13.7 Hz), 0.95 (d, 3H, J=7.05 Hz), 0.70 (d, 3H, J=7.05 Hz); LCMS m/z 374.2 (M+1).

and 3.6 mg of propane-2-sulfonic acid((3R,4S)-rel-4-biphenyl-4-yl-4-hydroxy-tetrahydro-pyran-3-yl)-amide: ¹H NMR (CDCl₃, 400 MHz) δ 7.65-7.57 (m, 4H), 7.48-7.44 (m, 2H), 7.40-7.36 (m, 1H), 7.27-7.24 (m, 1H), 7.18-7.16 (m, 1H), 4.48 (d, 1H, J=8.7 Hz), 4.15-4.12 (m, 1H), 3.92-3.79 (m, 2H), 2.51-2.43 (m, 1H), 1.74 (d, ₁H, J=14.9 Hz), 1.03 (d, 3H, J=6.6 Hz), 0.69 (d, 3H, J=6.6 Hz); LCMS m/z 374.2 (M+1).

### Example 43

### Propane-2-sulfonic acid(trans-1-acetyl-4-biphenyl-4-yl-piperidin-3-yl)-amide

The title compound was prepared in a manner analogous to the procedure for propane-2-sulfonic acid(trans-1-acetyl-3-biphenyl-4-yl-piperidin-4-yl)-amide to afford the product in 43% yield after purification by column chromatography on silica gel (95:5 EtOAc/MeOH). ¹H-NMR (400 MHz, CDCl₃) 7.592-7.534 (m), 7.464-7.425 (m), 7.380-7.343 (m), 4.779-4.745 (d), 4.482-4.443 (d), 3.949-3.929 (d), 2.996-2.937 (m), 2.659-2.567 (m), 2.199 (s), 1.994-1.963(m) 1.861-1.828 (m), 1.578 (s), 1.269-1.233 (m), 1.115-1.099 (d), 0.590-0.060 (d); LCMS-UV purity=86.91%, LCMS-ELSD purity=100%, calculated for C₂₁H₂₈N₂O₄S₂ 400.5, M⁺ found to be 401.2.

### Example 44

### N-{4-[trans-1-Methyl-4-(propane-2-sulfonylamino)-piperidin-3-yl]-phenyl}-benzamide

### Preparation of propane-2-sulfonic acid[trans-1-methyl-3-(4-nitro-phenyl)-piperidin-4-yl]-amide

Using a procedure analogous to the preparation of Example 2, propane-2-sulfonic acid[trans-3-(4-nitro-phenyl)-piperidin-4-yl]-amide (889 mg, 2.7 mmol) was converted to 93 mg of the title compound. ¹H-NMR (400 MHz, CD₃OD) 8.227-8.199 (m), 7.634-7.587 (m), 3.56-3.43 (m), 2.98-2.83 (m), 2.66-2.58 (m), 2.33 (s) 2.33-2.24 (m), 2.23-2.19 (m), 1.82-1.73 (m), 1.05-1.06 (d), 0.78-0.79 (d).

### Preparation of N-{4-[trans-1-Methyl-4-(propane-2-sulfonylamino)-piperidin-3-yl]-phenyl}-benzamide

Using procedures analogous to the preparation of Example 40, propane-2-sulfonic acid[trans-1-methyl-3-(4-nitro-phenyl)-piperidin-4-yl]-amide was converted to the title compound. ¹H-NMR (400 MHz, CD₃OD) 8.161-8.137 (d of d), 7.925-7.900 (d of d), 7.756-7.716 (m), 7.667-7.601 (m), 7.586-7.549 (m), 7.517-7.479 (m), 7.327-7.306 (d of d), 3.48-3.39 (m), 2.85-2.99 (m), 2.81-2.73 (m), 2.441-2.425(m), 2.325 (s), 2.22-2.18 (m), 1.81-1.72 (m), 1.273 (s), 1.052-1.035 (d), 0.720-0.703 (d); LCMS-UV purity=100%.

### Example 45

### 3,5-Difluoro-N-{4-[trans-1-methyl-4-(propane-2-sulfonylamino)-piperidin-3-yl]-phenyl}-benzamide

Using procedures analogous to the preparation of Example 40, propane-2-sulfonic acid[trans-1-methyl-3-(4-nitro-phenyl)-piperidin-4-yl]-amide was converted to the title compound. ¹H-NMR (400 MHz, CD₃OD) 7.745-7.723 (d of d), 7.58-7.54 (d of d), 7.395-7.373 (d of d), 7.37-7.28 (m), 3.79-3.68 (m), 3.61-3.50 (m), 2.91-3.01 (m), 2.39-2.48 (m), 1.83-1.99 (m), 1.052-1.035 (d), 0.720-0.703 (d); LCMS-UV purity=100%

In addition to the foregoing examples, the following are examples of compounds that are encompassed by the invention:

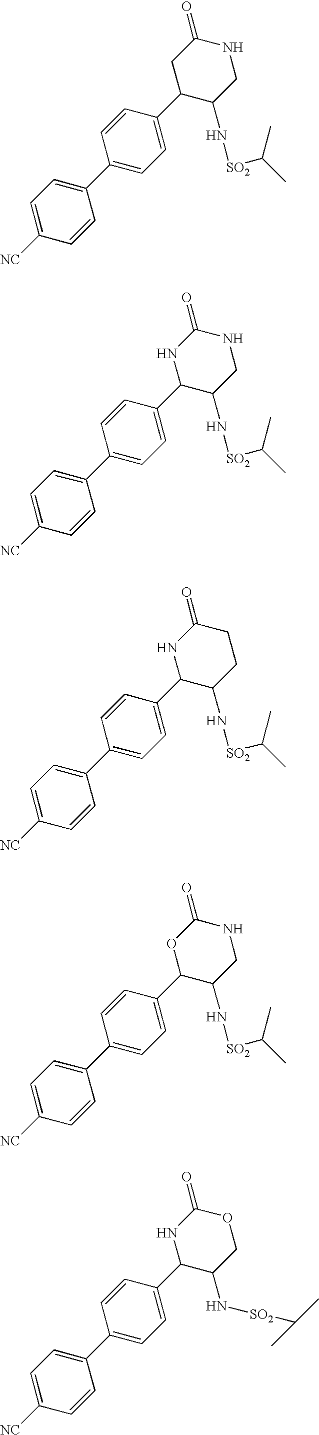

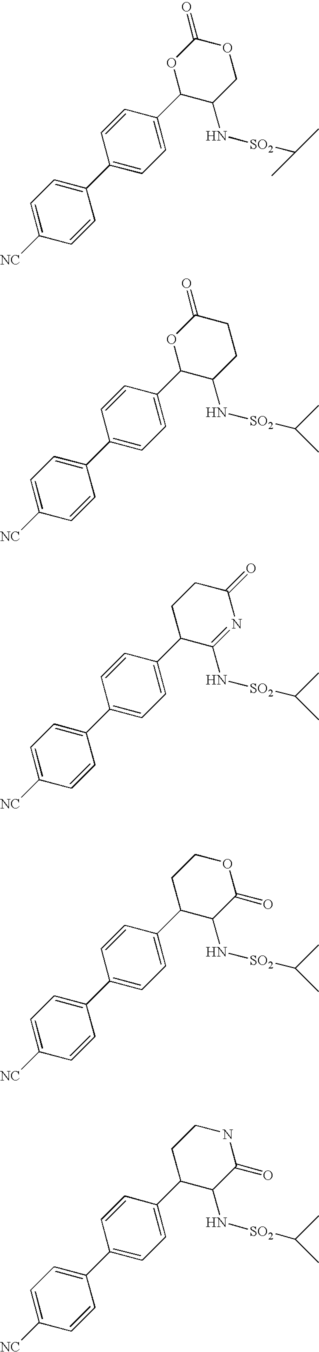

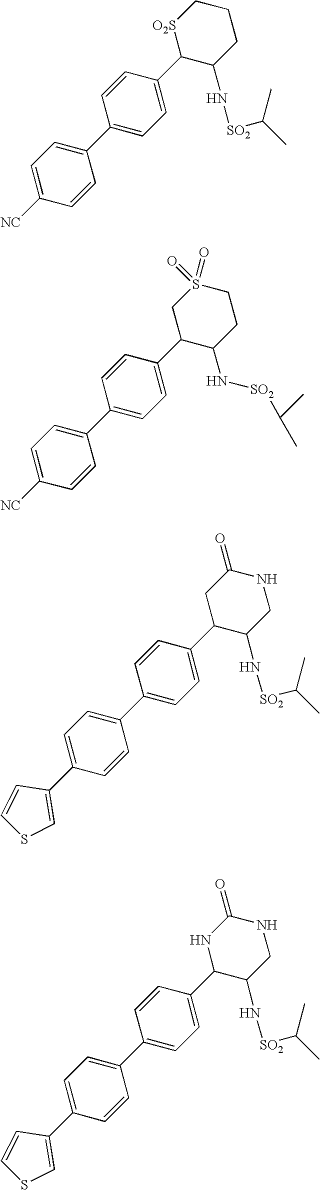

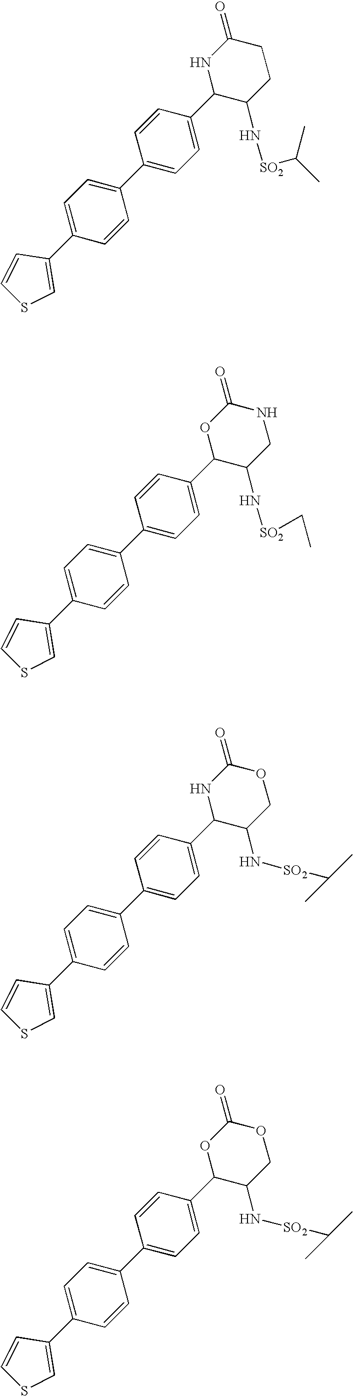

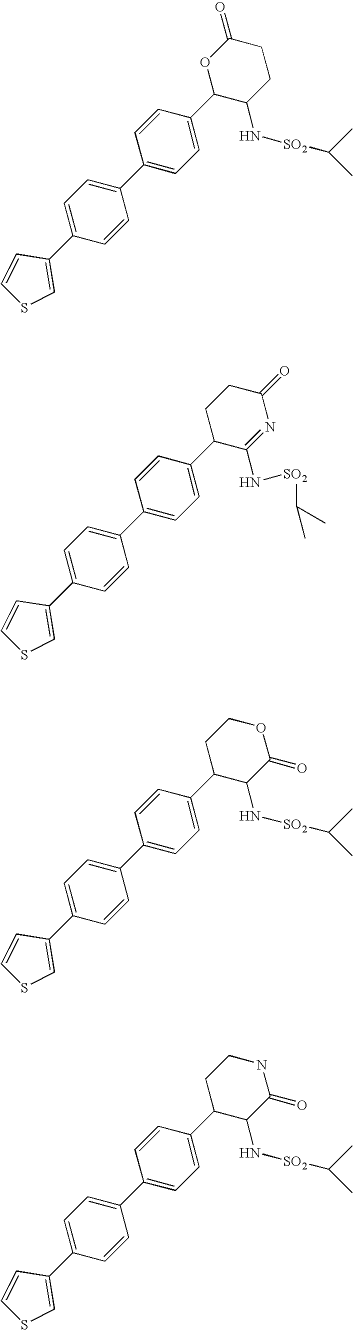

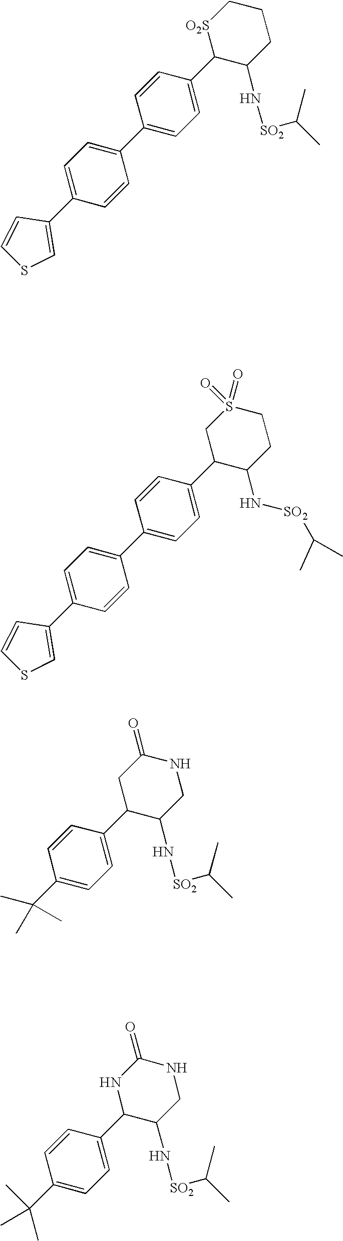

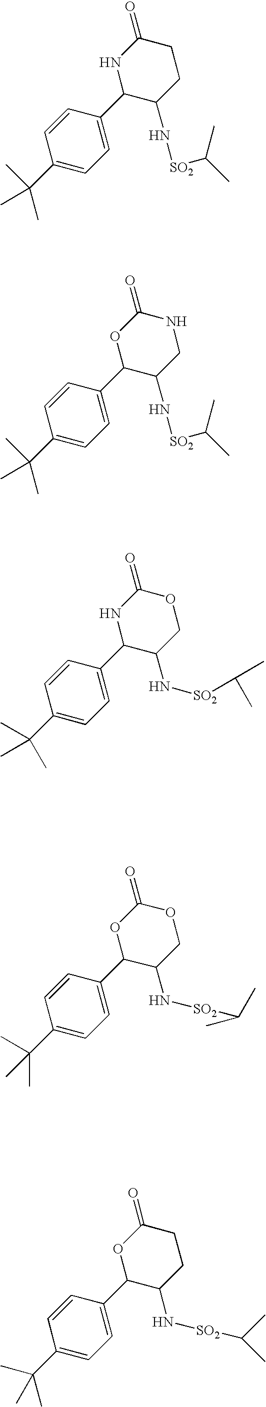

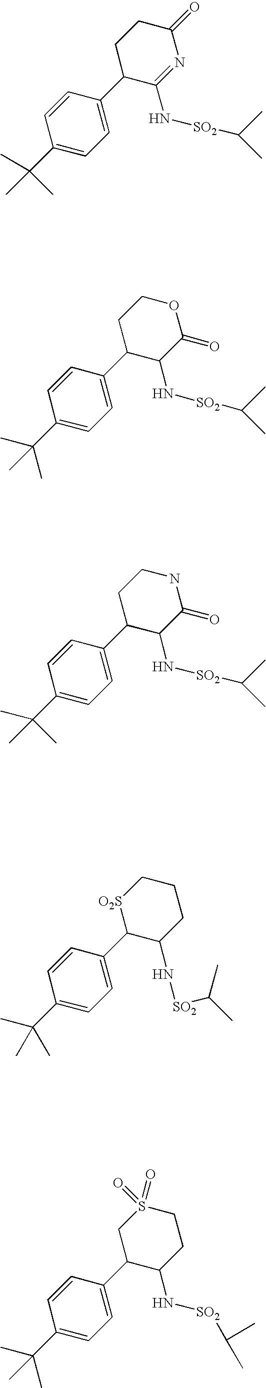

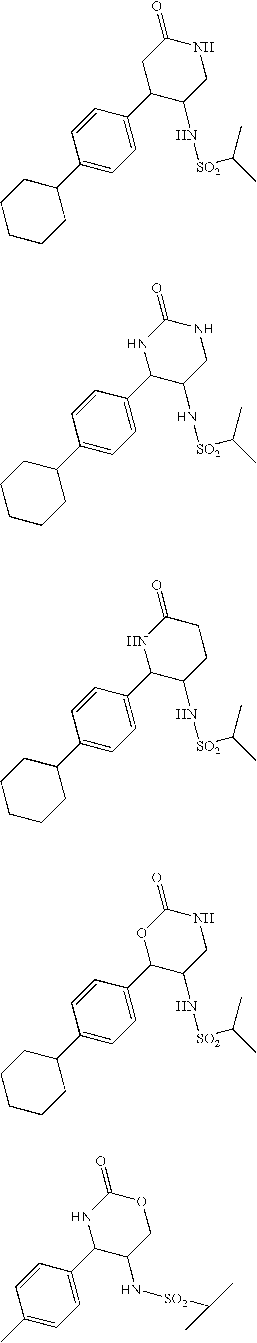

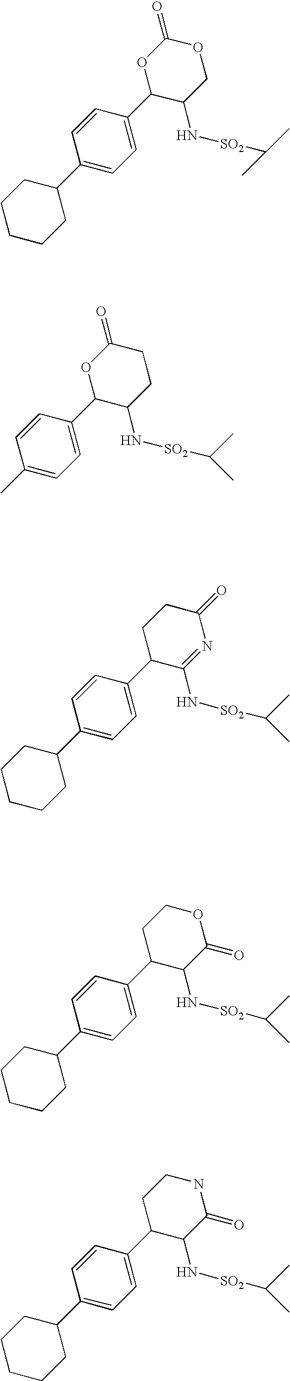

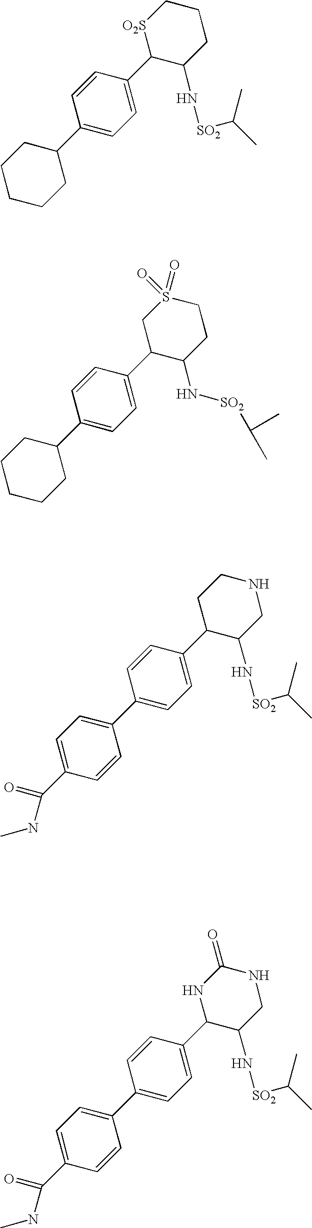

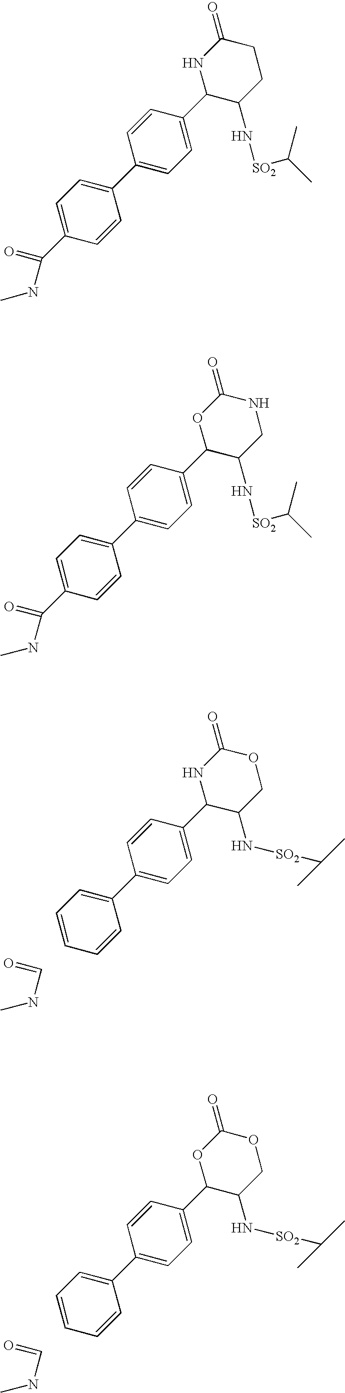

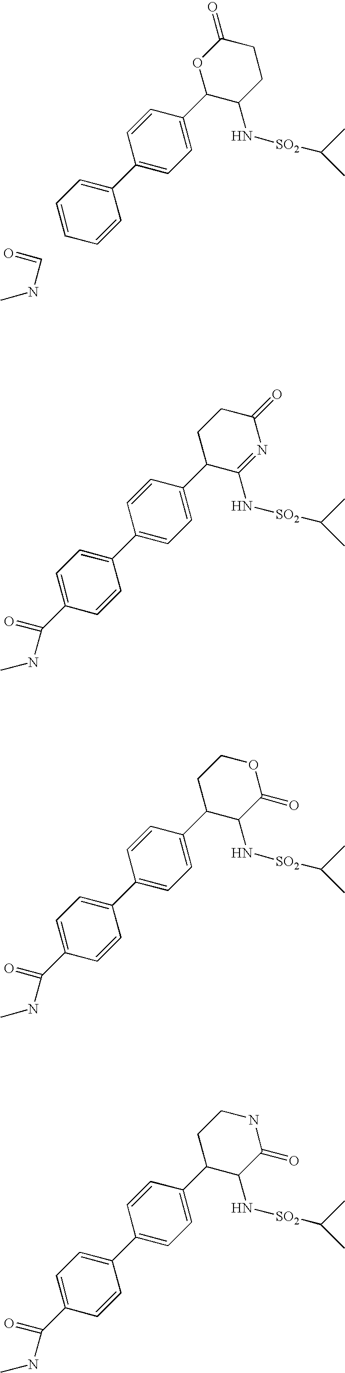

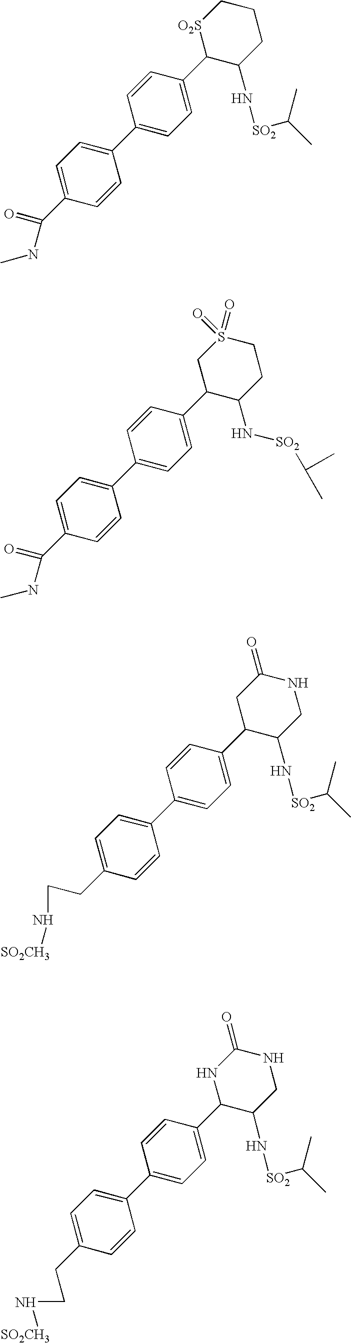

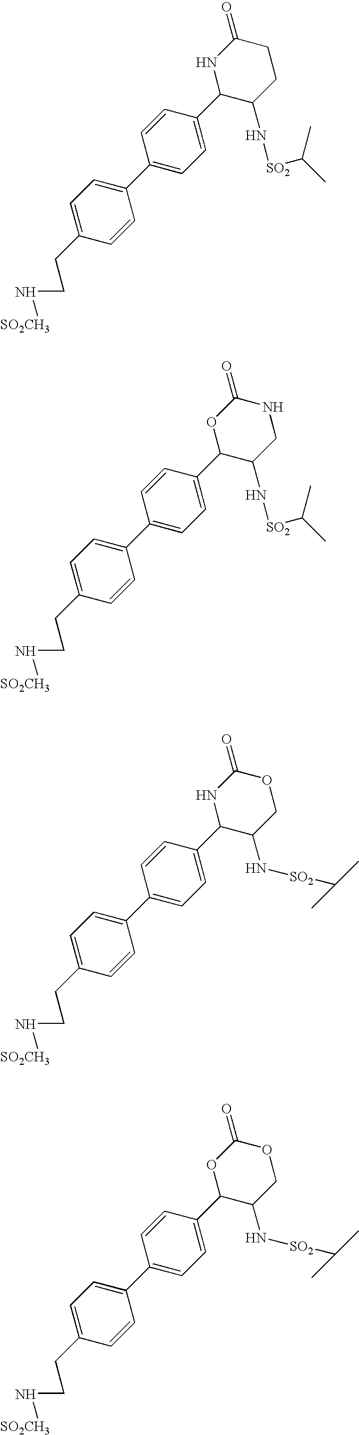

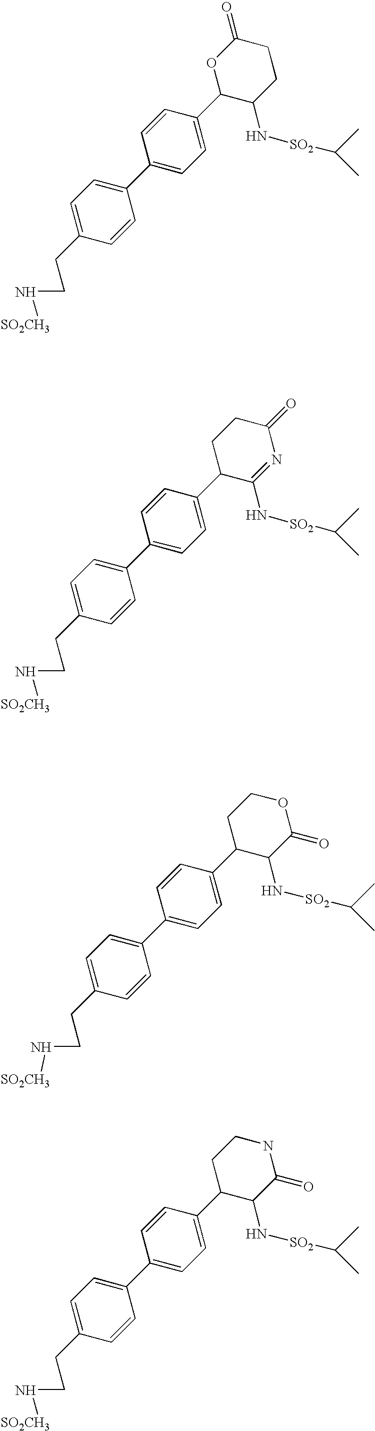

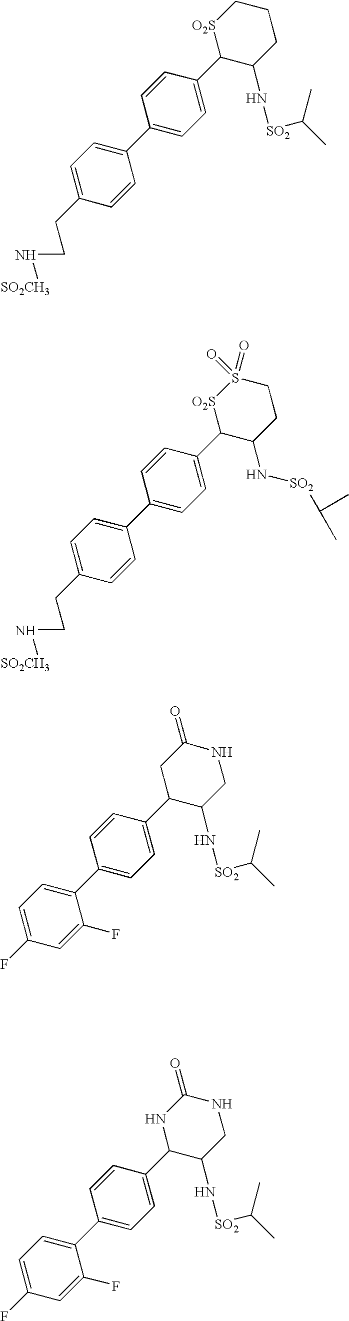

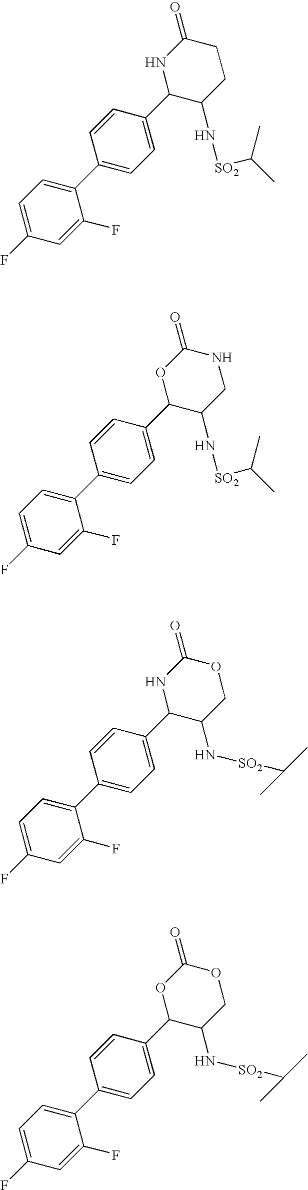

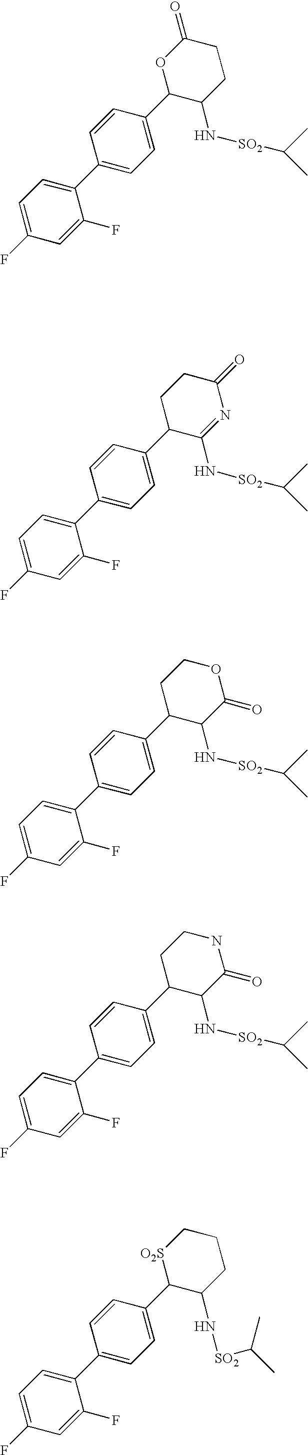

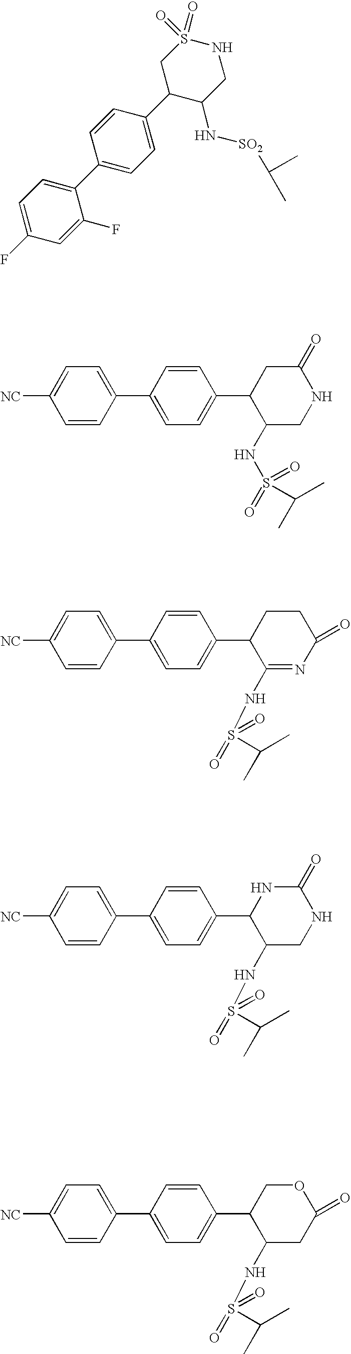

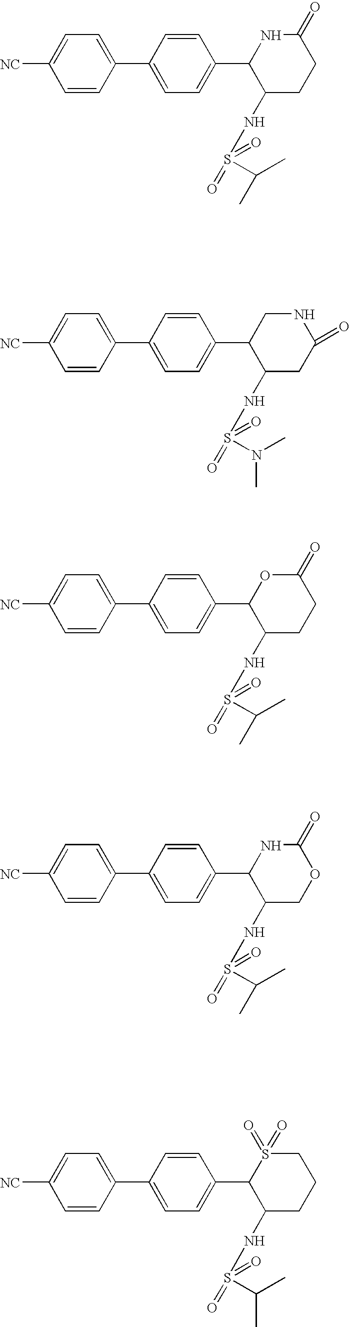

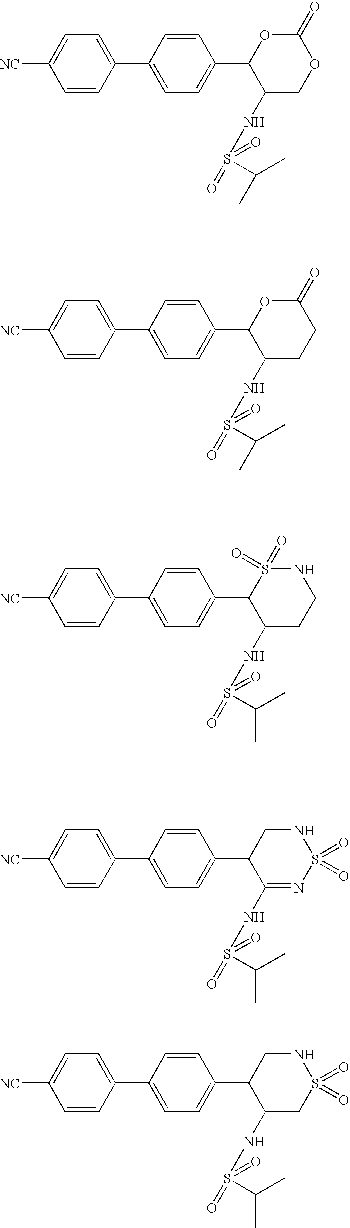

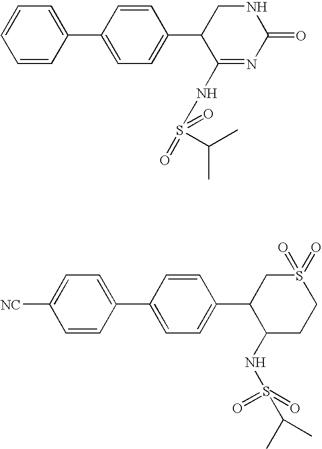

Table 1 shows examples of compounds according to the invention.

TABLE 1

| | | | G7330B | | | G7330B | ISIS name |
|---|---|---|---|---|---|---|---|
| | G7330A: | G7330A: | mean | G7330B | G7330B | (U): % | (used in |
| Ex # | Dose | % Effect | EC50 | (U): EC50 | (U): Dose | Effect | application) |
| 1 | 32.0 uM | 87.1% | 13.2 uM | 16.8 uM | 32.0 uM | 115% | Propane-2- |
| | | (79.0-95.2 | (7.80-22.5 | 11.1 uM | 32.0 uM | 132% | sulfonic acid |
| | | n = 4) | n = 3) | 12.5 uM | 32.0 uM | 92.5% | (trans-4- |
| | | | | | 32.0 uM | 85.0% | biphenyl-4-yl- |
| | | | | | 32.0 uM | 86.8% | piperidin-3- |
| | | | | | | | yl)-amide |
| 2 | 32.0 uM | 112% | 5.32 uM | 4.43 uM | 32.0 uM | 127% | Propane-2- |
| | | (103-121 | (3.12-9.08 | 5.97 uM | 32.0 uM | 141% | sulfonic acid |
| | | n = 4) | n = 4) | 3.78 uM | 32.0 uM | 134% | (trans-4- |
| | | | | 8.04 uM | 32.0 uM | 142% | biphenyl-4-yl- |
| | | | | | 32.0 uM | 151% | 1-methyl- |
| | | | | | 32.0 uM | 173% | piperidin-3- |
| | | | | | 32.0 uM | 180% | yl)-amide |
| 3 | | | >4.20 uM | 16.5 uM | 32.0 uM | 99.8% | Propane-2- |
| | | | (2.62-6.75 | 11.7 uM | 32.0 uM | 94.3% | sulfonic acid |
| | | | n = 17) | >32.0 uM | 32.0 uM | 83.1% | ((3R,4R)-4- |
| | | | | 2.03 uM | 32.0 uM | 129% | biphenyl-4-yl- |
| | | | | 3.43 uM | 32.0 uM | 98.9% | 1-methyl- |
| | | | | 4.19 uM | 32.0 uM | 119% | piperidin-3- |
| | | | | 4.57 uM | | | yl)-amide |
| | | | | 16.2 uM | | | |
| | | | | 2.78 uM | | | |
| | | | | 1.20 uM | | | |
| | | | | 1.50 uM | | | |
| | | | | 2.97 uM | | | |
| | | | | 3.28 uM | | | |
| | | | | 1.69 uM | | | |
| | | | | 3.00 uM | | | |
| | | | | 2.24 uM | | | |
| | | | | 5.39 Um | | | |
| 4 | | | >32.0 uM | >32.0 uM | 32.0 uM | 4.29% | Propane-2- |
| | | | (n = 4) | >32.0 uM | 32.0 uM | 2.21% | sulfonic acid |
| | | | | >32.0 uM | 32.0 uM | 2.27% | ((3S,4S)-4- |
| | | | | >31.6 uM | 32.0 uM | 2.45% | biphenyl-4-yl- |
| | | | | | 32.0 uM | 2.50% | 1-methyl- |
| | | | | | 32.0 uM | 6.32% | piperidin-3- |
| | | | | | 32.0 uM | 2.96% | yl)-amide |
| | | | | | 32.0 uM | 5.62% | |
| | | | | | 32.0 uM | 0.525% | |
| 5 | | | >31.6 uM | >31.6 uM | 31.6 uM | 76.3% | Propane-2- |
| | | | | | 31.6 uM | 78.8% | sulfonic acid |
| | | | | | | | ((3R,4R)-4- |
| | | | | | | | biphenyl-4-yl- |
| | | | | | | | 1-ethyl- |
| | | | | | | | piperidin-3- |
| | | | | | | | yl)-amide |
| 6 | | | >31.6 uM | >31.6 uM | 31.6 uM | 0.710% | Propane-2- |
| | | | | | 31.6 uM | −1.26% | sulfonic acid |
| | | | | | 31.6 uM | 0.0628% | (trans-4- |
| | | | | | | | biphenyl-4-yl- |
| | | | | | | | 1-methane- |
| | | | | | | | sulfonyl- |
| | | | | | | | piperidin-3- |
| | | | | | | | yl)-amide |
| 7 | 32.0 uM | −0.867% | | | 31.6 uM | −0.0209% | Propane-2- |
| | | (−1.95-0.221 n = 4) | | | 31.6 uM | −0.612% | sulfonic acid |
| | | | | | 31.6 uM | −1.41% | (cis-4- |
| | | | | | | −1.42% | biphenyl-4-yl- |
| | | | | | | | 1-methyl- |
| | | | | | | | piperidin-3- |
| | | | | | | | yl)-amide |
| 8 | 32.0 uM | 56.3% | >32.0 uM | >32.0 uM | 32.0 uM | 62.1% | N-(trans-4- |
| | | (48.6-64.0 | (n = 2) | >32.0 uM | 32.0 uM | 46.9% | Biphenyl-4-yl- |
| | | n = 4) | | | 32.0 uM | 54.7% | piperidin-3- |
| | | | | | 32.0 uM | 34.7% | yl)- |
| | | | | | 32.0 uM | 36.6% | methanesulfonamide |
| | | | | | 32.0 uM | 38.0% | |
| 9 | 32.0 uM | 59.1% | >32.0 uM | >32.0 uM | 32.0 uM | 73.4% | Ethanesulfonic |
| | | (54.6-63.5 | | | 32.0 uM | 60.8% | acid (trans- |
| | | n = 4) | | | 32.0 uM | 66.9% | 4-biphenyl-4- |
| | | | | | | | yl-piperidin-3- |
| | | | | | | | yl)-amide |
| 10 | 32.0 uM | 11.2% | >32.0 uM | >32.0 uM | 32.0 uM | 23.4% | Propane-1- |
| | | (7.05-15.3 | (n = 2) | >32.0 uM | 32.0 uM | 19.5% | sulfonic acid |
| | | n = 4) | | | 32.0 uM | 18.5% | (trans-4- |
| | | | | | 32.0 uM | 5.64% | biphenyl-4-yl- |
| | | | | | 32.0 uM | 6.55% | piperidin-3- |
| | | | | | 32.0 uM | 7.27% | yl)-amide |
| 11 | 32.0 uM | 2.34% | | | 32.0 uM | 1.55% | Ethanesulfonic |
| | | (1.30-3.39 n = 4) | | | 32.0 uM | 3.14% | acid (cis-4- |
| | | | | | 32.0 uM | 2.25% | biphenyl-4-yl- |
| | | | | | 32.0 uM | 2.45% | piperidin-3- |
| | | | | | | | yl)-amide |
| 12 | 32.0 uM | 0.110% | | | 32.0 uM | 0.428% | Propane-1- |
| | | (−0.338-0.558 n = 4) | | | 32.0 uM | −0.204% | sulfonic acid |
| | | | | | 32.0 uM | −0.02059% | (cis-4- |
| | | | | | 32.0 uM | 0.243% | biphenyl-4-yl- |
| | | | | | | | piperidin-3- |
| | | | | | | | yl)-amide |
| 13 | 32.0 uM | 43.7% | >32.0 uM | >32.0 uM | 32.0 uM | 46.2% | 2,2,2- |
| | | (37.2-50.1 | (n = 2) | >32.0 uM | 32.0 uM | 59.8% | Trifluoro- |
| | | n = 4) | | | 32.0 uM | 48.9% | ethanesulfonic |
| | | | | | 32.0 uM | 30.2% | acid (trans- |
| | | | | | 32.0 uM | 28.1% | 4-biphenyl-4- |
| | | | | | 32.0 uM | 27.9% | yl-piperidin-3- |
| | | | | | | | yl)-amide |
| 14 | 32.0 uM | 3.36% | >32.0 uM | >32.0 uM | 32.0 uM | 4.61% | Cyclopropanesulfonic |
| | | (2.50-4.23 | (n = 2) | >32.0 uM | 32.0 uM | 2.97% | acid |
| | | n = 4) | | | 32.0 uM | 3.26% | (trans-4- |
| | | | | | 32.0 uM | 0.0952% | biphenyl-4-yl- |
| | | | | | 32.0 uM | 0.150% | piperidin-3- |
| | | | | | 32.0 uM | 1.02% | yl)-amide |
| 15 | | | 3.64 uM | 2.84 uM | 32.0 uM | 108% | Propane-2- |
| | | | (1.54-8.63 | 3.14 uM | 32.0 uM | 109% | sulfonic acid |
| | | | n = 3) | 5.42 uM | 32.0 uM | 101% | (trans-3- |
| | | | | | | | biphenyl-4-yl- |
| | | | | | | | tetrahydro- |
| | | | | | | | pyran-4-yl)- |
| | | | | | | | amide |
| 16 | | | >18.2 uM | 10.4 uM | 32.0 uM | 153% | Ethanesulfonic |
| | | | (0.0156-21100 | >31.6 uM | 32.0 uM | 141% | acid (trans- |
| | | | n = 2) | >10.0 uM | 32.0 uM | 140% | 3-biphenyl-4- |
| | | | | | | | yl-tetrahydro- |
| | | | | | | | pyran-4-yl)- |
| | | | | | | | amide |
| 17 | | | 7.17 uM | 7.17 uM | 32.0 uM | 150% | Propane-2- |
| | | | | | 32.0 uM | 138% | sulfonic acid |
| | | | | | 32.0 uM | 125% | (cis-3- |
| | | | | | | | biphenyl-4-yl- |
| | | | | | | | tetrahydro- |
| | | | | | | | pyran-4-yl)- |
| | | | | | | | amide |
| 18 | | | >32.0 uM | >32.0 uM | 32.0 uM | 5.77% | Propane-2- |
| | | | | | 32.0 uM | 3.59% | sulfonic acid |
| | | | | | 32.0 uM | 3.97% | (trans-3- |
| | | | | | | | biphenyl-4-yl- |
| | | | | | | | piperidin-4- |
| | | | | | | | yl)-amide |
| 19 | | | >32.0 uM | >32.0 uM | 32.0 uM | 6.07% | Propane-2- |
| | | | | | 32.0 uM | 7.12% | sulfonic acid |
| | | | | | 32.0 uM | 6.19% | [trans-3-(4′- |
| | | | | | | | cyano- |
| | | | | | | | biphenyl-4- |
| | | | | | | | yl)-piperidin- |
| | | | | | | | 4-yl]-amide |
| 20 | | | >32.0 uM | >32.0 uM | 32.0 uM | 39.9% | Propane-2- |
| | | | (n = 5) | >31.6 uM | 32.0 uM | 48.6% | sulfonic acid |
| | | | | >31.6 uM | 32.0 uM | 44.4% | [trans-3-(2′- |
| | | | | >31.6 uM | | | cyano- |
| | | | | >31.6 uM | | | biphenyl-4- |
| | | | | | | | yl)-piperidin- |
| | | | | | | | 4-yl]-amide |
| 21 | | | >32.0 uM | >32.0 uM | 32.0 uM | 0.573% | 4′-[trans-4- |
| | | | (n = 2) | >31.6 uM | 32.0 uM | 0.266% | (Propane-2- |
| | | | | | 32.0 uM | 0.149% | sulfonylamino)- |
| | | | | | | | piperidin-3- |
| | | | | | | | yl]-biphenyl- |
| | | | | | | | 2-carboxylic |
| | | | | | | | acid amide |
| 22 | | | >32.0 uM | >32.0 uM | 32.0 uM | 18.9% | Propane-2- |
| | | | (n = 4) | >31.6 uM | 32.0 uM | 23.3% | sulfonic acid |
| | | | | >31.6 uM | 32.0 uM | 17.1% | [trans-3-(2′- |
| | | | | >31.6 uM | | | chloro- |
| | | | | | | | biphenyl-4- |
| | | | | | | | yl)-piperidin- |
| | | | | | | | 4-yl]-amide |
| 23 | | | >32.0 uM | >32.0 uM | 32.0 uM | 10.1% | Propane-2- |
| | | | (n = 2) | >31.6 uM | 32.0 uM | 9.71% | sulfonic acid |
| | | | | | 32.0 uM | 6.91% | [trans-3-(2′,5′- |
| | | | | | | | dichloro- |
| | | | | | | | biphenyl-4- |
| | | | | | | | yl)-piperidin- |
| | | | | | | | 4-yl]-amide |
| 24 | | | >31.6 uM | >31.6 uM | 31.6 uM | 14.5% | Propane-2- |
| | | | | | 31.6 uM | 19.9% | sulfonic acid |
| | | | | | 31.6 uM | 12.7% | [trans-3-(4′- |
| | | | | | | | cyano- |
| | | | | | | | biphenyl-4- |
| | | | | | | | yl)-1-methyl- |
| | | | | | | | piperidin-4- |
| | | | | | | | yl]-amide |
| 25 | | | >32.0 uM | >32.0 uM | 32.0 uM | −0.323% | Propane-2- |
| | | | | | 32.0 uM | 0.295% | sulfonic acid |
| | | | | | 32.0 uM | 2.72% | (trans-3- |
| | | | | | | | biphenyl-4-yl- |
| | | | | | | | 1-methyl- |
| | | | | | | | piperidin-4- |
| | | | | | | | yl)-amide |
| 26 | | | >32.0 uM | >32.0 uM | 32.0 uM | 13.3% | Propane-2- |
| | | | | | 32.0 uM | 10.8% | sulfonic acid |
| | | | | | 32.0 uM | 9.95% | (trans-3- |
| | | | | | | | biphenyl-4-yl- |
| | | | | | | | 1- |
| | | | | | | | methanesulfonyl- |
| | | | | | | | piperidin- |
| | | | | | | | 4-yl)-amide |
| 27 | | | 6.92 uM | 6.92 uM | 32.0 uM | 136% | Propane-2- |
| | | | | | 32.0 uM | 134% | sulfonic acid |
| | | | | | 32.0 uM | 127% | (trans-1- |
| | | | | | | | acetyl-3- |
| | | | | | | | biphenyl-4-yl- |
| | | | | | | | piperidin-4- |
| | | | | | | | yl)-amide |
| 28 | | | 6.44 uM | 6.44 uM | 32.0 uM | 128% | Propane-2- |
| | | | | | 32.0 uM | 132% | sulfonic acid |
| | | | | | 32.0 uM | 116% | [trans-1- |
| | | | | | | | acetyl-3-(4′- |
| | | | | | | | cyano- |
| | | | | | | | biphenyl-4- |
| | | | | | | | yl)-piperidin- |
| | | | | | | | 4-yl]-amide |
| 29 | | | 2.07 uM | 1.73 uM | 31.6 uM | 201% | Propane-2- |
| | | | (1.65-2.60 | 1.44 uM | 31.6 uM | 204% | sulfonic acid |
| | | | n = 10) | 2.16 uM | 31.6 uM | 155% | [trans-1- |
| | | | | 1.96 uM | 31.6 uM | 159% | acetyl-3-(2′- |
| | | | | 1.28 uM | 31.6 uM | 194% | cyano- |
| | | | | 1.51 uM | 31.6 uM | 364% | biphenyl-4- |
| | | | | 2.74 uM | 31.6 uM | 391% | yl)-piperidin- |
| | | | | 2.29 uM | 31.6 uM | 363% | 4-yl]-amide |
| | | | | 3.61 uM | 31.6 uM | 195% | |
| | | | | 3.13 uM | 31.6 uM | 195% | |
| | | | | | 31.6 uM | 187% | |
| | | | | | 31.6 uM | 150% | |
| | | | | | 31.6 uM | 151% | |
| | | | | | 31.6 uM | 155% | |
| | | | | | 31.6 uM | 195% | |
| | | | | | 31.6 uM | 202% | |
| 30 | | | >10.3 uM | 5.39 uM | 32.0 uM | 119% | Propane-2- |
| | | | (0.906-116 | >31.6 uM | 32.0 uM | 113% | sulfonic acid |
| | | | n = 3) | 6.36 uM | 32.0 uM | 126% | (trans-4- |
| | | | | | | | biphenyl-4-yl- |
| | | | | | | | tetrahydro- |
| | | | | | | | pyran-3-yl)- |
| | | | | | | | amide |
| 31 | | | >32.0 uM | >32.0 uM | 32.0 uM | 18.3% | Propane-2- |
| | | | | | 32.0 uM | 11.0% | sulfonic acid |
| | | | | | 32.0 uM | 14.5% | (cis-4- |
| | | | | | | | biphenyl-4-yl- |
| | | | | | | | tetrahydro- |
| | | | | | | | pyran-3-yl)- |
| | | | | | | | amide |
| 32 | | | 2.57 uM | 2.57 uM | 31.6 uM | 120% | Propane-2- |
| | | | | | 31.6 uM | 127% | sulfonic acid |
| | | | | | 31.6 uM | 115% | [trans-4-(4′- |
| | | | | | | | fluoro- |
| | | | | | | | biphenyl-4- |
| | | | | | | | yl)- |
| | | | | | | | tetrahydro- |
| | | | | | | | pyran-3-yl]- |
| | | | | | | | amide |
| 33 | | | 1.94 uM | 1.94 uM | 31.6 uM | 122% | Propane-2- |
| | | | | | 31.6 uM | 121% | sulfonic acid |
| | | | | | | | [trans-4-(2′- |
| | | | | | | | ethoxy- |
| | | | | | | | biphenyl-4- |
| | | | | | | | yl)- |
| | | | | | | | tetrahydro- |
| | | | | | | | pyran-3-yl]- |
| | | | | | | | amide |
| 34 | | | 1.25 uM | 1.25 uM | 31.6 uM | 137% | Propane-2- |
| | | | | | | | sulfonic acid |
| | | | | | | | [trans-4-(2′- |
| | | | | | | | cyano- |
| | | | | | | | biphenyl-4- |
| | | | | | | | yl)- |
| | | | | | | | tetrahydro- |
| | | | | | | | pyran-3-yl]- |
| | | | | | | | amide |
| 35 | | | 8.97 uM | 8.97 uM | 31.6 uM | 146% | Propane-2- |
| | | | | | 31.6 uM | 155% | sulfonic acid |
| | | | | | 31.6 uM | 152% | [trans-4-(4′- |
| | | | | | | | cyano- |
| | | | | | | | biphenyl-4- |
| | | | | | | | yl)- |
| | | | | | | | tetrahydro- |
| | | | | | | | pyran-3-yl]- |
| | | | | | | | amide |
| 36 | | | 7.04 uM | 7.04 uM | pending | pending | Propane-2- |
| | | | | | | | sulfonic acid |
| | | | | | | | [trans-4-(2′- |
| | | | | | | | chloro- |
| | | | | | | | biphenyl-4- |
| | | | | | | | yl)- |
| | | | | | | | tetrahydro- |
| | | | | | | | pyran-3-yl]- |
| | | | | | | | amide |
| 37 | | | >10 uM | >10 uM | Pending | pending | Propane-2- |
| | | | | | | | sulfonic acid |
| | | | | | | | [trans-4-(4′- |
| | | | | | | | methanesulfonyl- |
| | | | | | | | biphenyl- |
| | | | | | | | 4-yl)- |
| | | | | | | | tetrahydro- |
| | | | | | | | pyran-3-yl]- |
| | | | | | | | amide |
| 38 | | | >10 uM | >10 uM | Pending | pending | 4′-[trans-3- |
| | | | | | | | (Propane-2- |
| | | | | | | | sulfonylamino)- |
| | | | | | | | tetrahydro- |
| | | | | | | | pyran-4-yl]- |
| | | | | | | | biphenyl-4- |
| | | | | | | | carboxylic |
| | | | | | | | acid |
| | | | | | | | methylamide |
| 39 | | | 20.2 uM | 20.2 uM | 31.6 uM | 145% | N-{4-[trans-1- |
| | | | | | 31.6 uM | 142% | Acetyl-4- |
| | | | | | | | (propane-2- |
| | | | | | | | sulfonylamino)- |
| | | | | | | | piperidin-3- |
| | | | | | | | yl]-phenyl}- |
| | | | | | | | benzamide |
| 40 | | | 11.7 uM | 11.7 uM | 31.6 uM | 152% | N-{4-[trans-1- |
| | | | | | 31.6 uM | 179% | Acetyl-4- |
| | | | | | 31.6 uM | 176% | (propane-2- |
| | | | | | | | sulfonylamino)- |
| | | | | | | | piperidin-3- |
| | | | | | | | yl]-phenyl}- |
| | | | | | | | 3,5-difluoro- |
| | | | | | | | benzamide |
| 41 | | | 3.05 uM | 3.05 uM | 31.6 uM | 58.3% | Propane-2- |
| | | | | | 31.6 uM | 66.6% | sulfonic acid |
| | | | | | | | ((3S,4S)-rel- |
| | | | | | | | 4-biphenyl-4- |
| | | | | | | | yl-4-hydroxy- |
| | | | | | | | tetrahydro- |
| | | | | | | | pyran-3-yl)- |
| | | | | | | | amide |
| 42 | | | 11.9 uM | 11.9 uM | 31.6 uM | 145% | Propane-2- |
| | | | | | 31.6 uM | 142% | sulfonic acid |
| | | | | | 31.6 uM | 152% | ((3R,4S)-rel- |
| | | | | | | | 4-biphenyl-4- |
| | | | | | | | yl-4-hydroxy- |
| | | | | | | | tetrahydro- |
| | | | | | | | pyran-3-yl)- |
| | | | | | | | amide |
| 43 | | | >31.6 uM | >31.6 uM | 31.6 uM | 85.0% | Propane-2- |
| | | | | | 31.6 uM | 92.2% | sulfonic acid |
| | | | | | 31.6 uM | 102% | (trans-1- |
| | | | | | | | acetyl-4- |
| | | | | | | | biphenyl-4-yl- |
| | | | | | | | piperidin-3- |
| | | | | | | | yl)-amide |
| 44 | | | >31.6 uM | >31.6 uM | 31.6 uM | 1.85% | N-{4-[trans-1- |
| | | | | | 31.6 uM | 1.10% | Methyl-4- |
| | | | | | 31.6 uM | 5.81% | (propane-2- |
| | | | | | | | sulfonylamino)- |
| | | | | | | | piperidin-3- |
| | | | | | | | yl]-phenyl}- |
| | | | | | | | benzamide |
| 45 | | | >31.6 uM | >31.6 uM | 31.6 uM | −0.114% | 3,5-Difluoro- |
| | | | | | 31.6 uM | 0.0432% | N-{4-[trans-1- |
| | | | | | 31.6 uM | −0.344% | methyl-4- |
| | | | | | | | (propane-2- |
| | | | | | | | sulfonylamino)- |
| | | | | | | | piperidin-3- |
| | | | | | | | yl]-phenyl}- |
| | | | | | | | benzamide |

O. Biological Protocols

Materials and Methods

Growth and Maintenance of ES Cells

The murine ES cell line used was E14-Sx1-16C, which has a targeted mutation in the Sox1 gene, a neuroectodermal marker, that offers G418 resistance when the Sox1 gene is expressed (Stem Cell Sciences). ES cells were maintained undifferentiated as previously described (Roach). Briefly, ES cells were grown in SCML media that had a base medium of Knockout™ D-MEM (Invitrogen), supplemented with 15% ES qualified Fetal Bovine Serum (FBS) (Invitrogen), 0.2 mM L-Glutamine (Invitrogen), 0.1 mM MEM non-essential amino acids (Invitrogen), 30 μg/ml Gentamicin (Invitrogen), 1000 u/ml ESGRO (Chemicon) and 0.1 mM 2-Mercaptoethanl (Sigma). ES cells were plated on gelatin-coated dishes (BD Biosciences), the media was changed daily and the cells were dissociated with 0.05% Trypsin EDTA (Invitrogen) every other day.

Neural In Vitro Differentiation of ES Cells

Embryoid Body Formation: Prior to embryoid body (EB) formation the ES cells were weaned from FBS onto Knockout Serum Replacement (KSR) (Invitrogen). To form EBs, ES cells were dissociated into a single cell suspension then 3×106 cells were plated in bacteriology dishes (Nunc 4014) and grown as a suspension culture in NeuroEB-I medium that consisted of Knockout™ D-MEM (Invitrogen), supplemented with 10% KSR (Invitrogen), 0.2 mM L-Glutamine (Invitrogen), 0.1 mM MEM non-essential amino acids (Invitrogen), 30 μg/ml Gentamicin (Invitrogen), 1000 u/ml ESGRO (Chemicon), 0.1 mM 2-Mercaptoethanl (Sigma) and 150 ng/ml Transferrin (Invitrogen). The plates were put on a Stovall Belly Button shaker in an atmospheric oxygen incubator. The media was changed on day 2 of EB formation with NeuroEB-I and on day 4 with NeuroEB-II (NeuroEB-I plus 1 μg/ml mNoggin [R&D Systems]).

Neuronal Precursor Selection and Expansion: On day 5 of EB formation, EBs were dissociated with 0.05% Trypsin EDTA, and 4×10⁶ cells/100 mm dish were plated on Laminin coated tissue culture dishes in NeuroII-G418 medium that consisted of a base medium of a 1:1 mixture of D-MEM/F12 supplemented with N2 supplements and NeuroBasal Medium supplemented with B27 supplement and 0.1 mM L-Glutamine (all from Invitrogen). The base medium was then supplemented with 10 ng/ml bFGF (Invitrogen), 1 μg/ml mNoggin, 500 ng/ml SHH-N, 100 ng/ml FGF-8b (R&D Systems), 1 μg/ml Laminin and 200 μg/ml G418 (Invitrogen) for selection of neuronal precursors expressing Sox-1. The plates were put in an incubator that contained 2% Oxygen and were maintained in these conditions. During the 6-day selection period, the NeuroII media was changed daily. On day 6, the surviving neuronal precursor foci were dissociated with 0.05% Trypsin EDTA and the cells were plated at a density of 1.5×10⁶ cells/100 mm Laminin coated dish in NeuroII-G418 medium. The cells were dissociated every other day for expansion, and prepared for Cryopreservation at passage 3 or 4. The cryopreservation medium contained 50% KSR, 10% Dimethyl Sulfoxide (DMSO) (Sigma) and 40% NeuroI-G418I medium. Neuronas precursors were crypreserved at a concentration of 4×10⁶cells/ml and 1 ml/cryovial in a controlled rate freezer overnight then transferred to an ultra-low freezer or liquid nitrogen for long-term storage,

Neuronal Differentiation: Cryopreserved ES cell-derived neuronal precursors were thawed by the rapid thaw method in a 37-degree water-bath. The cells were transferred from the cryovial to a 100 mm Laminin coated tissue culture dish that already contained NeuroII-G418 that had been equilibrated in a 2% Oxygen incubator. The media was changed with fresh NeuroII-G418 the next day. The cells were dissociated every other day as described above for expansion to generate enough cells to plate for the screen. For the screen, the cells were plated into 384-well poly-d-lysine coated tissue culture dishes (BD Biosciences) by the automated SelecT at a cell density of 6K cells/well in differentiation medium NeuroIII that contained a 4:1 ratio of the NeuroBasalMedium/B27:D-MEM/F12/N2 supplemented with 1 μM cAMP (Sigma), 200 μM Ascorbic Acid (Sigma), 1 μg/ml Laminin (Invitrogen) and 10 ng/ml BDNF (R&D Systems). The plates were put in an incubator with 2% Oxygen and allowed to complete the differentiation process for 7 days. The cells could then be used over a 5-day period for the high throughput screen.

In Vitro Assays

Procedure for AMPA ES Cell FLIPR Screen

FLIPR Methods and Data Analysis:

On the day of the assay, the FLIPR assay is performed using the following methods:

Assay buffer:

| Compound | g/L | MW | [concentration] |
|---|---|---|---|
| NaCl | 8.47 | 58.44 | 145 mM |
| Glucose | 1.8 | 180.2 | 10 mM |
| KCl | .37 | 74.56 | 5 mM |
| MgSO₄ | 1 ml 1M Stock | 246.48 | 1 mM |
| HEPES | 2.38 | 238.3 | 10 mM |
| CaCl₂ | 2 ml 1M Stock | 110.99 | 2 mM |

The pH is adjusted to 7.4 with 1M NaOH. Prepare a 2 mM (approx.) stock solution of Fluo-4, am (Invitrogen) dye in DMSO −22 μl DMSO per 50 μg vial (440 μL per 1 mg vial). Make a 1 mM (approx.) flou-4, PA working solution per vial by adding 22 μl of 20% pluronic acid (PA) (Invitrogen) in DMSO to each 50 μg vial (440 μL per 1 mg vial). Prepare a 250 mM Probenecid (Sigma) stock solution. Make 4 μM (approx.) dye incubation media by adding the contents of 2 50 μg vials per 11 ml DMEM high glucose without glutamine (220 ml DMEM per 1 mg vial). Add 110 μL probenecid stock per 11 ml media (2.5 mM final concentration). Dye concentrations ranging from 2 μM to 8 μM dye can be used without altering agonist or potentiator pharmacology. Add probenecid to the assay buffer used for cell washing (but not drug preparation) at 110 μl probenecid stock per 11 ml buffer.

Remove growth media from cell plates by flicking. Add 50 μl/well dye solution. Incubate 1 hour at 37° C. and 5% CO₂. Remove dye solution and wash 3 times with assay buffer+probenecid (100 μl probenecid stock per 10 ml buffer), leaving 30 μL/well assay buffer. Wait at least 10-15 minutes. Compound and agonist challenge additions are performed with the FLIPR (Molecular Devices). The 1^{st} addition is for test compounds, which are added as 15 μL of a 4× concentration. The second 2^{nd} addition is 15 μL of 4× concentration of agonist or challenge. This achieves 1× concentration of all compounds only after 2^{nd} addition. Compounds are pretreated at least 5 minutes before agonist addition.

Several baseline images are collected with the FLIPR before compound addition, and images are collected for least one minute after compound addition. Results are analyzed by subtracting the minimum fluorescent FLIPR value after compound or agonist addition from the peak fluorescent value of the FLIPR response after agonist addition to obtain the change in fluorescence. The change in fluorescence (RFUs, relative fluorescent units) are then analyzed using standard curve fitting algorithms. The negative control is defined by the AMPA challenge alone, and the positive control is defined by the AMPA challenge plus a maximal concentration of cyclothiazide (10 uM or 32 uM).

Compounds are delivered as DMSO stocks or as powders. Powders are solubilized in DMSO. Compounds are then added to assay drug buffer as 40 μL top [concentration] (4× the top screening concentration). The standard agonist challenge for this assay is 32 uM AMPA.

EC₅₀ values of the compounds of the invention are preferably 10 micromolar or less, more preferably 1 micromolar or less, even more preferably 100 nanomolar or less.

When introducing elements of the present invention or the exemplary embodiment(s) thereof, the articles “a,” “an,” “the” and “said” are intended to mean that there are one or more of the elements. The terms “comprising,”0 “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

1. A compound of formula I, or a pharmaceutically acceptable salt thereof, 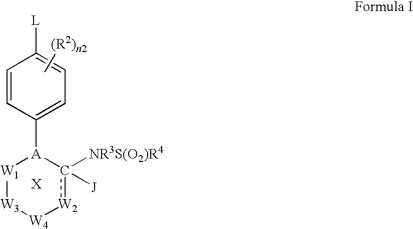
wherein
-L is
a) —Br, —I, —Cl, —O—S(O₂)-alkyl, wherein the —O—S(O₂)-alkyl is optionally substituted with halogen,
b) 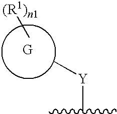
or
c) 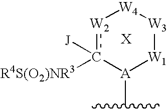
wherein ring G is aryl, heteroaryl, cycloalkyl, or heterocycloalkyl;
each of groups W₁, W₃, and W₄ in each ring X is independently selected from the group consisting of —(CHR¹²)ₐ—, —S(O)₂—, —C(O)—, —O—, —S—, and —NR⁵—;
group W₂ in each ring X is selected from the group consisting of —(CHR¹²)ₐ—, —S(O)₂—, —C(O)—, —O—, —S—, —NR⁵— and N;
J is hydrogen or is absent;
the bond [symbol] between W₂ and C is a single or double bond;
a is independently at each occurrence 1 or 2, provided that if W₃ or W₄ is —(CHR¹²)ₐ—, a is 1;
with the proviso that
(a) a ring X does not contain more than one group selected from —S(O)₂— and —C(O)—;
(b) a ring X contains between one and two ring heteroatoms selected from nitrogen, sulfur or oxygen, wherein if a ring X contains two heteroatoms, then either (i) the two ring heteroatoms are each bonded to a —C(O)— group, or (ii) the two ring heteroatoms are a nitrogen of an —NR⁵— group and a sulfur of an —S(O₂)— group, and the nitrogen and sulfur are directly bonded to each other;
(c) if W₂═N, J is absent and [symbol] is a double bond; and
(d) if both rings X are present, then W₁, W₂, W₃ and W₄ of one ring X are the same as, respectively, W₁, W₂, W₃ and W₄ of the other ring X;
Y is absent or is —O—, —(CR²¹R²²—)ₙ₃, —CR²¹R²²O—, —NR²¹C(O)—, —NR²¹S(O₂), —NR²¹C(O)NR²²—, S(O), or S(O₂);
n3 is 1 or 2;
R²¹ and R²² are each independently hydrogen, alkyl or aryl;
A is C—B, where B is hydrogen, alkyl, halogen, hydroxyl, alkoxy, amino, alkylamino, or dialkylamino;
with the proviso that if W₁ is —O— or —NR⁵—, B is hydrogen, alkyl, hydroxyl or alkoxy;
R³ is hydrogen, alkyl, cycloalkyl, or heterocycloalkyl, wherein each R³ alkyl, cycloalkyl, or heterocycloalkyl is optionally substituted with halogen, —CN, alkoxy, hydroxyl, alkyl, cycloalkyl, heterocycloalkyl, aryl or heteroaryl;
R⁴ is alkyl, alkenyl, cycloalkyl, cycloalkenyl, heterocycloalkyl, aryl, heteroaryl, or NR⁵⁵R⁶⁶, wherein each R⁴ is optionally substituted with halogen, —CN, alkoxy, hydroxyl, alkyl, cycloalkyl, heterocycloalkyl, aryl, heteroaryl;
each of R⁵⁵ and R⁶⁶ is independently hydrogen, alkyl or cycloalkyl, wherein the alkyl or cycloalkyl R⁵⁵ or R⁶⁶ is optionally independently substituted with —R¹⁰¹, —OR¹⁰¹, —C(O)R¹⁰³, or S(O₂)R¹⁰³;
or R⁵⁵ and R⁶⁶ together with the nitrogen they are attached to form a heterocyclic ring which is optionally substituted with one or more alkyl, halogen, or —OR¹⁰¹,
each R⁵ is independently at each occurrence hydrogen alkyl, —C(O)R⁷, —C(O)OR⁷, —C(O)NR⁷R⁸, —S(O₂)R⁷, cycloalkyl, heterocycloalkyl, aryl, or heteroaryl, wherein each R⁵ alkyl, cycloalkyl, heterocycloalkyl, aryl, or heteroaryl is optionally substituted with halogen, —CN, alkoxy, hydroxyl, alkyl, cycloalkyl, heterocycloalkyl, aryl or heteroaryl,
with the proviso that when R⁵ is —C(O)OR⁷ or —C(O)NR⁷R⁸, at least one group bound to the nitrogen of NR⁵ is (CHR¹²);
each of R⁸ and R⁷ is alkyl, cycloalkyl, heterocycloalkyl, wherein each of R⁸ and R⁷ is optionally substituted with halogen, —CN, alkoxy, hydroxyl, alkyl, cycloalkyl, heterocycloalkyl, aryl or heteroaryl;
n1 and n2 are each independently 1, 2, 3 or 4;
each of R¹, R² and R¹² is independently at each occurrence hydrogen, halogen, hydroxyl, alkoxy, cyano, nitro, amino, alkylamino, dialkylamino, C(O)NH₂, C(O)NH(alkyl), C(O)N(alkyl)₂, OC(O)alkyl, C(O)Oalkyl, alkyl, aryl, heteroaryl, heterocycloalkyl, cycloalkyl, or alkyl-S(O)₂—NH—, wherein the R¹, R² and R¹² alkoxy, alkylamino, dialkylamino, C(O)NH(alkyl), C(O)N(alkyl)₂, C(O)Oalkyl, alkyl, aryl, heteroaryl, heterocycloalkyl, cycloalkyl or alkyl-S(O)₂—NH— are each independently optionally substituted with one, two, three or four R⁴¹, wherein each R⁴¹ is independently selected from the group consisting of halogen, —CN, —OR¹⁰¹, alkyl, alkenyl, cycloalkyl, cycloalkenyl, heterocycloalkyl, aryl, heteroaryl, —C(O)R¹⁰¹, —C(O)OR¹⁰¹, —OC(O)OR¹⁰¹, —C(O)NR¹⁰¹R¹⁰², —S(O₂)NR¹⁰¹R¹⁰², —NR¹⁰¹R¹⁰², NR¹⁰¹C(O)R¹⁰³, and —NR¹⁰¹S(O)₂R¹⁰³ wherein each of the R⁴¹ alkyl, heterocycloalkyl, cycloalkyl, aryl or heteroaryl is optionally independently substituted with one or more substituents independently selected from the group consisting of halogen, cyano, —R¹⁰¹, —OR¹⁰¹, —NR¹⁰¹R¹⁰², —S(O)_{q}R¹⁰³, —S(O)₂NR¹⁰¹R¹⁰², —NR¹⁰¹S(O)₂R¹⁰³, —OC(O)R¹⁰³, —C(O)OR¹⁰³, —C(O)NR¹⁰¹R¹⁰², —NR¹⁰¹C(O)R¹⁰³, —NR¹⁰¹C(O)N(R¹⁰³)₂, and —C(O)R¹⁰³;
q is 0, 1 or 2;
or when R¹ is aryl or heteroaryl, two R⁴¹ substituents bonded to adjacent carbon atoms of R¹, together with the adjacent carbon atoms, form a heterocyclic or carbocyclic ring which is optionally substituted with one or more R¹⁰, wherein each R¹⁰ is independently selected from the group consisting of hydrogen, —CN, halogen, —C(O)R¹⁰¹, —C(O)NR¹⁰¹R¹⁰², NR¹⁰¹R¹⁰², —OR¹⁰¹ or —R¹⁰¹;
or, two R¹ substituents bonded to adjacent carbon atoms of ring G, together with the adjacent carbon atoms, form a heterocyclic or carbocyclic ring which is optionally substituted with one or more R¹⁰,
or, two R² substituents bonded to adjacent carbon atoms of the phenyl ring substituted by R², together with the adjacent carbon atoms, form a heterocyclic or carbocyclic ring which is optionally substituted with one or more R¹⁰;
each R¹⁰¹ and each R¹⁰² is independently selected from the group consisting of hydrogen, alkyl, alkenyl, alkynyl, cycloalkyl, aryl, heterocycloalkyl and heteroaryl;
wherein each R¹⁰¹ and R¹⁰² alkyl, alkenyl, alkynyl, cycloalkyl, aryl, heterocycloalkyl or heteroaryl is optionally independently substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl;
each R¹⁰³ is independently selected from the group consisting of alkyl, alkenyl, cycloalkyl, aryl, heterocycloalkyl and heteroaryl and is optionally substituted with one or more substituents independently selected from the group consisting of halogen, hydroxy, cyano, nitro, amino, alkylamino, dialkylamino, alkyl optionally substituted with one or more halogen or alkoxy or aryloxy, aryl optionally substituted with one or more halogen or alkoxy or alkyl or trihaloalkyl, heterocycloalkyl optionally substituted with aryl or heteroaryl or ═O or alkyl optionally substituted with hydroxy, cycloalkyl optionally substituted with hydroxy, heteroaryl optionally substituted with one or more halogen or alkoxy or alkyl or trihaloalkyl, haloalkyl, hydroxyalkyl, carboxy, alkoxy, aryloxy, alkoxycarbonyl, aminocarbonyl, alkylaminocarbonyl and dialkylaminocarbonyl.

2. The compound of claim 1, or a pharmaceutically acceptable salt thereof, wherein Y is absent or is —NHC(O)—.

3. The compound of claim 1, or a pharmaceutically acceptable salt thereof, wherein the compound has the formula I′: 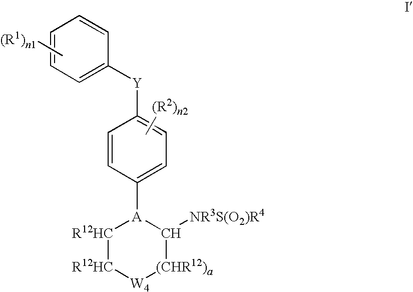
wherein W₄ is —NR⁵— or —O— and a is 1 or 2.

4. The compound of claim 1, or a pharmaceutically acceptable salt thereof, wherein the compound has the formula I″; 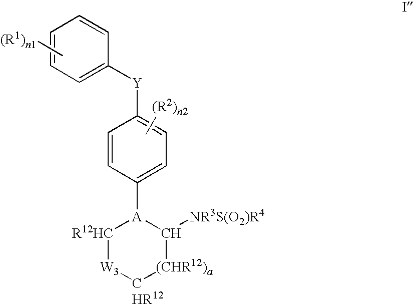
wherein W₃ is —NR— or —O— and a is 1 or 2.

5. The compound of claim 1, or a pharmaceutically acceptable salt thereof, wherein ring G is heterocycloalkyl optionally substituted as in claim 1.

6. The compound of claim 1, or a pharmaceutically acceptable salt thereof, wherein W₃ is —O—.

7. The compound of claim 1, or a pharmaceutically acceptable salt thereof, wherein W₄═—O—.

8. The compound of claim 1, or a pharmaceutically acceptable salt thereof, wherein R⁴ is methyl, ethyl, n-propyl, isopropyl, n-butyl, sec-butyl, isobutyl or t-butyl and R⁴ is optionally substituted with halogen.

9. The compound of claim 1, or a pharmaceutically acceptable salt thereof, wherein ring G is phenyl having one or two R¹ substituents, where each R¹ is independently heteroaryl, cyano, halogen, alkyl, cycloalkyl, alkyl-NH—C(O)—, alkyl-S(O)₂—NH—, halophenyl, or dihalophenyl.

10. The compound of claim 1, or a pharmaceutically acceptable salt thereof, wherein each R¹ is independently hydrogen, phenyl, thiophenyl, halogen, alkoxy, hydroxyl, cyano, C(O)NH₂, C(O)NH(alkyl), C(O)N(alkyl)₂, OC(O)alkyl, C(O)Oalkyl, alkyl, heterocycloalkyl, or cycloalkyl and is independently optionally substituted with one, two, three or four R⁴¹, wherein each R⁴¹ is independently selected from the group consisting of halogen, —C(O)OR¹⁰¹, —OC(O)OR¹⁰¹, —S(O₂)NR¹⁰¹R¹⁰², and —NR¹⁰¹S(O)₂R¹⁰³.

11. The compound of claim 1, or a pharmaceutically acceptable salt thereof, wherein ring G is phenyl and R¹ is in the para position relative to Y.

12. The compound of claim 1, or a pharmaceutically acceptable salt thereof, wherein ring G is phenyl and R¹ is in the ortho position relative to Y.

13. The compound of claim 12, or a pharmaceutically acceptable salt thereof, wherein R¹ is cyano or halogen and is in the ortho or para position relative to Y.

14. A method for the treatment or prevention in a mammal of a condition selected from the group consisting of acute neurological and psychiatric disorders, stroke, cerebral ischemia, spinal cord trauma, head trauma, perinatal hypoxia, cardiac arrest, hypoglycemic neuronal damage, dementia, Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine, urinary incontinence, substance tolerance, substance withdrawal, psychosis, schizophrenia, anxiety, mood disorders, trigeminal neuralgia, hearing loss, tinnitus, macular degeneration of the eye, emesis, brain edema, pain, tardive dyskinesia, sleep disorders, attention deficit/hyperactivity disorder, attention deficit disorder, and conduct disorder, comprising administering a compound of claim 1, or a pharmaceutically acceptable salt thereof, to the mammal.

15. A pharmaceutical composition comprising a compound of claim 1, or a pharmaceutically acceptable salt thereof, and a pharmaceutically acceptable carrier.
